(19) United States
(12) Patent Application Publication
(10) Pub. No.: US 2010/0256143 A1
(43) Pub. Date: Oct. 7, 2010

(54) PHARMACEUTICAL COMPOUNDS

(76) Inventors: Stewart James Baker, Berkshire (GB); Paul John Goldsmith, Berkshire (GB); Timothy Colin Hancox, Berkshire (GB); Neil Anthony Pegg, Berkshire (GB); Stephen Joseph Shuttleworth, Berkshire (GB); Elsa Amandine Dechaux, Essex (GB); Sussie Lerche Krintel, Essex (GB); Stephen Price, Essex (GB); Jonathan Martin Large, Surrey (GB); Edward McDonald, Surrey (GB)

Correspondence Address: VIKSNINS HARRIS & PADYS PLLP, P.O. BOX 111098, ST. PAUL, MN, 55111-1098, US

(21) Appl. No.: 12/594,543

(22) PCT Filed: Apr. 14, 2008
(86) PCT No.: PCT/GB08/01292

§ 371 Date: Apr. 28, 2010

(30) Foreign Application Priority Data
Apr. 12, 2007 (GB) 0707086.5
Apr. 19, 2007 (GB) 0707613.6

(51) Int. Cl. A61K 31/5377; C07D 413/14; A61P 35/00; A61P 31/12; A61P 37/00; A61P 29/00; A61P 25/00; A61P 9/00; A61P 3/00

(52) U.S. Cl. 514/235.2; 544/122

(57) ABSTRACT

The invention provides a pyrimidine compound of formula (I):

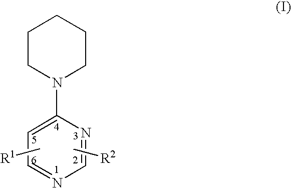

wherein R¹ and R² have any of the values defined herein; or a pharmaceutically acceptable salt thereof. The compounds are inhibitors of PI3K and may thus be used to treat diseases and disorders arising from abnormal cell growth, function or behaviour associated with PI3 kinase such as cancer, immune disorders, cardiovascular disease, viral infection, inflammation, metabolism/endocrine function disorders and neurological disorders.

### FIELD OF THE INVENTION

The present invention relates to pyrimidine compounds and to their use as inhibitors of phosphatidylinositol 3-kinase (PI3K).

### BACKGROUND TO THE INVENTION

Phosphatidylinositol (hereinafter abbreviated as “PI”) is one of a number of phospholipids found in cell membranes. In recent years it has become clear that PI plays an important role in intracellular signal transduction. In the late 1980s, a PI3 kinase (PI3K) was found to be an enzyme which phosphorylates the 3-position of the inositol ring of phosphatidylinositol (D. Whitman et al, 1988, Nature, 332, 664).

PI3K was originally considered to be a single enzyme, but it has now been clarified that a plurality of subtypes are present in PI3K. Each subtype has its own mechanism for regulating activity. Three major classes of PI3Ks have been identified on the basis of their in vitro substrate specificity (B. Vanhaesebroeck, 1997, Trend in Biol. Sci, 22, 267). Substrates for class I PI3Ks are PI, PI 4-phosphate (PI4P) and PI 4,5-biphosphate (PI(4,5)P2). Class I PI3Ks are further divided into two groups, class Ia and class Ib, in terms of their activation mechanism. Class Ia PI3Ks include PI3K p110α, p110β and p110δ subtypes, which transmit signals from tyrosine kinase-coupled receptors. Class Ib PI3K includes a p110γ subtype activated by a G protein-coupled receptor. PI and PI(4)P are known as substrates for class II PI3Ks. Class II PI3Ks include PI3K C2α, C2β and C2γ subtypes, which are characterized by containing C2 domains at the C terminus. The substrate for class III PI3Ks is PI only.

In the PI3K subtypes, the class Ia subtype has been most extensively investigated to date. The three subtypes of class Ia are heterodimers of a catalytic 110 kDa subunit and regulatory subunits of 85 kDa or 55 kDa. The regulatory subunits contain SH2 domains and bind to tyrosine residues phosphorylated by growth factor receptors with a tyrosine kinase activity or oncogene products, thereby inducing the PI3K activity of the p110 catalytic subunit which phosphorylates its lipid substrate. Thus, the class Ia subtypes are considered to be associated with cell proliferation and carcinogenesis, immune disorders and conditions involving inflammation.

WO 01/083456 describes a series of condensed heteroaryl derivatives which have activity as inhibitors of PI3 K and which suppress cancer cell growth.

### SUMMARY OF THE INVENTION

It has now been found that a series of novel pyrimidine compounds have activity as inhibitors of PI3K. The compounds exhibit selectivity for class Ia PI3Ks over class Ib, in particular for the p110δ subtype. Accordingly, the present invention provides a compound which is a pyrimidine of formula (I):

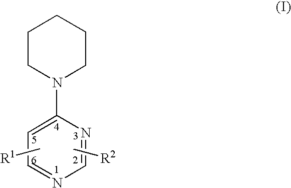

wherein

R² is bonded at ring position 2 and R¹ is bonded at ring position 5 or 6, or R¹ is bonded at ring position 2 and R² is bonded at ring position 6;

R¹ is selected from —(CR₂)ₙ—Y—R³, -[arylene-(CR₂)ₙ]ₚNR⁴R⁵, -[heteroarylene-(CR₂)ₙ]ₚ—NR⁴R⁵, —C(O)NR¹⁰R¹¹ and —O—(CR′R″)ₙ—R³;

R² is an indole group which is unsubstituted or substituted;

Y is selected from a direct bond, —O—(CR₂)ₙ—, —O—(CR₂)ₙ—NR—, —NR—(CR₂)ₙ—, —NR—(CR₂)ₙO—(CR₂)ₙ—, —NR—(CR₂)ₙ—C(O)—, —(CR₂)—(CR₂)ₙ—, —S(O)_{q}(CR₂)ₙ—, —N(SO₂R)—(CR₂)ₙ—, NRC(O)—(CR₂)ₙ, —C(O)NR—(CR₂)ₙ—, —NRSO₂—(CR₂)ₙ, and —SO₂NR—(CR₂)ₙ;

m is 1, 2 or 3;

n is 0, 1, 2 or 3;

p is 0 or 1;

q is 0, 1 or 2;

each R, which are the same or different when more than one is present in a given group, is independently H or C₁-C₆ alkyl which is unsubstituted or substituted;

one of R′ and R″ is H and the other is C₁-C₆ alkyl which is unsubstituted or substituted, or each of R′ and R″, which are the same or different, is C₁-C₆ alkyl which is unsubstituted or substituted;

R³ is selected from an unsaturated 5- to 12-membered carbocyclic or heterocyclic ring, a saturated 5-, 6- or 7-membered N-containing heterocyclic group which is unsubstituted or substituted, a group —OR and a group —NR⁶R⁷;

one of R⁴ and R⁵ is H and the other is a saturated 5-, 6- or 7-membered N-containing heterocyclic group which is unsubstituted or substituted, or one of R⁴ and R⁵ is unsubstituted C₁-C₆ alkyl and the other is C₁-C₆ alkyl substituted by an unsaturated 5- to 12-membered carbocyclic or heterocyclic ring which is unsubstituted or substituted, or R⁴ and R⁵, which are the same or different, are both C₁-C₆ alkyl substituted by an unsaturated 5- to 12-membered carbocyclic or heterocyclic ring which is unsubstituted or substituted, or R⁴ and R⁵ together form, with the nitrogen atom to which they are attached, a saturated 5-, 6- or 7-membered N-containing heterocyclic group which is unsubstituted or substituted or which is fused to a benzene ring;

R⁶ and R⁷, which are the same or different, are each independently selected from H and C₁-C₆ alkyl which is unsubstituted or substituted, or R⁶ and R⁷ together form, with the nitrogen atom to which they are attached, a saturated 5-, 6- or 7-membered N-containing heterocyclic ring which is unsubstituted or substituted or which is fused to a second saturated 5-, 6- or 7-membered N-containing heterocyclic ring; and

R¹⁰ and R¹¹, which are the same or different, are each C₁-C₆ alkyl which is unsubstituted or substituted, or one of R¹⁰ and R¹¹ is H and the other is a saturated 5-, 6- or 7-membered N-containing heterocyclic group which is unsubstituted or substituted, or one of R¹⁰ and R¹¹ is unsubstituted C₁-C₆ alkyl and the other is C₁-C₆ alkyl substituted by an unsaturated 5- to 12-membered carbocyclic or heterocyclic ring which is unsubstituted or substituted, or R¹⁰ and R¹¹, which are the same or different, are both C₁-C₆ alkyl substituted by an unsaturated 5- to 12-membered carbocyclic or heterocyclic ring which is unsubstituted or substituted, or R¹⁰ and R¹¹ together form, with the nitrogen atom to which they are attached, a saturated 5-, 6- or 7-membered N-containing heterocyclic group which is unsubstituted or substituted or which is fused to a benzene ring;

or a pharmaceutically acceptable salt thereof;

with the proviso that when one of R⁴ and R⁵ is unsubstituted C₁-C₆ alkyl and the other is C₁-C₆ alkyl substituted by an unsaturated 5- to 12-membered carbocyclic or heterocyclic ring which is unsubstituted or substituted, or R⁴ and R⁵, which are the same or different, are both C₁-C₆ alkyl substituted by an unsaturated 5- to 12-membered carbocyclic or heterocyclic ring which is unsubstituted or substituted, then R² is other than an indol-4-yl group which is substituted at the 5- or 6-position.

### DETAILED DESCRIPTION OF THE INVENTION

A C₁-C₆ alkyl group is linear or branched. A C₁-C₆ alkyl group is typically a C₁-C₄ alkyl group, for example a methyl, ethyl, propyl, n-butyl, sec-butyl or tert-butyl group. A C₁-C₆ alkyl group is unsubstituted or substituted, typically by one or more groups Z or R⁹ as defined below. Typically it is C₁-C₄ alkyl, for example methyl, ethyl, i-propyl, n-propyl, t-butyl, s-butyl or n-butyl.

Z is selected from H, unsubstituted C₁-C₆ alkyl, halo, —OR, —SR, CH₂OR, —CF₃, -(halo)-C₁-C₆ alkyl, —(C(R⁸)₂)_{q}O-(halo)-C₁-C₆ alkyl, —CO₂R, —(C(R⁸)₂)_{q}CO₂R, —(C(R⁸)₂)_{q}COR, CF₂OH, CH(CF₃)OH, C(CF₃)₂OH, —(CH₂)_{q}OR, —(C(R⁸)₂)_{q}OR, —(CH₂)_{q}NR₂, —(C(R⁸)₂)_{q}NR₂, —C(O)N(R)₂, —(C(R⁸)₂)_{q}CONR₂, —NR₂, —(C(R⁸)₂)_{q}NR₂, —NRC(O)R, —(C(R⁸)₂)_{q}NRC(O)OR, —S(O)ₘR, —S(O)ₘN(R)₂, —(C(R⁸)₂)_{q}S(O)ₘN(R)₂, —OC(O)R, —(C(R⁸)₂)_{q}OC(O)R, —OC(O)N(R)₂, —(C(R⁸)₂)_{q}OC(O)N(R)₂, —(C(R⁸)₂)_{q}OC(O)NR₂, —NRS(O)ₘR, —(C(R⁸)₂)_{q}NRS(O)ₘR, —NRC(O)N(R)₂, —(C(R⁸)₂)_{q}NRC(O)N(R)₂, CN, —NO₂ and a 5- to 12-membered aryl or heteroaryl group, which group is unsubstituted or substituted, wherein each R is independently selected from H, C₁-C₆ alkyl, C₃-C₁₀ cycloalkyl and a 5- to 12-membered aryl or heteroaryl group, the group being unsubstituted or substituted, m is 1 or 2 and q is 0, 1 or 2.

R⁹ is selected from C₁-C₆ alkoxy, OR⁸, SR⁸, S(O)ₘR⁸, nitro, CN, halogen, —C(O)R⁸, —CO₂R⁸, —C(O)N(R⁸)₂ and —N(R⁸)₂.

R⁸, each of which is the same or different when more than one is present in a given substituent, is selected from H, C₁-C₆ alkyl and C₃-C₁₀ cycloalkyl, and m is 1 or 2.

A halogen or halo group is F, Cl, Br or I. Preferably it is F, Cl or Br. A C₁-C₆ alkyl group substituted by halogen may be denoted by the term “halo-C₁-C₆ alkyl”, which means an alkyl group in which one or more hydrogens is replaced by halo. A halo-C₁-C₆ alkyl group preferably contains one, two or three halo groups. A preferred example of such a group is trifluoromethyl.

A C₁-C₆ alkoxy group is linear or branched. It is typically a C₁-C₄ alkoxy group, for example a methoxy, ethoxy, propoxy, i-propoxy, n-propoxy, n-butoxy, sec-butoxy or tert-butoxy group. A C₁-C₆ alkoxy group is unsubstituted or substituted, typically by one or more groups Z or R⁹ as defined above.

A C₃-C₁₀ cycloalkyl group may be, for instance, C₃-C₈ cycloalkyl such as cyclopropyl, cyclobutyl, cyclopentyl, cyclohexyl, or cycloheptyl. Typically it is C₃-C₆ cycloalkyl. A C₃-C₁₀ cycloalkyl group is unsubstituted or substituted, typically by one or more groups Z or R⁹ as defined above.

In an alkylene chain —(CR₂)ₘ— or —(CR₂)ₙ—, the units CR₂ may be the same or different when m or n is greater than 1.

An arylene or heteroarylene group is a divalent aryl or heteroaryl group as defined herein.

A saturated 5-, 6-, or 7-membered N-containing heterocyclic group typically contains one nitrogen atom and either an additional N atom or an O or S atom, or no additional heteroatoms. It may be, for example, piperidine, piperazine, morpholine, thiomorpholine, pyrrolidine or homopiperazine. Examples of a 5-, 6- or 7-membered N-containing saturated heterocyclic group which is fused to a second saturated 5-, 6- or 7-membered N-containing saturated heterocyclic group include octahydro-pyrrolo[1,2-a]pyrazine, octahydro-pyrrolo[3,4-c]pyrrole 3,9-diazaspiro[5.5]undecane, 2,7-diazaspiro[3.5]nonane, 2,8-diazaspiro[4.5]decane and 2,7-diazaspiro[4.4]nonane.

The saturated 5-, 6-, or 7-membered N-containing heterocyclic group is unsubstituted or substituted on one or more ring carbon atoms and/or on any additional N atom present in the ring. Examples of suitable substituents include one or more groups Z or R⁹ as defined above, and a C₁-C₆ alkyl group which is unsubstituted or substituted by a group Z or R⁹ as defined above. When the ring is piperazine it is typically unsubstituted or substituted, typically on the second ring nitrogen atom, by —C(O)R⁸, —C(O)N(R⁸)₂ or —S(O)ₘR⁸, or by C₁-C₆ alkyl which is unsubstituted or substituted by C₁-C₆ alkoxy or OH.

An unsaturated 5- to 12-membered carbocyclic group is a 5-, 6-, 7-, 8-, 9-, 10, 11- or 12-membered carbocyclic ring containing at least one unsaturated bond. It is a monocyclic or fused bicyclic ring system. The group is aromatic or non-aromatic, for instance a 5- to 12-membered aryl group. Examples include phenyl, naphthyl, indanyl, indenyl and tetrahydronaphthyl groups. The group is unsubstituted or substituted, typically by one or more groups Z or R⁹ as defined above.

An aryl group is a 5- to 12-membered aromatic carbocyclic group. It is monocyclic or bicyclic. Examples include phenyl and naphthyl groups. The group is unsubstituted or substituted, for instance by a group Z or R⁹ as defined above.

An unsaturated 5- to 12-membered heterocyclic group is a 5-, 6-, 7-, 8-, 9-, 10, 11- or 12-membered heterocyclic ring containing at least one unsaturated bond and at least one heteroatom selected from O, N and S. It is a monocyclic or fused bicyclic ring system. The group is aromatic or non-aromatic, for instance heteroaryl. The group may be, for example, furan, thiophene, pyrrole, pyrrolopyrazine, pyrrolopyrimidine, pyrrolopyridine, pyrrolopyridazine, indole, isoindole, pyrazole, pyrazolopyrazine, pyrazolopyrimidine, pyrazolopyridine, pyrazolopyridazine, imidazole, imidazopyrazine, imidazopyrimidine, imidazopyridine, imidazopyridazine, benzimidazole, benzodioxole, benzodioxine, benzoxazole, benzothiophene, benzothiazole, benzofuran, indole, indolizinyl, isoxazole, oxazole, oxadiazole, thiazole, isothiazole, thiadiazole, dihydroimidazole, dihydrobenzofuran, dihydrodioxinopyridine, dihydropyrrolopyridine, dihydrofuranopyridine, dioxolopyridine, pyridine, quinoline, isoquinoline, quinazoline, quinoxaline, tetrahydrobenzofuran, tetrahydroquinoline, tetrahydroisoquinoline, 5,6,7,8-tetrahydro-imidazo[1,5-a]pyrazine, 5,6,7,8-tetrahydro-imidazo[1,2-a]pyrazine, thienopyrazine, pyrimidine, pyridazine, pyrazine, triazine, triazole or tetrazole. The group is unsubstituted or substituted, typically by one or more groups Z or R⁹ as defined above.

Heteroaryl is a 5- to 12-membered aromatic heterocyclic group which contains 1, 2, 3, or 4 heteroatoms selected from O, N and S. It is monocyclic or bicyclic. Typically it contains one N atom and 0, 1, 2 or 3 additional heteroatoms selected from O, S and N. It may be, for example, selected from the heteroaryl groups in the above list of options for a 5 to 12-membered heterocyclic group.

R¹ is typically a group —(CR₂)ₘ—Y—R³ as defined above.

When R³ is an unsaturated 5- to 12-membered carbocyclic group as defined above it is typically an aromatic carbocyclic group such as phenyl or naphthyl. When R³ is an unsaturated 5- to 12-membered heterocyclic group it is typically pyridyl, for instance a pyrid-2-yl, pyrid-3-yl or pyrid-4-yl group. When R³ is a saturated 5-, 6- or 7-membered N-containing heterocyclic group it is typically a 6-membered such heterocyclic group, for instance piperidyl, morpholinyl or piperazinyl. The group R³ is unsubstituted or substituted, for instance by a group Z or R⁹ as defined above.

R² is an indolyl group which is unsubstituted or substituted. The indolyl group may be linked to the pyrimidine core via any available ring position. It may, for instance, be an indol-4-yl, indol-5-yl, indol-6-yl or indol-7-yl group. Typically it is indol-4-yl or indol-6-yl, more typically an indol-4-yl group.

When substituted, the indolyl may be substituted at one or more available ring positions. Typically it bears a substituent on the benzene moiety of the indole group. For instance, an indol-4-yl group is typically substituted at the 5-, 6- or 7-position, more typically at the 5- or 6-position. An indol-5-yl group is typically substituted at the 4-, 6- or 7-position, more typically at the 4- or 6-position. An indol-6-yl group is typically substituted at the 4-, 5- or 7-position, more typically at the 4- or 5-position. An indol-7-yl group is typically substituted at the 4-, 5- or 6-position, more typically at the 5- or 6-position.

Examples of suitable substituents for the indolyl group include CN, halo, —C(O)NR₂, halo(C₁-C₆)alkyl such as CF₃, —SO₂R, —SO₂NR₂, and a 5-membered heteroaryl group containing 1, 2, 3 or 4 heteroatoms selected from O, N and S, wherein R is H or C₁-C₆ alkyl. Typically the substituent is an electron-withdrawing group.

The 5-membered heteroaryl group may be, for example, furan, thiophene, pyrrole, imidazole, pyrazole, triazole, tetrazole, oxazole, isoxazole, oxadiazole, thiazole, isothiazole, or thiadiazole.

In one embodiment a substituted indolyl group is an indol-4-yl group substituted at the 5- or 6-position, in particular the 6-position, by CN, halo, —C(O)NH₂, —CF₃, —SO₂Me, —SO₂NMe₂ or a 5-membered heteroaryl group as defined above. Typically the indol-4-yl group is substituted at the 5- or 6-position by halo, in particular by F. More typically the indol-4-yl group is substituted at the 6-position by halo, in particular by F.

Y is typically selected from —O(CR₂)ₙ—, —NR—(CR₂)ₙ—, —NR—(CR₂)ₘO—, and —(CR₂)—(CR₂)ₙ—.

A group —O(CR)ₙ— is typically —O—, —OCH₂—, —OCH(Me)-, —OCH₂CH₂—, OCH₂CH(Me)- or —OCH(Me)CH₂—.

A group —NR—(CR₂)ₙ— is typically —NH—, —NMe—, —NHCH₂—, —NHCH(Me)-, —NHCH₂CH₂—, —NHCH₂CH(Me)-, —NHCH(Me)CH₂—, —N(Me)CH₂— or N(Me)CH₂CH₂—. In particular it is —NHCH₂—, —NHCH(Me)-, —NHCH₂CH₂—, —N(Me)CH₂— or —N(Me)CH₂CH₂—.

A group —NR—(CR₂)ₘ—O— is typically —NHCH₂CH₂—O—, —NHCH₂CH(Me)-O—, —NHCH(Me)CH₂—O— or —N(Me)CH₂CH₂—O—. In particular it is —NHCH₂CH₂—O— or —N(Me)CH₂CH₂—O—.

A group —(CR₂)—(CR₂)ₙ— is typically —CH₂—, —CHMe—, —CH₂CH₂—, —CH(Me)CH₂— or —CH₂CH(Me)-.

An arylene or heteroarylene group may, for instance, be selected from:

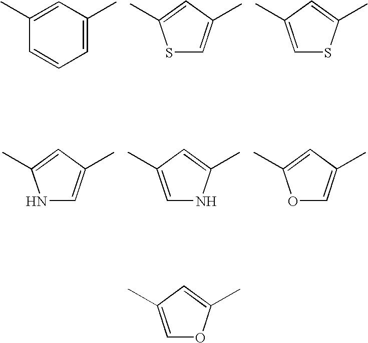

In one embodiment the pyrimidine is of formula (Ia):

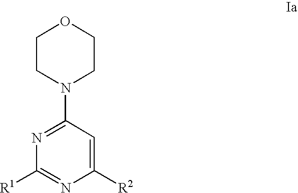

wherein R¹ and R² are as defined above for formula (I).

In formula (Ia) R¹ is typically —NR⁴R⁵ or —(CH₂)ₘ—Y—R³ wherein m is 1 or 2; Y is selected from a direct bond, —NH—CH₂—, —NH—(CH₂)₂—, —N(Me)CH₂—, —NHCH(Me)-, —NHC(O)— and —N(Me)C(O)—; and R³ is as defined for formula (I). Typically R³ is an unsaturated 5- to 12-membered carbocyclic or heterocyclic ring which is unsubstituted or substituted, for instance a phenyl, pyridyl, imidazolyl or tetrahydroisoquinolinyl ring, or R³ is a saturated 5-, 6- or 7-membered heterocyclic ring which is unsubstituted or substituted, for instance a piperidyl, piperazinyl or morpholinyl ring. A pyridyl ring is typically pyrid-2-yl, pyrid-3-yl or pyrid-4-yl. An imidazolyl ring is typically imidazol-2-yl, imidazol-4-yl or imidazol-5-yl. R² is an indole group which is unsubstituted or substituted. Typically R² is an indol-4-yl or indol-6-yl group which is unsubstituted or substituted. When the indole group is substituted it is typically substituted by halo, CN, CF₃, —CONH₂, —SO₂NMe₂ or —SO₂Me, for instance at the 5- or 6-position.

In a second embodiment the pyrimidine is of formula (Ib):

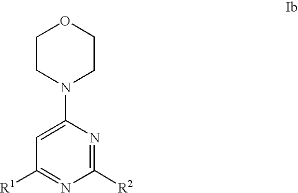

wherein R¹ and R² are as defined above for formula (I).

In formula (Ib), R¹ is typically —NR⁴R⁵ or a group —(CH₂)ₘ—Y—R³ in which m is 1 or 2; Y is selected from a direct bond, —NHCH₂—, —N(Me)CH₂—, —NHCH₂CH₂—, —N(Me)(CH₂)₂—, —NHCH(Me)- and —N(Me)CH₂—; and R³ is as defined above for formula (I). Typically R³ is an unsaturated 5- to 12-membered carbocyclic or heterocyclic ring which is unsubstituted or substituted, for instance a phenyl, pyridyl, imidazolyl or tetrahydroisoquinolinyl ring; or R³ is a saturated 5-, 6- or 7-membered heterocyclic ring which is unsubstituted or, for instance a piperidyl, piperazinyl or morpholinyl ring. A pyridyl ring is typically pyrid-2-yl, pyrid-3-yl or pyrid-4-yl. An imidazolyl ring is typically imidazol-4-yl or imidazol-5-yl. R² is an indole group which is unsubstituted or substituted. When the indole group is substituted it is typically substituted as defined above for formula (Ia).

In a third embodiment the pyrimidine is of formula (Ic):

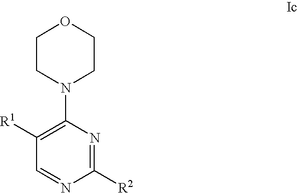

wherein R¹ and R² are as defined above for formula (I).

Specific examples of compounds of the invention include those listed in the following Table 1:

TABLE 1

| Compound | | |
|---|---|---|
| No. | Structure | Name |
| 1 | 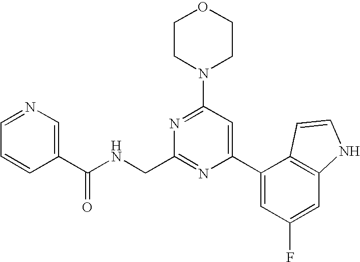 | N-[4-(6-Fluoro-1H-indol-4-yl)-6- morpholin-4-yl-pyrimidin-2-ylmethyl]- nicotinamide |
| 2 | 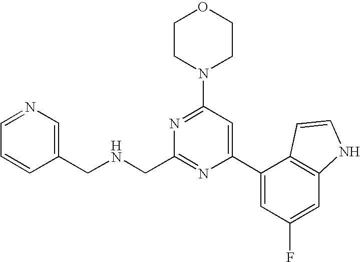 | 4-(6-Fluoro-1H-indol-4-yl)-6-morpholin-4- yl-pyrimidin-2-ylmethyl]-pyridin-3- ylmethyl-amine |
| 3 | 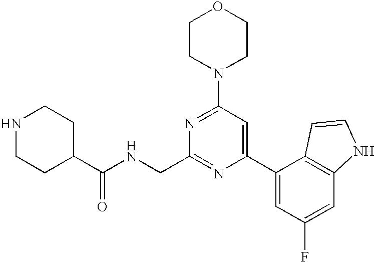 | Piperidine-4-carboxylic acid [4-(6-fluoro- 1H-indol-4-yl)-6-morpholin-4-yl-pyrimidin- 2-ylmethyl]-amide |
| 4 | 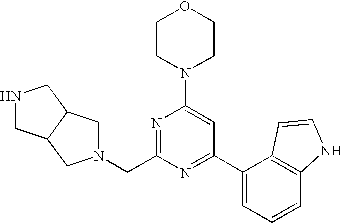 | 4-[2-(Hexahydro-pyrrolo[3,4-c]pyrrol-2- ylmethyl)-6-morpholin-4-yl-pyrimidin-4- yl]-1H-indole |
| 5 | 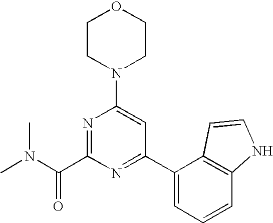 | 4-(1H-indol-4-yl)-6-morpholin-4-yl- pyrimidine-2-carboxylic acid dimethylamide |
| 6 | 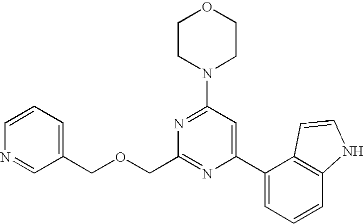 | 4-[6-Morpholin-4-yl-2-(pyridin-3- ylmethoxymethyl)-pyrimidin-4-yl]-1H- indole |
| 7 | 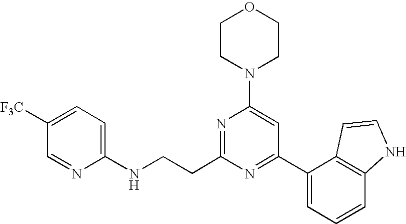 | {2-[4-(1H-Indol-4-yl)-6-morpholin-4-yl- pyrimidin-2-yl]-ethyl}-(5-trifluoromethyl- pyridin-2-yl)-amine |
| 8 | 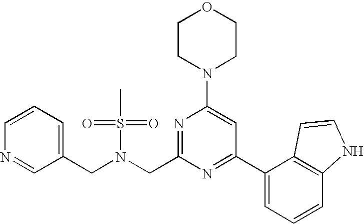 | N-[4-(1H-Indol-4-yl)-6-morpholin-4-yl- pyrimidin-2-ylmethyl]-N-pyridin-3- ylmethyl-methanesulfonamide |
| 9 | 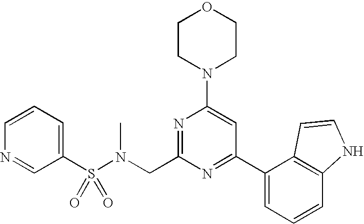 | Pyridine-3-sulfonic acid [4-(1H-indol-4-yl)- 6-morpholin-4-yl-pyrimidin-2-ylmethyl]- methyl-amide |
| 10 | 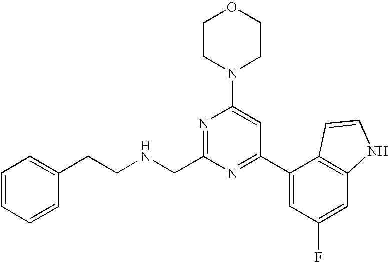 | [4-(6-Fluoro-1H-indol-4-yl)-6-morpholin-4- yl-pyrimidin-2-ylmethyl]-phenethyl-amine |
| 11 | 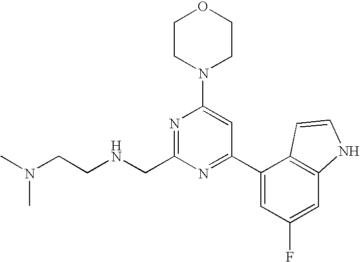 | N′-[4-(6-Fluoro-1H-indol-4-yl)-6- morpholin-4-yl-pyrimidin-2-ylmethyl]-N,N- dimethyl-ethane-1,2-diamine |
| 12 | 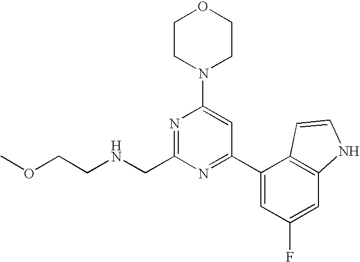 | [4-(6-Fluoro-1H-indol-4-yl)-6-morpholin-4- yl-pyrimidin-2-ylmethyl]-(2-methoxy- ethyl)-amine |
| 13 | 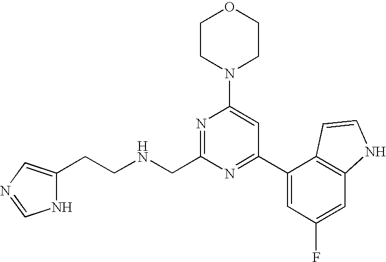 | [4-(6-Fluoro-1H-indol-4-yl)-6-morpholin-4- yl-pyrimidin-2-ylmethyl]-[2-(3H-imidazol- 4-yl)-ethyl]-amine |
| 14 | 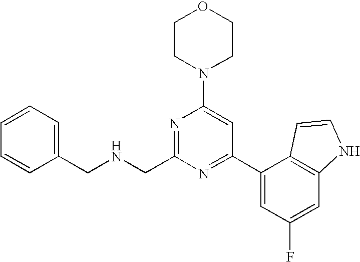 | Benzyl-[4-(6-fluoro-1H-indol-4-yl)-6- morpholin-4-yl-pyrimidin-2-ylmethyl]- amine |
| 15 | 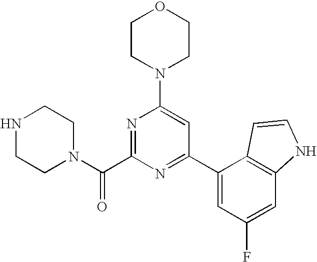 | [4-(6-Fluoro-1H-indol-4-yl)-6-morpholin-4- yl-pyrimidin-2-yl]-piperazin-1-yl- methanone |
| 16 | 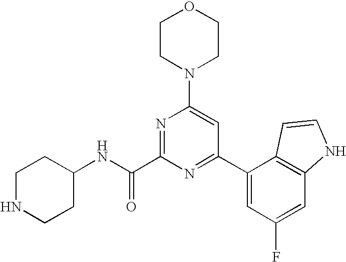 | 4-(6-Fluoro-1H-indol-4-yl)-6-morpholin-4- yl-pyrimidine-2-carboxylic acid piperidin-4- ylamide |
| 17 | 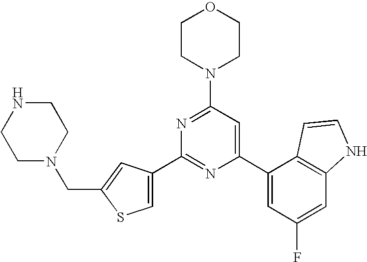 | 6-Fluoro-4-[6-morpholin-4-yl-2-(5- piperazin-1-ylmethyl-thiophen-3-yl)- pyrimidin-4-yl]-1H-indole |
| 18 | 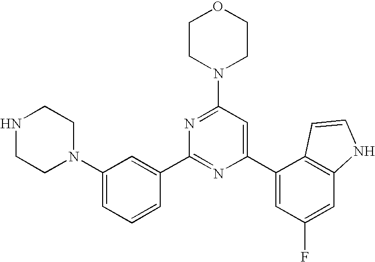 | 6-Fluoro-4-[6-morpholin-4-yl-2-(3- piperazin-1-yl-phenyl)-pyrimidin-4-yl]-1H- indole |
| 19 | 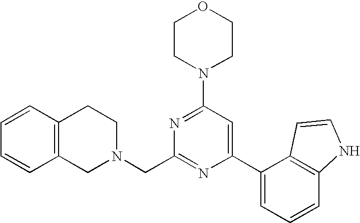 | 2-[4-(1H-Indol-4-yl)-6-morpholin-4-yl- pyrimidin-2-ylmethyl]-1,2,3,4-tetrahydro- isoquinoline |
| 20 | 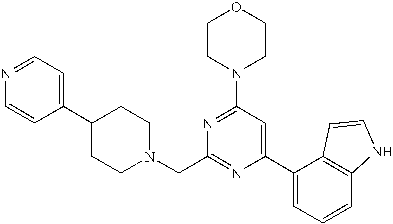 | 1-[4-(1H-Indol-4-yl)-6-morpholin-4-yl- pyrimidin-2-ylmethyl]-1,2,3,4,5,6- hexahydro-[4,4′]bipyridinyl |
| 21 | 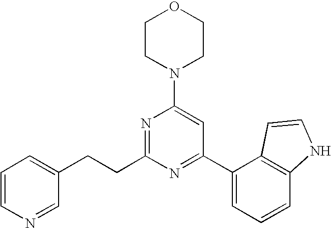 | 4-[6-Morpholin-4-yl-2-(2-pyridin-3- ethyl)-pyrimidin-4-yl]-1H-indole |
| 22 | 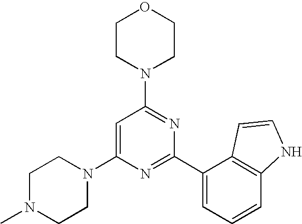 | 4-[4-(4-Methyl-piperazin-1-yl)-6- morpholin-4-yl-pyrimidin-2-yl]-1H-indole |
| 23 | 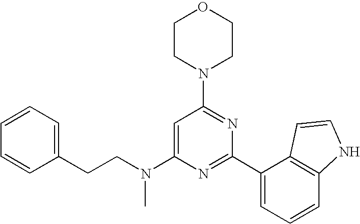 | [2-(1H-Indol-4-yl)-6-morpholin-4-yl- pyrimidin-4-yl]-methyl-phenethyl-amine |
| 24 | 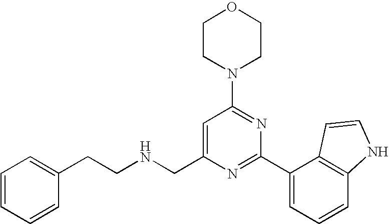 | 2-(1H-Indol-4-yl)-6-morpholin-4-yl- pyrimidin-4-ylmethyl]-phenethyl-amine |
| 25 | 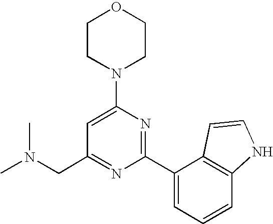 | [2-(1H-Indol-4-yl)-6-morpholin-4-yl- pyrimidin-4-ylmethyl]-dimethyl-amine |
| 26 | 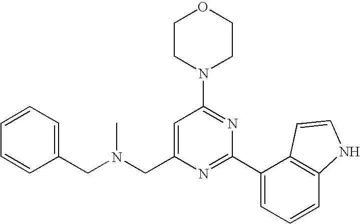 | Benzyl-[2-(1H-indol-4-yl)-6-morpholin-4- yl-pyrimidin-4-ylmethyl]-methyl-amine |
| 27 | 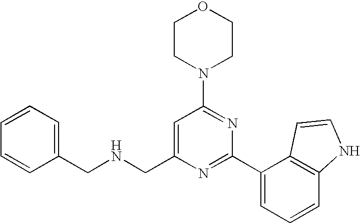 | Benzyl-[2-(1H-indol-4-yl)-6-morpholin-4- yl-pyrimidin-4-ylmethyl]-amine |
| 28 | 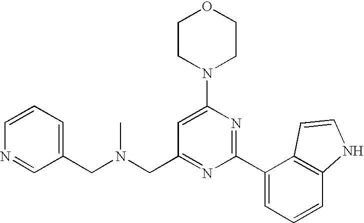 | [2-(1H-Indol-4-yl)-6-morpholin-4-yl- pyrimidin-4-ylmethyl]-methyl-pyridin-3- ylmethyl-amine |
| 29 | 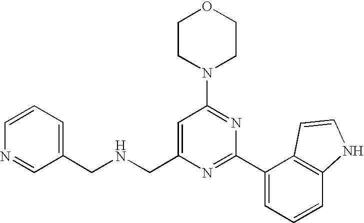 | [2-(1H-Indol-4-yl)-6-morpholin-4-yl- pyrimidin-4-ylmethyl]-pyridin-3-ylmethyl- amine |
| 30 | 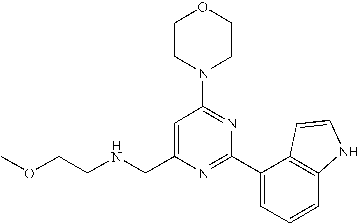 | [2-(1H-Indol-4-yl)-6-morpholin-4-yl- pyrimidin-4-ylmethyl]-(2-methoxy-ethyl)- amine |
| 31 | 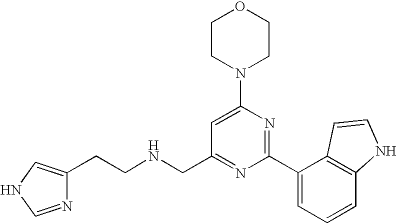 | [2-(1H-imidazol-4-yl)-ethyl]-[2-(1H-indol- 4-yl)-6-morpholin-4-yl-pyrimidin-4- ylmethyl]-amine |
| 32 | 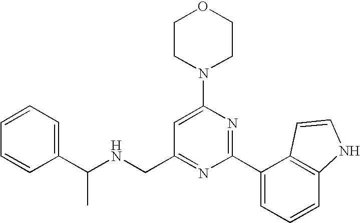 | [2-(1H-Indol-4-yl)-6-morpholin-4-yl- pyrimidin-4-ylmethyl]-(1-phenyl-ethyl)- amine |
| 33 | 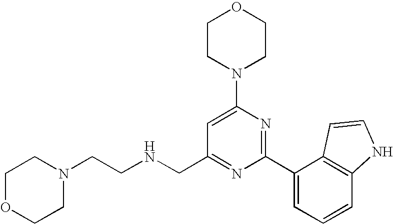 | [2-(1H-Indol-4-yl)-6-morpholin-4-yl- pyrimidin-4-ylmethyl]-(2-morpholin-4-yl- ethyl)-amine |
| 34 | 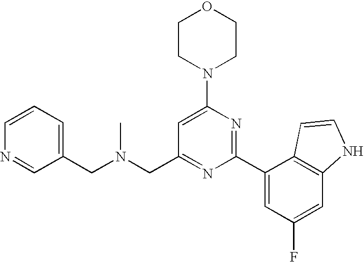 | [2-(6-Fluoro-1H-indol-4-yl)-6-morpholin-4- yl-pyrimidin-4-ylmethyl]-methyl-pyridin-3- ylmethyl-amine |
| 35 | 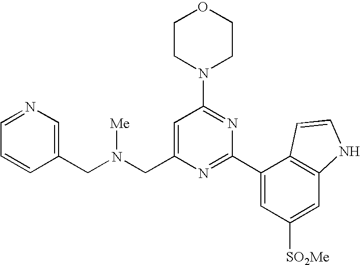 | [2-(6-Methanesulfonyl-1H-indol-4-yl)-6- morpholin-4-yl-pyrimidin-4-ylmethyl]- methyl-pyridin-3-ylmethyl-amine |
| 36 | 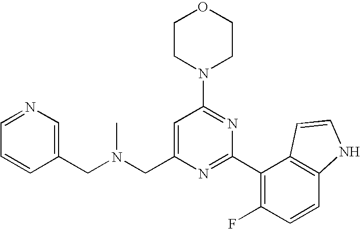 | [2-(5-Fluoro-1H-indol-4-yl)-6-morpholin-4- yl-pyrimidin-4-ylmethyl]-methyl-pyridin-3- ylmethyl-amine |
| 37 | 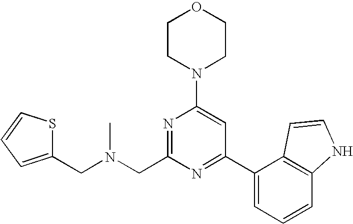 | [4-(1H-Indol-4-yl)-6-morpholin-4-yl- pyrimidin-2-ylmethyl]-methyl-thiophen-2- ylmethyl-amine |
| 38 | 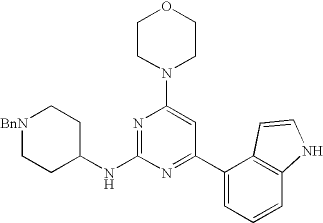 | (1-Benzyl-piperidin-4-yl)-[4-(1H-indol-4- yl)-6-morpholin-4-yl-pyrimidin-2-yl]-amine |
| 39 | 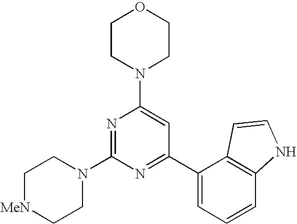 | 4-[4-(4-Methyl-piperazin-1-yl)-6- morpholin-4-yl-pyrimidin-2-yl]-1H-indole |
| 40 | 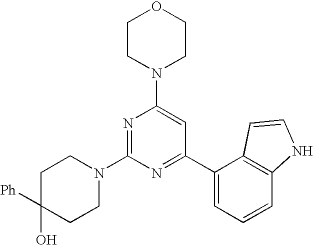 | 1-[2-(1H-Indol-4-yl)-6-morpholin-4-yl- pyrimidin-4-yl]-4-phenyl-piperidin-4-ol |
| 41 | 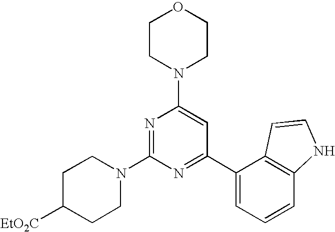 | 1-[2-(1H-Indol-4-yl)-6-morphollin-4-yl- pyrimidin-4-yl]-piperidine-4-carboxylic acid ethyl ester |
| 42 | 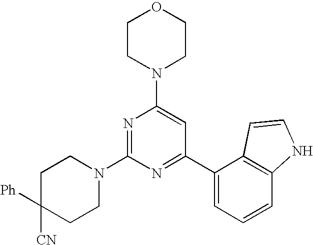 | 1-[2-(1H-Indol-4-yl)-6-morpholin-4-yl- pyrimidin-4-yl]-4-phenyl-piperidine-4- carbonitrile |
| 43 | 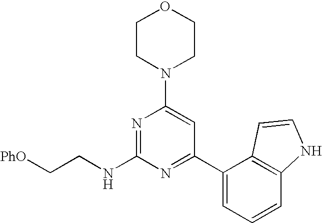 | [2-(1H-Indol-4-yl)-6-morpholin-4-yl- pyrimidin-4-yl]-(2-phenoxy-ethyl)-amine |
| 44 | 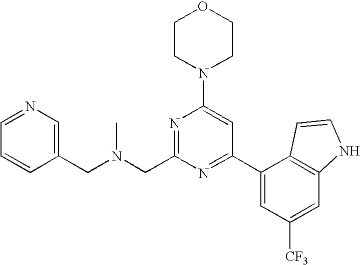 | Methyl-[4-morpholin-4-yl-6-(6- trifluoromethyl-1H-indol-4-yl)-pyrimidin-2- ylmethyl]-pyridin-3-ylmethyl-amine |
| 45 | 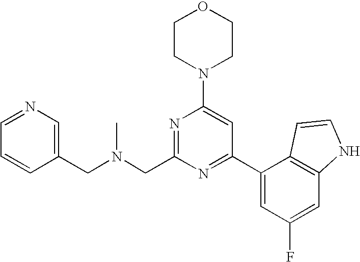 | [4-(6-Fluoro-1H-indol-4-yl)-6-morpholin-4- yl-pyrimidin-2-ylmethyl]-methyl-pyridin-3- ylmethyl-amine |
| 46 | 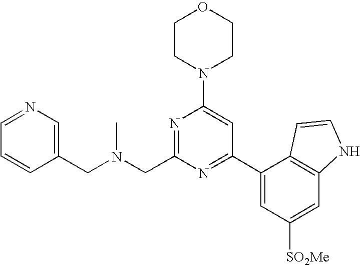 | [4-(6-Methanesulfonyl-1H-indol-4-yl)-6- morpholin-4-yl-pyrimidin-2-ylmethyl]- methyl-pyridin-3-ylmethyl-amine |
| 47 | 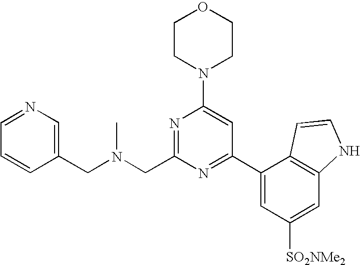 | 4-{2-[(Methyl-pyridin-3-ylmethyl-amino)- methyl]-6-morpholin-4-yl-pyrimidin-4-yl}- 1H-indole-6-sulfonic acid dimethylamide |
| 48 | 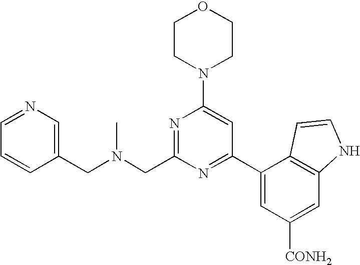 | 4-{2-[(Methyl-pyridin-3-ylmethyl-amino)- methyl]-6-morpholin-4-yl-pyrimidin-4-yl}- 1H-indole-6-carboxylic acid amide |
| 49 | 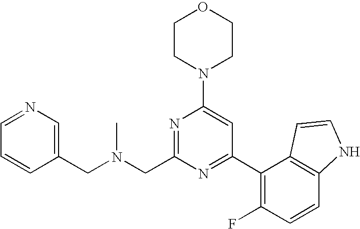 | [4-(5-Fluoro-1H-indol-4-yl)-6-morpholin-4- yl-pyrimidin-2-ylmethyl]-methyl-pyridin-3- ylmethyl-amine |
| 50 | 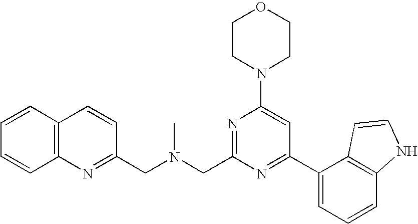 | [4-(1H-Indol-4-yl)-6-morpholin-4-yl- pyrimidin-2-ylmethyl]-methyl-quinolin-2- ylmethyl-amine |
| 51 | 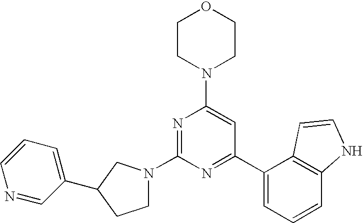 | 1-[2-(1H-indole-4-yl)-6-morpholin-4-yl- pyrimidine-4-yl]-3-pyridin-3-yl-pyrrolidine |

and the pharmaceutically acceptable salts thereof.

Pyrimidines of the invention may be produced by a process which comprises a palladium-mediated (Suzuki-type) cross-coupling reaction, typically as the last step, as the penultimate step or as an intermediate step. When the Suzuki cross-coupling reaction is the last step, a pyrimidine of formula (I) may be produced by a process which comprises treating a compound of formula (IIa) or (IIb):

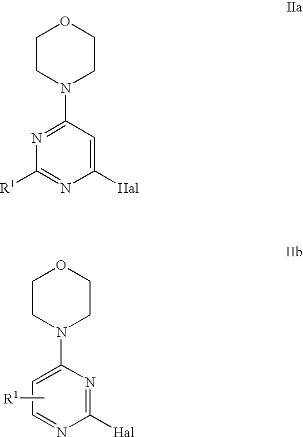

wherein R¹ is as defined above and Hal is a halogen, with a boronic acid or ester thereof of formula R²B(OR¹⁵)₂, in which R² is as defined above and each R¹⁵ is H or C₁-C₆ alkyl or the two groups OR¹⁵ form, together with the boron atom to which they are attached, a pinacolato boronate ester group, in the presence of a Pd catalyst.

The intermediates of formulae (IIa) and (IIb) are known compounds or may be made by routine synthetic chemical techniques. For instance, a compound of formula (IIa) or (IIb) in which R¹ is —(CHR)ₘ—Y—R³ wherein m is 1, Y is a direct bond and R³ is a group —NR⁶R⁷ may be produced by a process which comprises treating a compound of formula (IIIa) or (IIIb):

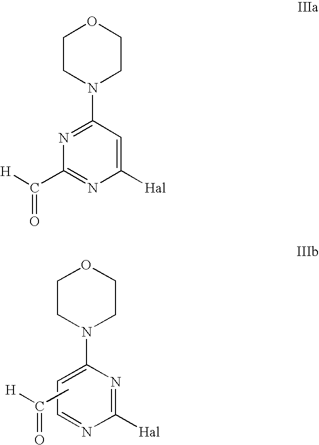

with an amine of formula HNR⁶R⁷ in a solvent under reducing conditions, for instance in the presence of Na(OAc)₃BH or NaBH₄.

A compound of formula (IIa) or (IIb) in which R¹ is —CH₂Y—R³ wherein Y is a direct bond may also be produced by a process which comprises treating a compound of formula (IIIc) or (IIId).

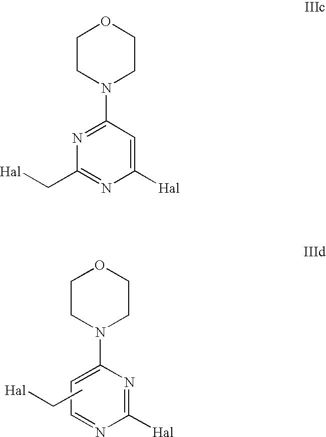

wherein each Hal is halogen, with an amine of formula HNR⁶R⁷ in a solvent, for instance acetonitrile.

A compound of formula (IIa) or (IIb) in which R¹ is —(CHR)ₘ—Y—R³ wherein m is 2 and Y is a direct bond may be produced by a process which comprises reducing a compound of formula (IIIe) or (IIIf):

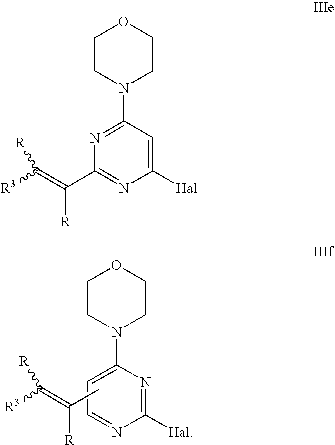

wherein Hal is halogen. The reduction may be performed by any suitable means, for instance hydrogenation in the presence of palladium on carbon.

A compound of formula (IIa) or (IIb) wherein R¹ is —(CR₂)ₘ—Y—R³ wherein Y is —NRC(O)—(CR₂)ₙ— may be produced by a process which comprises treating a compound of formula (IIIg) or (IIIh):

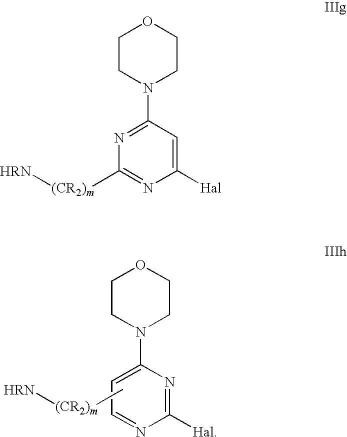

with a carboxylic acid of formula R³—(CR₂)ₙ—COOH in a solvent in the presence of a base and a suitable coupling agent.

When the palladium-mediated Suzuki cross-coupling reaction is the penultimate step, that step may comprise producing an intermediate compound of formula (IIc) or (IId):

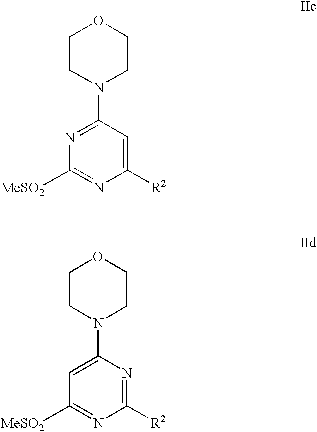

wherein R² is as defined above, by treating a compound of the following formula (IIIi) or (IIIj):

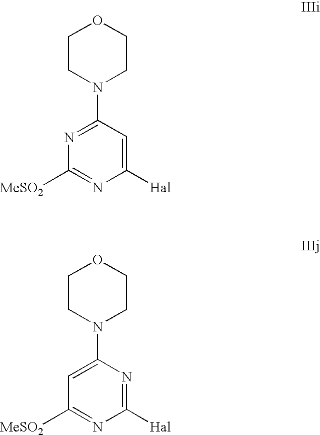

wherein Hal is a halogen with a boronic acid or ester thereof of formula R²B(OR¹⁵)₂, in which R² is as defined above and each R¹⁵ is H or C₁-C₆ alkyl or the two groups OR¹⁵ form, together with the boron atom to which they are attached, a pinacolato boronate ester group, in the presence of a Pd catalyst.

The intermediate compounds of formulae (IIc) and (IId) may be converted to a pyrimidine of formula (I) as defined above in which R¹ is a group —NR⁴R⁵ as defined above, by a process which comprises treating a compound of formula (IIc) or (IId), with an amine of formula HNR⁴R⁵ in a solvent at an elevated temperature.

A compound of formula (IIIi) or (IIIj) may be produced by a process which comprises oxidising a compound of the following formula (IVi) or (IVj):

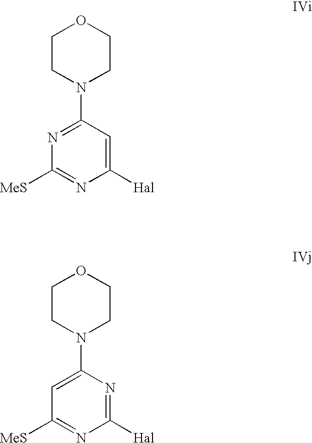

The oxidation may be performed by any suitable method for converting a group —S— to —S(O)₂—

When the Suzuki cross-coupling is an intermediate step, that step may comprise producing an intermediate compound of formula (IIe) or (IIf):

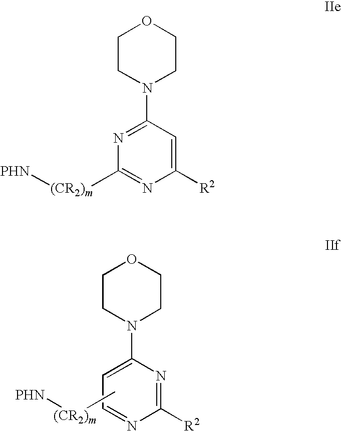

wherein m, R and R² are as defined above and P is an amine protecting group, by treating a compound of the following formula (IIIk) or (IIIl):

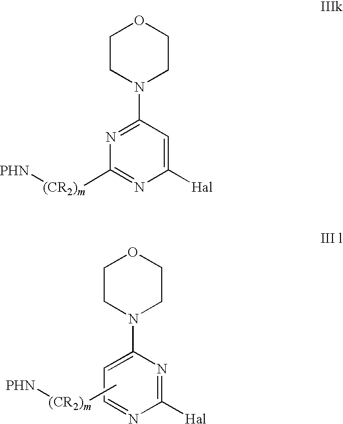

wherein m, R and P are as defined above and Hal is halogen, with a boronic acid or ester thereof of formula R²B(OR¹⁵)₂ wherein R² and R¹⁵ are as defined above, in the presence of a Pd catalyst.

The intermediate compounds of formulae (IIe) and (IIf) may be converted to a pyrimidine of formula (I) as defined above in which R¹ is a group —(CHR)ₘ—Y—R³ wherein Y is —NR—(CHR)ₙ— by removing the protecting group from the compound of formula (IIe) or (IIf) and treating the deprotected amine with a compound of formula R³—Hal wherein Hal is a halogen, typically F, in a solvent in the presence of a base.

Pyrimidines of formula (I) may be converted into pharmaceutically acceptable salts, and salts may be converted into the free compound, by conventional methods. Pharmaceutically acceptable salts include salts of inorganic acids such as hydrochloric acid, hydrobromic acid and sulfuric acid, and salts of organic acids such as acetic acid, oxalic acid, malic acid, methanesulfonic acid, trifluoroacetic acid, benzoic acid, citric acid and tartaric acid. In the case of compounds of the invention bearing a free carboxy substituent, the salts include both the above-mentioned acid addition salts and the salts of sodium, potassium, calcium and ammonium. The latter are prepared by treating the free pyrimidine of formula (I), or the acid addition salt thereof, with the corresponding metal base or ammonia.

Compounds of the present invention have been found in biological tests to be inhibitors of PI3 kinase. The compounds are selective for class Ia PI3 kinases over class Ib. In general the compounds are selective for the p110δ isoform, for instance p110δ over p110δ.

A compound of the present invention may thus be used as an inhibitor of PI3 kinase, in particular of a class Ia PI3 kinase. Accordingly, a compound of the present invention can be used to treat a disease or disorder arising from abnormal cell growth, function or behaviour associated with PI3 kinase. Examples of such diseases and disorders are discussed by Drees et al in Expert Opin. Ther. Patents (2004) 14(5):703-732. These include proliferative disorders such as cancer, immune disorders, cardiovascular disease, viral infection, inflammation, metabolism/endocrine disorders and neurological disorders. Examples of metabolism/endocrine disorders include diabetes and obesity. Examples of cancers which the present compounds can be used to treat include leukaemia, brain tumours, renal cancer, gastric cancer and cancer of the skin, bladder, breast, uterus, lung, colon, prostate, ovary and pancreas.

A compound of the present invention may be used as an inhibitor of PI3 kinase. A human or animal patient suffering from a disease or disorder arising from abnormal cell growth, function or behaviour associated with PI3 kinase, such as an immune disorder, cancer, cardiovascular disease, viral infection, inflammation, a metabolism/endocrine disorder or a neurological disorder, may thus be treated by a method comprising the administration thereto of a compound of the present invention as defined above. The condition of the patient may thereby be improved or ameliorated.

A compound of the present invention can be administered in a variety of dosage forms, for example orally such as in the form of tablets, capsules, sugar- or film-coated tablets, liquid solutions or suspensions or parenterally, for example intramuscularly, intravenously or subcutaneously. The compound may therefore be given by injection or infusion.

The dosage depends on a variety of factors including the age, weight and condition of the patient and the route of administration. Daily dosages can vary within wide limits and will be adjusted to the individual requirements in each particular case. Typically, however, the dosage adopted for each route of administration when a compound is administered alone to adult humans is 0.0001 to 50 mg/kg, most commonly in the range of 0.001 to 10 mg/kg, body weight, for instance 0.01 to 1 mg/kg. Such a dosage may be given, for example, from 1 to 5 times daily. For intravenous injection a suitable daily dose is from 0.0001 to 1 mg/kg body weight, preferably from 0.0001 to 0.1 mg/kg body weight. A daily dosage can be administered as a single dosage or according to a divided dose schedule.

A compound of the invention is formulated for use as a pharmaceutical or veterinary composition also comprising a pharmaceutically or veterinarily acceptable carrier or diluent. The compositions are typically prepared following conventional methods and are administered in a pharmaceutically or veterinarily suitable form. The compound may be administered in any conventional form, for instance as follows:

A) Orally, for example, as tablets, coated tablets, dragees, troches, lozenges, aqueous or oily suspensions, liquid solutions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavouring agents, colouring agents and preserving agents in order to provide pharmaceutically elegant and palatable preparations.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, dextrose, saccharose, cellulose, corn starch, potato starch, calcium phosphate or sodium phosphate; granulating and disintegrating agents, for example, maize starch, alginic acid, alginates or sodium starch glycolate; binding agents, for example starch, gelatin or acacia; lubricating agents, for example silica, magnesium or calcium stearate, stearic acid or talc; effervescing mixtures; dyestuffs, sweeteners, wetting agents such as lecithin, polysorbates or lauryl sulphate. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. Such preparations may be manufactured in a known manner, for example by means of mixing, granulating, tableting, sugar coating or film coating processes.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is present as such, or mixed with water or an oil medium, for example, peanut oil, liquid paraffin, or olive oil.

Aqueous suspensions contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides for example polyoxyethylene sorbitan monooleate.

The said aqueous suspensions may also contain one or more preservatives, for example, ethyl or n-propyl p-hydroxybenzoate, one or more colouring agents, such as sucrose or saccharin.

Oily suspension may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavouring agents may be added to provide a palatable oral preparation. These compositions may be preserved by this addition of an antioxidant such as ascorbic acid. Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of oil-in-water emulsions. The oily phase may be a vegetable oil, for example olive oil or arachis oils, or a mineral oil, for example liquid paraffin or mixtures of these. Suitable emulsifying agents may be naturally-occurring gums, for example gum acacia or gum tragacanth, naturally occurring phosphatides, for example soy bean lecithin, and esters or partial esters derived from fatty acids an hexitol anhydrides, for example sorbitan mono-oleate, and condensation products of the said partial esters with ethylene oxide, for example polyoxyethylene sorbitan monooleate. The emulsion may also contain sweetening and flavouring agents. Syrups and elixirs may be formulated with sweetening agents, for example glycerol, sorbitol or sucrose. In particular a syrup for diabetic patients can contain as carriers only products, for example sorbitol, which do not metabolise to glucose or which only metabolise a very small amount to glucose.

Such formulations may also contain a demulcent, a preservative and flavouring and coloring agents.

B) Parenterally, either subcutaneously, or intravenously, or intramuscularly, or intrasternally, or by infusion techniques, in the form of sterile injectable aqueous or oleaginous suspensions. This suspension may be formulated according to the known art using those suitable dispersing of wetting agents and suspending agents which have been mentioned above. The sterile injectable preparation may also be a sterile injectable solution or suspension in a non-toxic paternally-acceptable diluent or solvent, for example as a solution in 1,3-butane diol.

Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition fatty acids such as oleic acid find use in the preparation of injectables.

C) By inhalation, in the form of aerosols or solutions for nebulizers.

D) Rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

E) Topically, in the form of creams, ointments, jellies, collyriums, solutions or suspesions.

The invention will be further described in the Examples which follow:

### EXAMPLES

### General Synthetic Procedures

The following general schemes 1 to 10 are referred to in the Reference Examples as Examples which follow:

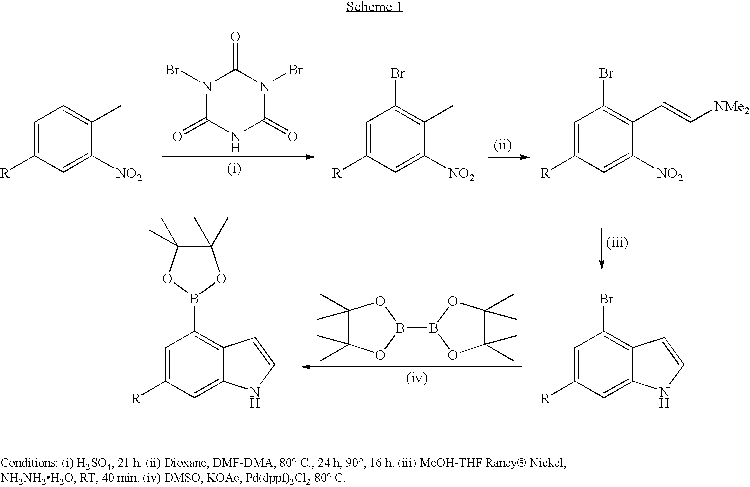

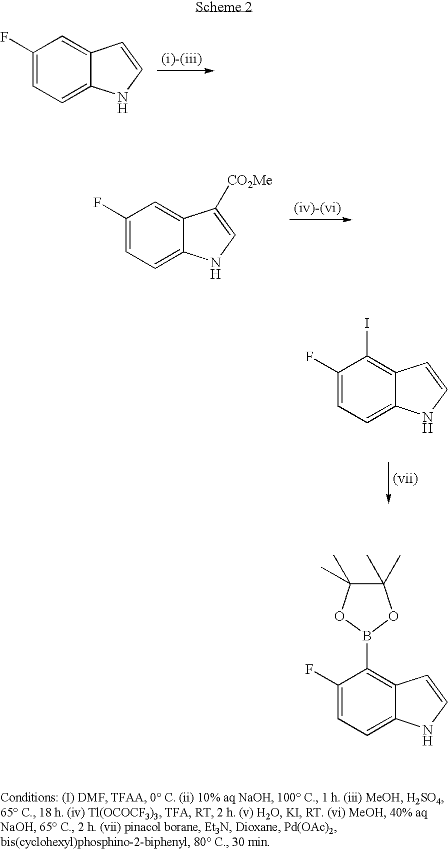

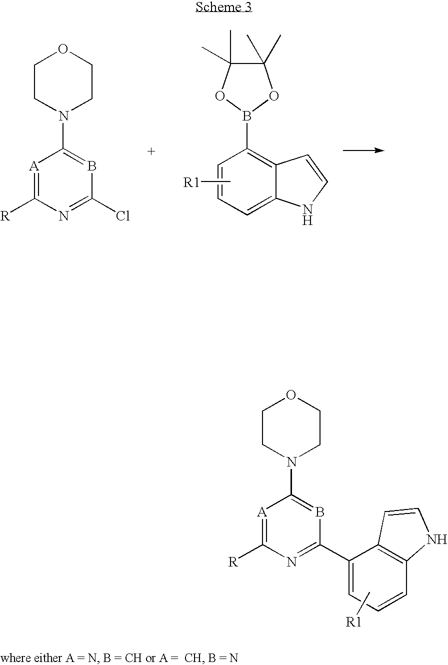

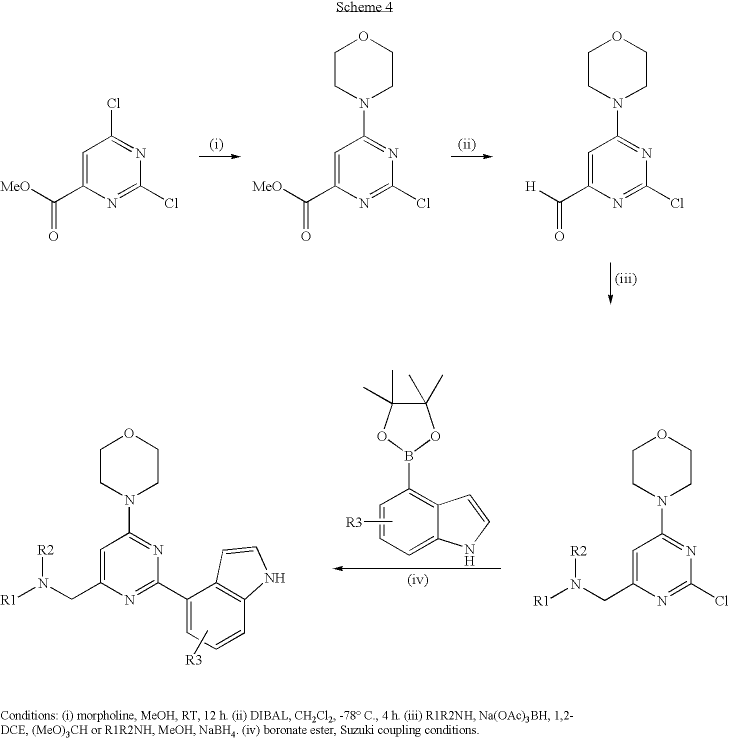

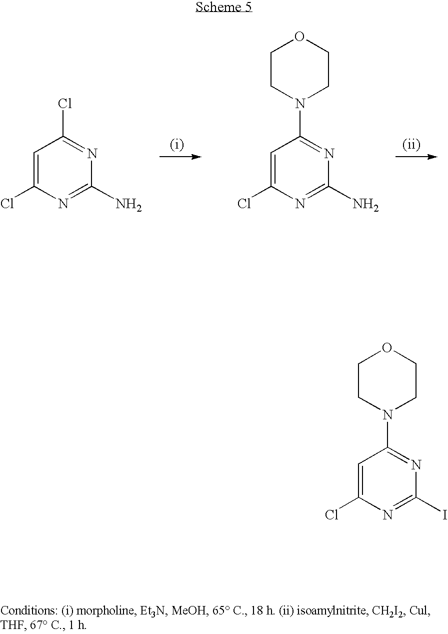

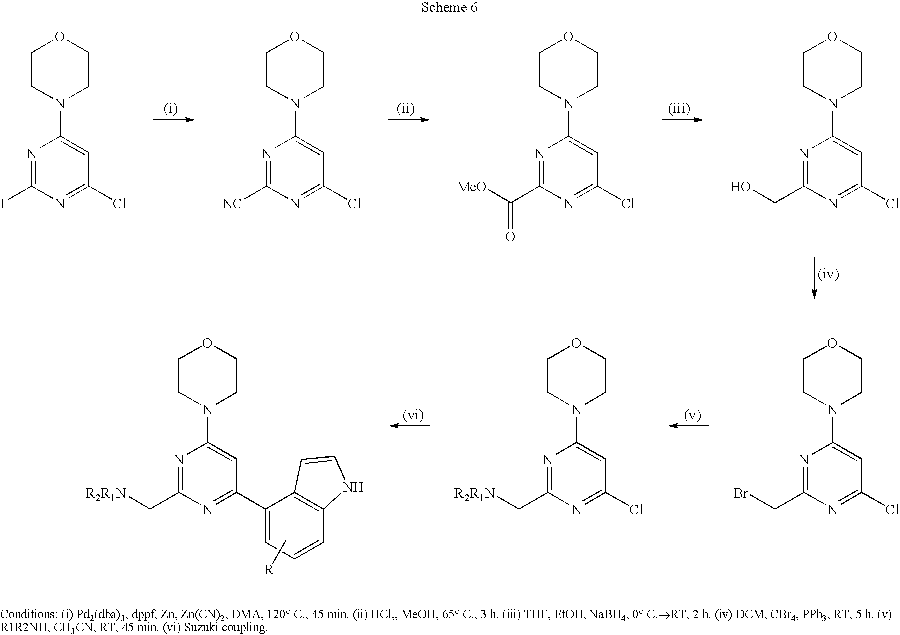

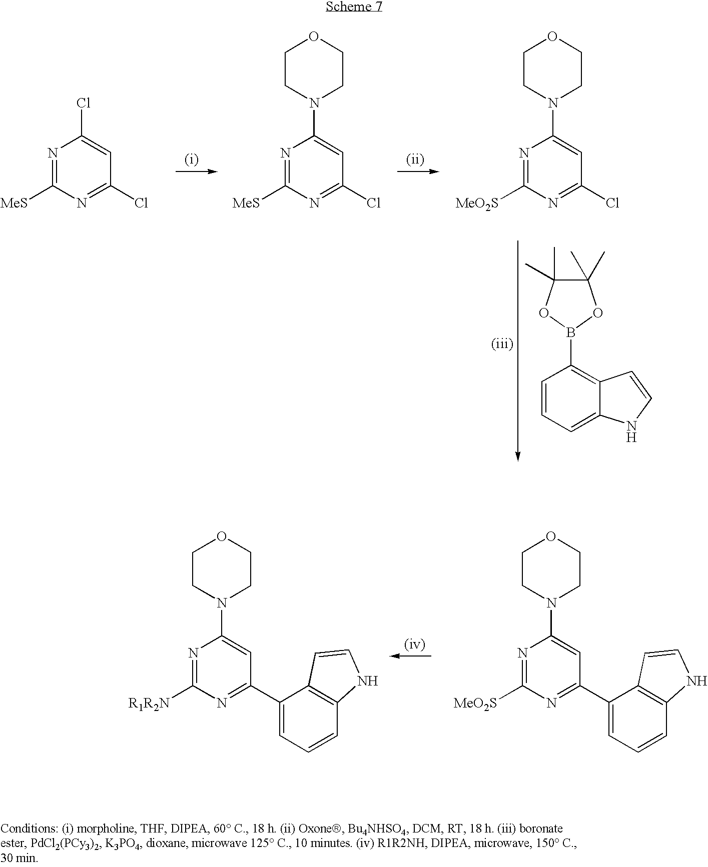

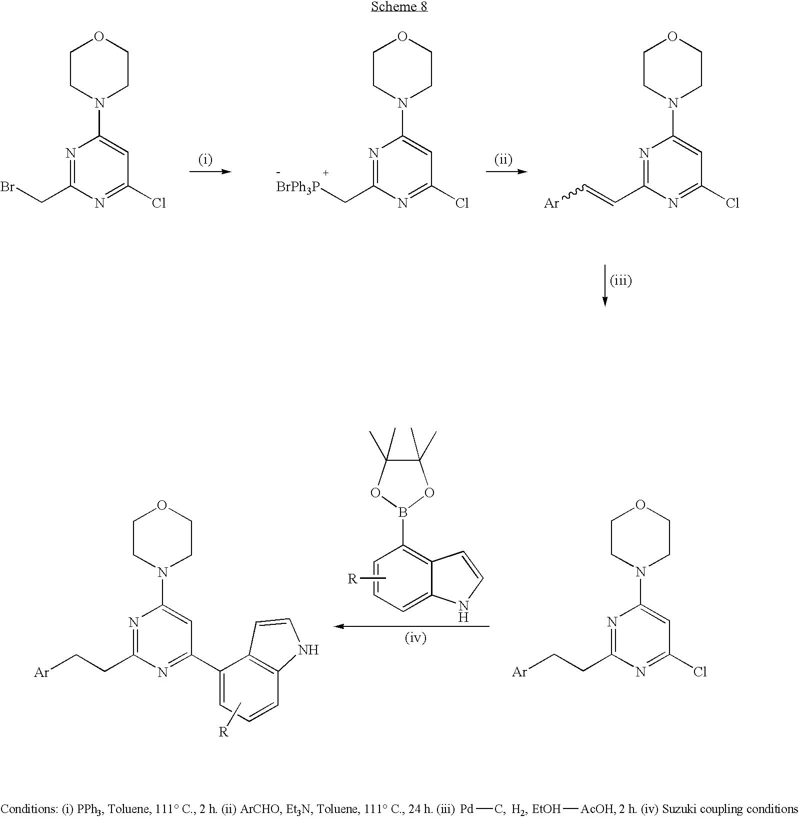

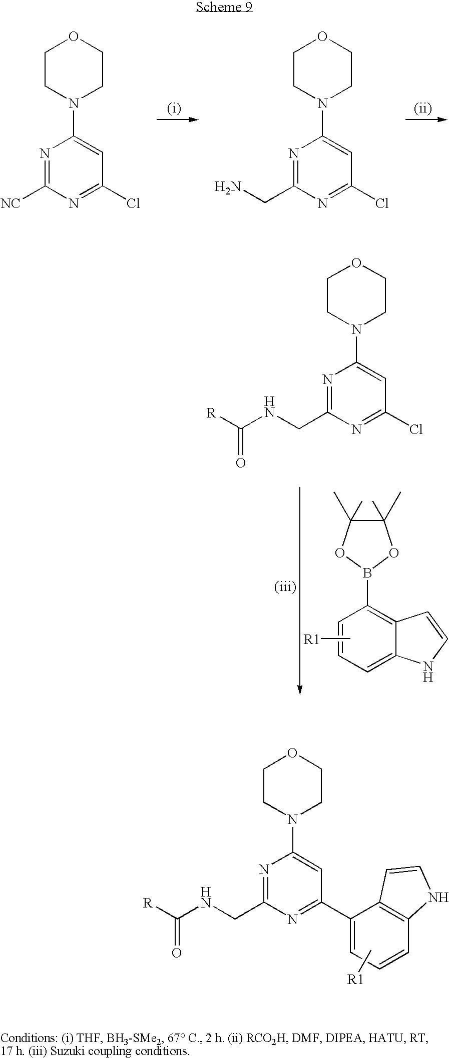

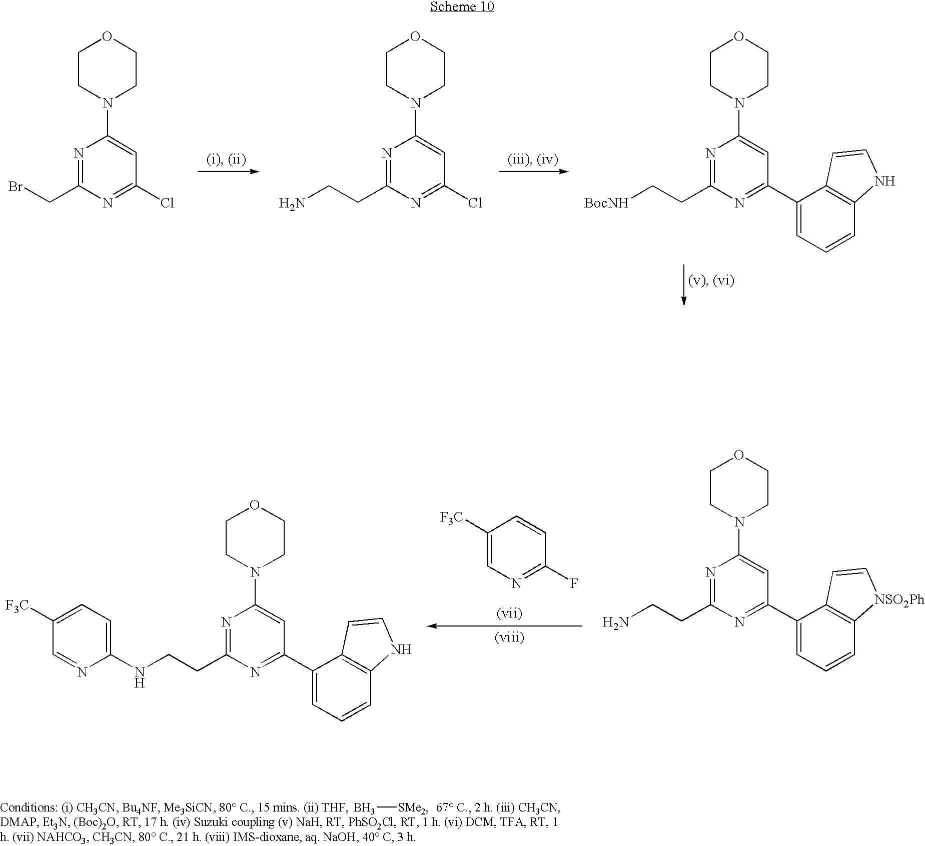

### General Experimental Details:

### NMR Spectrometry:

NMR spectra were obtained on a Varian Unity Inova 400 spectrometer with a 5 mm inverse detection triple resonance probe operating at 400 MHz or on a Bruker Avance DRX 400 spectrometer with a 5 mm inverse detection triple resonance TXI probe operating at 400 MHz or on a Bruker Avance DPX 400 spectrometer with a 5 mm ¹H/¹³C Dual autotune probe operating at 400 MHz for ¹H or on a Bruker Avance DPX 300 spectrometer with a standard 5 mm dual frequency probe operating at 300 MHz. Shifts are given in ppm relative to tetramethylsilane @ 303K.

### Purification by Column Chromatography:

Compounds purified by column chromatography were purified using silica gel or Isolute® cartridge or Redisep® cartridge, eluting with gradients from 100-0 to 0-100% of cyclohexane/EtOAc, or from 100-0 to 0-100% pentane/EtOAc or from 100-0 to 70-30% DCM/MeOH (with or without the addition of NH₃ 0.1%). ‘Silica gel’ refers to silica gel for chromatography, 0.035 to 0.070 mm (220 to 440 mesh) (e.g. Fluka silica gel 60), and an applied pressure of nitrogen up to 10 p.s.i accelerated column elution. Where thin layer chromatography (TLC) has been used, it refers to silica gel TLC using plates, typically 3×6 cm silica gel on aluminium foil plates with a fluorescent indicator (254 nm), (e.g. Fluka 60778).

### Purification by Preparative HPLC:

Compounds purified by preparative HPLC were purified using either conditions A: Waters)(Bridge Prep Phenyl column (150×19 mm i.d. column with 5 μm particle size, PDA/MS detetction, flow 21.25 ml/min), eluting with gradients from 95-5% to 5-95% water/acetonitrile containing 0.1% dimethylethylamine; or conditions B: C18-reverse-phase column (100×22.5 mm i.d. Genesis column with 7 μm particle size, UV detection at 230 or 254 nm, flow 5-15 mL/min), eluting with gradients from 100-0% to 0-100% water/acetonitrile or water/MeOH containing 0.1% TFA; or conditions C: Phenyl-Hexyl column (250×21.2 mm i.d. Gemini column with 5 μm particle size, UV detection at 230 or 254 nm, flow 5-20 mL/min), eluting with gradients from 100-0% to 0-100% water/acetonitrile or water/MeOH containing 0.1% TFA or water/acetonitrile containing 0.1% formic acid. When using conditions B or C the free base was liberated by partitioning between EtOAc and a sat. solution of sodium bicarbonate. The organic layer was dried (MgSO₄) and concentrated in vacuo. Alternatively, the free base was liberated by passing through an Isolute® SCX-2 cartridge, eluting with NH₃ in methanol.

Abbreviations used in the experimental section:
aq.=aqueous
BOC=t-Butoxycarbonyl
bs=broad singlet (NMR)
Cs₂CO₃=cesium carbonate
d=doublet (NMR)
DCM=dichloromethane
DCE=1,2-dichloroethane
DIPEA=diisopropylethylamine
DMA=dimethylacetamide
DMAP=dimethylaminopyridine
DMF=dimethylformamide
DMSO=dimethylsulfoxide
eq.=equivalents
EtOAc=ethyl acetate
EtOH=ethanol
h=hour(s)
HATU=O-(7-Azabenzotriazol-1-yl)-N,N,N′,N′-tetramethyluronium hexafluorophosphate
HCl=hydrochloric acid
H₂O=water
HPLC=high pressure liquid chromatography
IMS=industrial methylated spirit
iPrOH=isopropanol
LCMS=liquid chromatography mass spectrometry
M=molar
m=multiplet (NMR)
MeOH=methanol
mg=milligram
MgSO₄=magnesium sulphate
min=minute(s)
mL=millilitre
Na₂CO₃=sodium carbonate
NaHCO₃=sodium hydrogen carbonate
NaOH=sodium hydroxide
Na₂SO₄=sodium sulfate
NMR=nuclear magnetic resonance
q=quartet (NMR)
Rt=retention time
RT=room temperature
sat=saturated
t=triplet (NMR)
TFA=trifluoroacetic acid
THF=tetrahydrofuran
TLC=thin layer chromatography

### Reference Example 1

### Formation of Boronate Ester

The boronate ester product of the final step of scheme 1 above was prepared as follows. To a solution of halide (1 eq.) and bis(pinacolato)diboron (1.3 eq.) in DMSO were added KOAc (3 eq.) and [1,1′-bis(diphenylphosphine)ferrocene]-dichloropalladium (0.05 eq.). The mixture was heated at 90° C. until completion of the reaction. The reaction mixture was partioned between EtOAc and H₂O. The organic layer was washed successively with H₂O and brine, dried over Na₂SO₄ and evaporated to dryness. The resultant residue was then purified by column chromatography.

### Reference Example 2

### Suzuki Coupling

The Suzuki coupling depicted in general terms in scheme 3 above was performed using one of the following three synthetic strategies:

### Method A.

A mixture of 2-chloro-pyrimidine (1 eq.), Na₂CO₃ (2 eq.), indole boronate ester (1.5 eq.) and bis(triphenylphosphine)palladium (II) chloride (0.1 eq.) in acetonitrile/water (2:1) was heated at 140° C. for 20-50 min in a microwave reactor (Smith synthetiser or CEM Discover). The resulting mixture was diluted with water then extracted with ethyl acetate. The combined organic extracts were dried (Na₂SO₄), filtered and concentrated then purified by either preparative HPLC or column chromatography to give the desired product.

### Method B.

A mixture of 2-chloro-pyrimidine (1 eq.), Cs₂CO₃ (1.5 eq.), indole boronate ester (1.2 eq.) and tetrakis(triphenylphosphine)palladium (0.05 eq.) in dioxane/water (3:1) was heated at 125° C., for 10-30 min in a microwave reactor (Smith synthetiser). The resulting mixture was diluted with water then extracted with ethyl acetate. The combined organic extracts were dried (MgSO₄), filtered and concentrated then purified by either preparative HPLC or column chromatography to give the desired product.

### Method C.

A mixture of 2-chloro-pyrimidine (1 eq.), Cs₂CO₃ (1.5 eq.), indole boronate ester (1.2 eq.) and tetrakis(triphenylphosphine)palladium (0.05 eq.) in dioxane/water (3:1⁻) was heated at 125° C., for 10-30 min in a microwave reactor (Smith synthetiser). The resulting mixture was loaded onto Isolute® SCX-2 cartridge, washed with MeOH then eluted with 2 M NH₃ in MeOH. The resulting residue was then purified by either preparative HPLC or column chromatography to give the desired product.

### Method D.

A stirred mixture of chloropyrimidine (1 eq.), indole boronate ester (1.4 eq.) and PdCl₂(PCy₃)₂ (0.02 eq.) in K₃PO₄ (0.5 ml of a 1.27 M aqeuous solution) and dioxane (1.0 mL) was heated at 140° C. in a microwave for 30 min. The product was purified by catch-and-release using an Isolute SCX-2 cartridge followed by flash chromatography to give the desired product.

### Reference Example 3

### Amination Procedures

The reductive aminations depicted in the above schemes were performed using one of the following three synthetic strategies:

### Method E

To the pyrimidine aldehyde (1 eq.) in 1,2-DCE (7 mL) was added amine (2 eq.) and trimethylorthoformate (10 eq.) and the mixture stirred for 1 hour at RT. Sodium triacetoxyborohydride (2.3 eq.) was added portionwise over 10 minutes and the reaction mixture stirred for 18 h. The mixture was then partitioned between DCM and aqueous NaHCO₃ solution. The combined organic layers were washed with brine, separated and dried and the crude material purified by column chromatography to give the desired compound.

### Method F

To a solution of the pyrimidine aldehyde (1 eq.) in methanol (10 mL) was added amine (1.1 eq.) and the mixture stirred at RT for 12 hours. Sodium borohydride (1.8 eq.) was then added in a single portion and the mixture stirred for 2.5 hours. The reaction mixture was then evaporated onto silica and purified by column chromatography to give the desired compound.

### Method G:

General procedure for the microwave-assisted displacement of 4-(2-methane-sulfonyl-6-morpholin-4-yl-pyrimidin-4-yl)-1H-indole with amines:

A stirred mixture of 4-(2-methanesulfonyl-6-morpholin-4-yl-pyrimidin-4-yl)-1H-indole (72 mg; 0.20 mmol), amine (10 eq.) and DIPEA (0.1 ml; 0.58 mmol) in dioxane (0.3 mL) was heated in a microwave at 150° C. for 30 min. The reaction mixture was purified directly by flash chromatography or preparative LCMS.

### Reference Example 4

### 4,N,N-Trimethyl-3-nitro-benzenesulfonamide

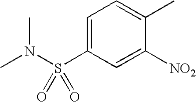

To a solution of dimethylamine in H₂O (40% w/w, 15.0 mL, 120 mmol) at 0° C. was added a solution of 4-methyl-3-nitro-benzenesulfonyl chloride (9.42 g, 40 mmol) in DCM (60 mL) over 30 min. The resulting mixture was stirred at 0° C. for 30 min before being allowed to warm to RT and stirred overnight. The reaction mixture was diluted with H₂O (100 mL) and DCM (40 mL), and the layers were separated. The organic layer was washed in succession with water, HCl (aq., 0.1 M) and brine before being dried over Na₂SO₄ and evaporated to dryness to give the title compound as a pale yellow solid (9.13 g, 94%).

[M+H]⁺ 244.9

### Reference Example 5

### 3-Bromo-4,N,N-trimethyl-5-nitro-benzenesulfonamide

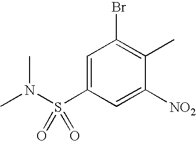

To a solution of 4-N,N-trimethyl-3-nitro-benzenesulfonamide (8.57 g, 34.7 mmol) in concentrated sulfuric acid (80 mL) was added 1,3-dibromo-[1,3,5]triazinane-2,4,6-trione (5.97 g, 20.8 mmol) and the orange reaction mixture was stirred at RT for 16 h. A further 2 g of 1,3-dibromo-[1,3,5]triazinane-2,4,6-trione was added and stirring continued for 5 h. The reaction mixture was then poured onto ice and water and stirred for 15 min. The resulting milky/white solid was filtered and washed with H₂O, before being dissolved in EtOAc. The organic layer was dried over Na₂SO₄ and evaporated to dryness to give the title compound as a white solid (10.41 g, 93%).

[M+H]⁺ 323.1 (⁷⁹Br) 325.0 (⁸¹Br)

### Reference Example 6

### 1-Bromo-5-methanesulfonyl-2-methyl-3-nitro-benzene

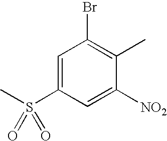

Prepared according to the method used in the preparation of 3-bromo-4-N,N-trimethyl-5-nitro-benzenesulfonamide using 4-methanesulfonyl-1-methyl-2-nitro-benzene in place of 4-N,N-trimethyl-3-nitro-benzenesulfonamide. The title compound was obtained as a white solid (17.0 g, 85%).

[M+H]⁺ 294.1 (⁷⁹Br) 296.0 (⁸¹Br)

### Reference Example 7

### 1-Bromo-5-fluoro-2-methyl-3-nitro-benzene

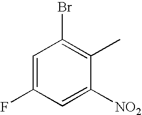

Prepared according to the method used in the preparation of 3-bromo-4-N,N-trimethyl-5-nitro-benzenesulfonamide using 4-fluoro-1-methyl-2-nitro-benzene in place of 4-N,N-trimethyl-3-nitro-benzenesulfonamide. The title compound was obtained as a yellow solid (68.0 g, 79%).

NMR δ_{H} (300 MHz, CDCl₃) 2.59 (s, 3H), 7.50 (dd, J=2.8, 7.6, 1H) and 7.58 (dd, J=2.9, 7.4, 1H).

### Reference Example 8

### 4-Bromo-1H-indole-6-sulfonic acid dimethylamide

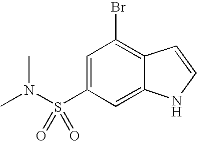

To a solution of 3-bromo-4-N,N-trimethyl-5-nitro-benzenesulfonamide (9.15 g, 28.3 mmol) in dioxane (60 mL) was added DMF-DMA (11.3 mL, 84.9 mmol). The deep red reaction mixture was heated at 80° C. for 24 h followed by heating at 90° C. for 16 h. The mixture was cooled to RT and concentrated to 50% of the volume, poured into H₂O and extracted into EtOAc. The organic layer was isolated and washed with H₂O, then brine, dried over Na₂SO₄, and evaporated to dryness to give 3-bromo-4-(2-dimethylamino-vinyl)-N,N-dimethyl-5-nitro-benzenesulfonamide as a red solid (10.4 g, 91%). To a suspension of the amide (10.4 g, 25.7 mmol) and Raney®-Nickel (suspension in H₂O, 20 mL) in MeOH:THF (1:1, 200 mL) was added hydrazine monohydrate (1.9 mL, 38.6 mmol) at 0° C. and the mixture stirred at RT for 40 min. The reaction mixture was then filtered through Celite and the filter cake washed with EtOAc and H₂O. The aqueous layer was isolated and then extracted with EtOAc. The combined organic layers were washed with H₂O, followed by brine, dried over Na₂SO₄ then evaporated to dryness. The resulting pink solid was purified by column chromatography, and subsequently recrystallised from iPrOH and EtOH to give the title compound as a white solid (3.5 g, 41%).

NMR δ_{H} (400 MHz, CDCl₃) 2.72 (s, 6H), 6.70 (m, 1H), 7.49 (apparent t, J=2.7, 1H), 7.68 (d, J=1.1, 1H), 7.94 (m, 1H) and 9.04 (bs, 1H).

### Reference Example 9

### 4-Bromo-6-methanesulfonyl-1H-indole

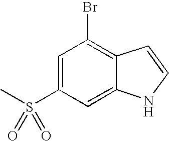

Prepared according to the method used in the preparation of 4-bromo-1H-indole-6-sulfonic acid dimethylamide using 1-bromo-5-methanesulfonyl-2-methyl-3-nitro-benzene in place of 3-bromo-4-N,N-trimethyl-5-nitro-benzenesulfonamide. The title compound was obtained as a white solid (1.8 g, 76%).

NMR δ_{H} (300 MHz, CDCl₃) 3.11 (s, 3H), 6.70 (m, 1H), 7.52 (dd, J=2.5, 3.0, 1H), 7.81 (d, J=1.5, 1H), 8.10 (dd, J=1.0, 1.5, 1H) and 9.34 (bs, 1H).

### Reference Example 10

### 4-Bromo-6-fluoro-1H-indole

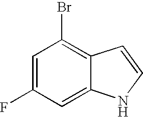

Prepared according to the method used in the preparation of 4-bromo-1H-indole-6-sulfonic acid dimethylamide using 1-bromo-5-fluoro-2-methyl-3-nitro-benzene in place of 3-bromo-4-N,N-trimethyl-5-nitro-benzenesulfonamide. The title compound was obtained as a white solid (6.06 g, 33%).

NMR δ_{H} (300 MHz, CDCl₃) 6.57 (apparent t, J=2.7, 1H), 7.04 (dd, J=2.1, 9.1, 1H), 7.12 (dd, J=2.1, 9.1, 1H), 7.20-7.25 (m, 1H) and 8.25 (s, 1H).

### Reference Example 11

### 2-Methyl-1,3-dinitro-5-trifluoromethyl-benzene

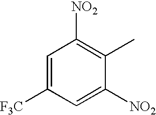

To a solution of 4-methylbenzo-trifluoride (9.51 g, 59.4 mmol) in concentrated sulphuric acid (120 mL) was added potassium nitrate (15.0 g, 0.149 mol) and the resulting mixture stirred at RT for 16 h. The reaction mixture was poured onto ice and water then extracted into EtOAc. The organic layer was washed successively with H₂O and brine, dried over Na₂SO₄ and evaporated to dryness to give the title compound as a yellow solid (13.84 g, 93%)

NMR δ_{H} (400 MHz, CDCl₃) 2.67 (s, 3H) and 8.27 (s, 2H).

### Reference Example 12

### 6-Trifluoromethyl-1H-indol-4-ylamine

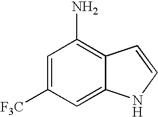

Prepared according to the method used in the preparation of 4-bromo-1H-indole-6-sulfonic acid dimethylamide using 2-methyl-1,3-dinitro-5-trifluoromethyl-benzene in place of 3-bromo-4,N,N-trimethyl-5-nitro-benzenesulfonamide. The title compound was obtained as a white solid (10.7 g, 99%).

[M+H]⁺ 201.1

### Reference Example 13

### 4-Iodo-6-trifluoromethyl-1H-indole

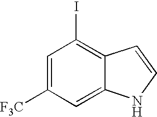

To a suspension of 6-trifluoromethyl-1H-indol-4-ylamine (10.7 g, 53.4 mmol) in HCl (aq., 15%, 240 mL) was added a solution of sodium nitrite (5.52 g, 80.1 mmol) in H₂O (10 mL) slowly at 0° C. The reaction mixture was stirred at RT for 1 h before a solution of sodium tetrafluoroborate (23.5 g, 0.214 mol) in H₂O (30 mL) was added. After stirring for 15 min, the resulting precipitate was collected by filtration and washed with a sodium tetrafluoroborate solution (aq., sat) before dissolving in acetonitrile (100 mL). This solution was added slowly to a suspension of sodium iodide (24.0 g, 0.160 mol) in acetonitrile (100 mL) and the mixture stirred at RT for 16 h. The reaction mixture was concentrated to 30% of the volume and partioned between EtOAc and H₂O. The organic layer was isolated then washed in succession with sodium thiosulfate, H₂O and brine, dried over Na₂SO₄ and evaporated to dryness. The resulting brown oil was purified by column chromatography to give the title compound (9.77 g, 59%).

[M−H]⁻ 310.1

### Reference Example 14

### 4-(4,4,5,5-Tetramethyl-[1,3,2]dioxaborolan-2-yl)-1H-indole-6-carboxylic acid amide

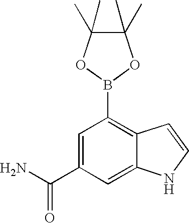

A solution of 4-bromo-1H-indole-6-carbonitrile (1 g, 4.50 mmol) in methanol (10 mL) was treated with 30% aqueous hydrogen peroxide (2.7 mL, 4.95 mmol) and a 1 M aqueous sodium hydroxide solution (5 mL) then heated at 40° C. for 1 h. The reaction mixture was cooled, treated with water and cooled in an ice-bath. The resulting precipitate was collected by filtration, washed with water and dried in vacuo to obtain 4-bromo-1H-indole-6-carboxylic acid amide (1.05 g, 97%), which was transformed into the title boronic ester by the general method (Scheme 1) (0.80 g, 67%).

NMR δ_{H} (300 MHz, DMSO-d₆) 1.35 (s, 12H), 6.78 (m, 1H), 7.10 (s, 1H), 7.51-7.54 (m, 1H), 7.94-7.97 (m, 2H), 8.06 (s, 1H) and 11.40 (bs, 1H).

### Reference Example 15

### 5-Fluoro-4-(4,4,5,5-tetramethyl-[1,3,2]dioxaborolan-2-yl)-1H-indole

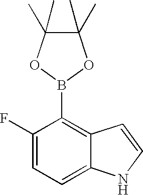

A solution of 5-fluoroindole (5 g, 37.0 mmol) in DMF (40 mL) was treated at 0° C. with trifluoroacetic anhydride (6.1 mL, 42.6 mmol). After 30 min, the reaction was poured into water and the resulting precipitate collected by filtration, washed with water, then dried in vacuo. The solid was then dissolved in 10% aqueous NaOH (200 mL) and heated at reflux for 1 h. The reaction mixture was then cooled, washed with dichloromethane and acidified with aqueous HCl. The resulting white precipitate was collected by filtration, washed with water, taken up in dichloromethane, washed with water, dried (MgSO₄) and evaporated in vacuo. The resulting material (5 g, 75%) was dissolved in methanol (80 mL) and treated with concentrated sulphuric acid (2 mL) then heated at reflux overnight. The reaction was cooled and the resulting precipitate collected, washed with water and evaporated in vacuo to give 5-fluoro-1H-indole-3-carboxylic acid methyl ester as a peach-coloured solid (4.5 g, 83%).

A solution of thallium tris(trifluoroacetate) (8.45 g, 15.6 mmol) in TFA (35 mL) was added to a solution of 5-fluoro-1H-indole-3-carboxylic acid methyl ester (2 g, 10.4 mmol) in TFA (10 mL) at room temperature and stirred for 2 h. The reaction mixture was evaporated in vacuo and the resulting residue suspended in water (25 mL) before being treated with a solution of potassium iodide (5.2 g, 31.3 mmol) in water (50 mL). The reaction mixture was treated with dichloromethane (100 mL) and methanol (5 mL) and the resulting precipitate removed by filtration through celite. The organic layer was separated, washed successively with sodium thiosulfate solution and brine, then dried (MgSO₄) and evaporated in vacuo. The resultant material was dissolved in methanol (60 mL) and treated with 40% aqueous NaOH solution (60 mL) then refluxed for 2 h. The reaction mixture was cooled and extracted with DCM/MeOH (ratio 95:5), dried (MgSO₄), filtered and evaporated in vacuo to give a crude solid. Purification by column chromatography gave 5-fluoro-4-iodo-1H-indole as a pale brown solid (1.05 g, 39%).

NMR δ_{H} (300 MHz, CDCl₃) 6.49-6.52 (m, 1H), 6.95 (apparent dt, J=0.4, 8.6, 1H), 7.26-7.33 (m, 2H) and 8.35 (s, 1H).

A solution of 5-fluoro-4-iodo-1H-indole (261 mg, 1.0 mmol) in dioxane (1 mL) was treated with triethylamine (0.2 mL, 1.4 mmol), palladium acetate (4.5 mg, 0.02 mmol) and bis(cyclohexyl)phosphino-2-biphenyl (28 mg, 0.08 mmol) then heated to 80° C. A solution of pinacolborane (1 M in THF, 2.66 mL, 2.66 mmol) was added via syringe. After 30 min, the reaction mixture was cooled, then diluted with water (10 mL) and DCM (10 mL). The resulting mixture was passed through a phase separation cartridge, and the dichloromethane layer was evaporated in vacuo to obtain the title compound which was used without further purification.

### Reference Example 16

### 4-Chloro-6-morpholin-4-yl-pyrimidine-2-carbonitrile

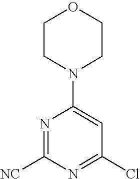

To a mixture of 4-(6-chloro-2-iodo-pyrimidin-4-yl)-morpholine (2 g, 6.14 mmol), tris-(dibenzylideneacetone)dipalladium (113 mg, 0.123 mmol), 1,1′-bis(diphenylphosphino)-ferrocene (136 mg, 0.246 mmol), zinc dust (48 mg, 0.737 mmol) and zinc cyanide (433 mg, 3.69 mmol) was added DMA (20 mL). The reaction mixture was degassed then heated at 120° C. for 45 min. The reaction mixture was left to cool to RT and diluted with EtOAc and aqueous ammonia (33%). The organic layer was isolated, washed with brine, then dried (Na₂SO₄), and concentrated in vacuo. The resultant residue was purified by column chromatography to afford the title compound as a yellow solid (910 mg, 66%).

[M]⁺ 224.6

### Reference Example 17

### C-(4-Chloro-6-morpholin-4-yl-pyrimidin-2-yl)-methylamine

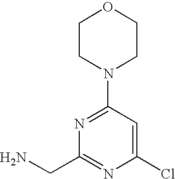

To a solution of 4-chloro-6-morpholin-4-yl-pyrimidine-2-carbonitrile (0.1 g, 0.45 mmol) in dry THF (10 mL) at 55° C. was added borane dimethylsulfide complex (2 M in THF, 1 mL, 2 mmol) and the mixture was heated at a gentle reflux for 2 h. The mixture was cooled to RT, then to 0° C. in an ice bath before. MeOH (2 mL) and an aqueous solution of HCl (1 M, 0.5 mL) were added. The mixture was stirred for 30 min and the solvent was removed in vacuo. The resultant residue was treated with a sat. aqueous solution of NaHCO₃ and extracted several times with DCM. The combined organic layers were washed with brine, dried (Na₂SO₄) and concentrated in vacuo. The resultant residue was purified by passing through an Isolute® SCX-2 cartridge, eluting with 2 M NH₃ in MeOH to give the title compound as a gum (28 mg, 28%).

[M+H]⁺ 228.9

### Reference Example 18

### 4-Chloro-6-morpholin-4-yl-pyrimidine-2-carboxylic acid methyl ester

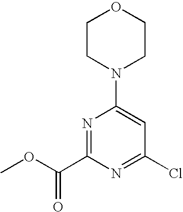

4-Chloro-6-morpholin-4-yl-pyrimidine-2-carbonitrile (1 g, 4.46 mmol) was dissolved in a sat. solution of HCl in MeOH (40 mL) and heated at reflux for 3 h. The solvent was concentrated in vacuo and the resultant residue was dissolved in DCM, then washed with an aqueous sat. solution of NaHCO₃. The organic layer was isolated, dried (MgSO₄) and concentrated in vacuo to yield the title compound as a yellow solid (851 mg, 74%).

[M]⁺ 257.7

### Reference Example 19

### (4-Chloro-6-morpholin-4-yl-pyrimidin-2-yl)-methanol

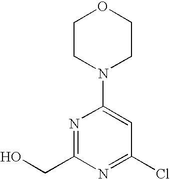

To a suspension of 4-chloro-6-morpholin-4-yl-pyrimidine-2-carboxylic acid methyl ester (5.2 g, 20 mmol) in THF (150 mL) and EtOH (50 mL) under an argon atmosphere at 0° C. was added sodium borohydride (1.53 g, 40 mmol). The reaction mixture was stirred at RT for 2 h. The solvent was removed by evaporation, then the resultant residue was dissolved in EtOAc and a sat. solution of ammonium chloride was added. The resulting mixture was stirred for 30 min. The aqueous layer was separated and extracted with EtOAc. The combined organic layers were washed with brine, dried (Na₂SO₄), and concentrated in vacuo to afford the title compound as an orange solid (3.93 g, 86%).

[M+H]⁺ 229.7

### Reference Example 20

### 4-(2-Bromomethyl-6-chloro-pyrimidin-4-yl)-morpholine

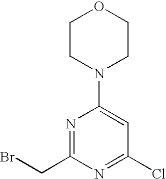

To a solution of (4-chloro-6-morpholin-4-yl-pyrimidin-2-yl)-methanol (3.73 g, 16 mmol) in DCM (60 mL) under argon was added carbon tetrabromide (6.47 g, 19 mmol) and triphenylphosphine (5.54 g, 21 mmol). The reaction mixture was stirred at RT for 5.5 h. The mixture was concentrated to give a brown gum which was purified by column chromatography to yield the title compound as an orange solid (3.79 g, 81%).

[M+H]⁺ 292.0, 294.0

### Reference Example 21

### (4-Chloro-6-morpholin-4-yl-pyrimidin-2-yl)-acetonitrile

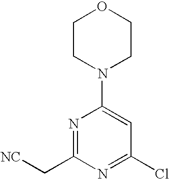

To a solution of 4-(2-bromomethyl-6-chloro-pyrimidin-4-yl)-morpholine (1.5 g, 5.13 mmol) in anhydrous acetonitrile (50 mL) were added a solution of tetrabutylammonium fluoride in THF (1 M, 7.7 mL, 7.7 mmol) and trimethylsilyl cyanide (1.03 mL, 7.72 mmol). The resulting mixture was heated at reflux for 15 min. The reaction mixture was cooled to RT and an aqueous solution of ammonia (33%, 10 mL) was carefully added. The reaction mixture was extracted with DCM and the organic layer was isolated, then washed with brine, dried (MgSO₄) and concentrated in vacuo. The resultant residue was purified by column chromatography to give the title compound as a white solid (0.88 g, 72%).

[M+H]⁺ 239.1

### Reference Example 22

### 2-(4-Chloro-6-morpholin-4-yl-pyrimidin-2-yl)-ethylamine

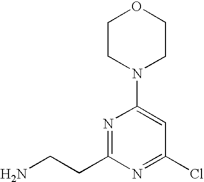

Prepared according to the method used in the preparation of C-(4-chloro-6-morpholin-4-yl-pyrimidin-2-yl)-methylamine using (4-chloro-6-morpholin-4-yl-pyrimidin-2-yl)-acetonitrile in place of 4-chloro-6-morpholin-4-yl-pyrimidine-2-carbonitrile. The title compound was obtained as pale yellow oil (0.1 g, 30%).

[M+H]⁺ 243.2

### Reference Example 23

### [2-(4-Chloro-6-morpholin-4-yl-pyrimidin-2-yl)-ethyl]-carbamic acid tent-butyl ester

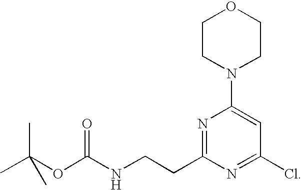

To a solution of 2-(4-chloro-6-morpholin-4-yl-pyrimidin-2-yl)-ethylamine (82 mg, 0.34 mmol) in acetonitrile (10 mL) were added DMAP (4 mg, 0.03 mmol), triethylamine (55 μL, 0.40 mmol) and di-tert-butyl dicarbonate (89 mg, 0.41 mmol). The resulting mixture was stirred at RT for 17 h, then EtOAc and water were added. The organic layer was isolated, then washed with brine, dried (MgSO₄) and concentrated in vacuo to give the title compound as a yellow solid (97 mg, 83%).

[M+H —CO₂^{t}Bu]⁺ 243.8

### Reference Example 24

### 4-[6-Chloro-2-(pyridin-3-ylmethoxymethyl)-pyrimidin-4-yl]-morpholine

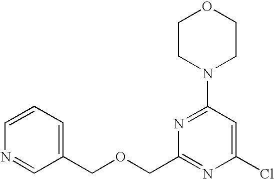

To a solution of 4-(2-bromomethyl-6-chloro-pyrimidin-4-yl)-morpholine (77 mg, 0.70 mmol) and pyridin-3-yl-methanol (205 mg, 0.70 mmol) in DMF (5 ml) at 0° C., under a nitrogen atmosphere, was added sodium hydride (60% dispersion in mineral oil, 26 mg, 0.71 mmol). The reaction mixture was stirred at RT for 18 h. The solvent was removed in vacuo and the resultant residue partitioned between DCM and water. The organic layer was isolated, then washed with brine, dried (Na₂SO₄) and concentrated in vacuo. The title compound was obtained as a white solid (180 mg, 82%).

[M+H]⁺ 321.2, 323.2

### Reference Example 25

### {2-[4-(1H-Indol-4-yl)-6-morpholin-4-yl-pyrimidin-2-yl]-ethyl}-carbamic acid tert-butyl ester

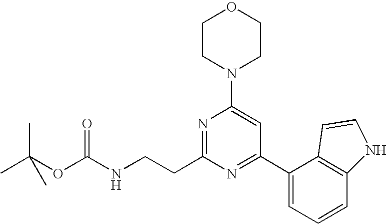

Prepared using method B. The title compound was obtained as a pale yellow oil (35 mg, 31%).

[M+H]⁺ 424.6

### Reference Example 26

### {2-[4-(1-Benzenesulfonyl-1H-indol-4-yl)-6-morpholin-4-yl-pyrimidin-2-yl]-ethyl}-carbamic acid tert-butyl ester

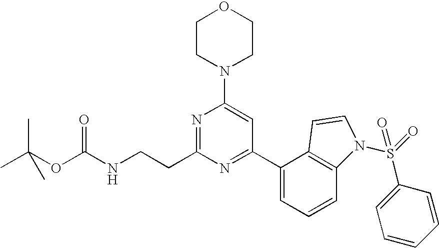

To a solution of {2-[4-(1H-indol-4-yl)-6-morpholin-4-yl-pyrimidin-2-yl]-ethyl}-carbamic acid tert-butyl ester (34 mg, 0.08 mmol) in anhydrous THF (0.5 mL) under an nitrogen atmosphere was added sodium hydride (60% dispersion in mineral oil, 5 mg, 0.13 mmol). The resulting mixture was stirred at RT for 10 min, and a solution of benzenesulfonoyl chloride (15 μL, 0.12 mmol) in anhydrous THF (0.25 mL) was added. The reaction mixture was stirred at RT for 1 h, then an aqueous sat. solution of NaHCO₃ and DCM were added. The organic layer was isolated, then washed with brine, dried (MgSO₄) and concentrated in vacuo. The resultant residue was purified by column chromatography to give the title compound as a colourless oil (43 mg, 95%).

[M+H]⁺ 564.3

### Reference Example 27

### 2-[4-(1-Benzenesulfonyl-4H-indol-4-yl)-6-morpholin-4-yl-pyrimidin-2-yl]-ethylamine

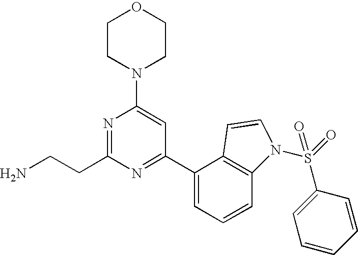

To a solution of {2-[4-(1-benzenesulfonyl-1H-indol-4-yl)-6-morpholin-4-yl-pyrimidin-2-yl]-ethyl}-carbamic acid tert-butyl ester (43 mg, 0.08 mmol) in DCM (3 mL) was added TFA (1 mL) and the resulting mixture stirred at RT for 1 h. The reaction mixture was loaded onto a Isolute® SCX-2 cartridge, washed with MeOH then eluted with 2 M NH₃ in MeOH to give the title compound as a colourless oil (30 mg, 85%).

[M+H]⁺ 464.2

### Reference Example 28

### N-(4-Chloro-6-morpholin-4-yl-pyrimidin-2-ylmethyl)-N-pyridin-3-ylmethyl-methane-sulfonamide

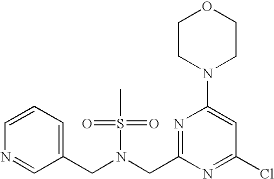

To a solution of N-pyridin-3-ylmethyl-methanesulfonamide (70 mg, 0.37 mmol) in THF (4 mL) at 0° C. was added n-butyl lithium (1.6 M in hexanes, 212 μL, 0.34 mmol) dropwise under a nitrogen atmosphere. The resulting suspension was stirred at 0° C. for 10 min, then a solution of 4-(2-bromomethyl-6-chloro-pyrimidin-4-yl)-morpholine (100 mg, 0.34 mmol) in THF (2 mL) was added in one portion. The resulting suspension was stirred at 0° C. for 30 min, then allowed to warm up to RT and stirred for 18 h. The reaction mixture was partitioned between EtOAc and water and the phases were separated. The organic layer was washed with brine, dried (Na₂SO₄) then concentrated in vacuo. The resultant crude material was purified by column chromatography to give the title compound as an off-white solid (127 mg, 94%).

[M+H]⁺ 398.1

### Reference Example 29

### Pyridine-3-sulfonic acid (4-chloro-6-morpholin-4-yl-pyrimidin-2-ylmethyl)-methyl-amide

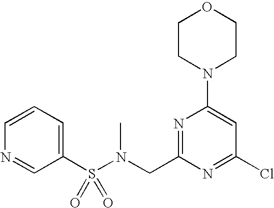

To a solution of pyridine-3-sulfonic acid methylamide (64 mg, 0.37 mmol) in DMF (3 mL) was added sodium hydride (60% in mineral oil, 14 mg, 0.34 mmol) under nitrogen. The solution was stirred at RT for 10 min, then 4-(2-bromomethyl-6-chloro-pyrimidin-4-yl)-morpholine (100 mg, 0.34 mmol) was added in one portion. The solution was stirred at RT for 18 h. The reaction mixture was partitioned between EtOAc and water, and the phases were separated. The organic layer was washed with brine, dried (Na₂SO₄) then concentrated in vacuo. The resultant crude material was purified by column chromatography to give the title compound as a pale yellow solid (107 mg, 82%).

[M+H]⁺ 384.2

### Reference Example 30

### (4-Chloro-6-morpholin-4-yl-pyrimidin-2-ylmethyl)-phenethyl-amine

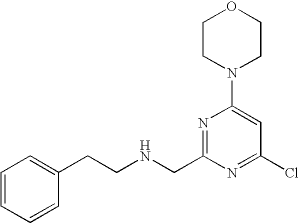

To a solution of 4-(2-bromomethyl-6-chloro-pyrimidin-4-yl)-morpholine (95 mg, 0.33 mmol) in dry acetonitrile (5 mL) was added 2-phenylethylamine (204 mL, 1.63 mmol) and the result and reaction mixture stirred for 45 min. A sat. solution of ammonium chloride was added and the mixture was extracted with EtOAc. The combined organic layers were washed with brine, dried (Na₂SO₄) and concentrated in vacuo. The resultant residue was purified by column chromatography to afford the title compound as a pale brown gum (42 mg, 39%).

[M+H]⁺ 332.8

### Reference Example 31

### N′-(4-Chloro-6-morpholin-4-yl-pyrimidin-2-ylmethyl)-N,N-dimethyl-ethane-1,2-diamine

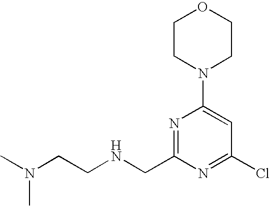

To a solution of 4-(2-bromomethyl-6-chloro-pyrimidin-4-yl)-morpholine (150 mg, 0.51 mmol) in DMF (2 mL) and H₂O (28 mL) was added Cs₂CO₃ (335 mg, 1.03 mmol) and N,N-dimethylethylenediamine (282 mL, 2.57 mmol). The reaction mixture was stirred at RT for 17 h before water and DCM were added. The phases were separated using a hydrophobic frit and the organic phase was concentrated in vacuo. The resultant residue was purified by column chromatography to yield the title compound (47 mg, 31%).

[M+H]⁺ 299.8

### Reference Example 32

### (4-Chloro-6-morpholin-4-yl-pyrimidin-2-ylmethyl)-(2-methoxy-ethyl)-amine

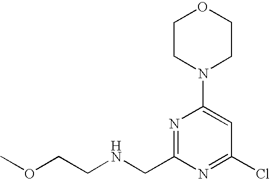

Prepared according to the method used in the preparation of N-(4-chloro-6-morpholin-4-yl-pyrimidin-2-ylmethyl)-N,N-dimethyl-ethane-1,2-diamine using 2-methoxyethylamine (223 mL, 2.57 mmol) in place of N,N-dimethylethylenediamine. The title compound was obtained as a gum (85 mg, 58%).

[M+H]⁺ 286.8

### Reference Example 33

### Benzyl-(4-chloro-6-morpholin-4-yl-pyrimidin-2-ylmethyl)-amine

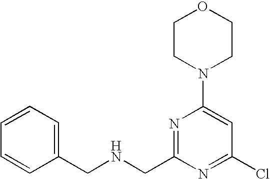

Prepared according to the method used in the preparation of N-(4-chloro-6-morpholin-4-yl-pyrimidin-2-ylmethyl)-N,N-dimethyl-ethane-1,2-diamine using benzylamine (281 mL, 2.57 mmol) in place of N,N-dimethylethylenediamine. The title compound was obtained as an off-white solid (146 mg, 90%).

[M+H]⁺ 318.8

### Reference Example 34

### (4-Chloro-6-morpholin-4-yl-pyrimidin-2-ylmethyl)-[2-(3H-imidazol-4-yl)-ethyl]-amine

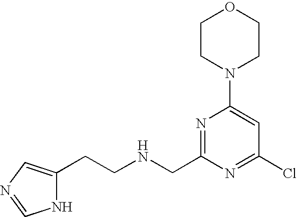

Prepared according to the method used in the preparation of N′-(4-chloro-6-morpholin-4-yl-pyrimidin-2-ylmethyl)-N,N-dimethyl-ethane-1,2-diamine using histamine (285 mg, 2.57 mmol) in place of N,N-dimethylethylenediamine. The title compound was obtained as a gum (40 mg, 24%).

[M+H]⁺ 322.8

### Reference Example 35

### 5-(4-Chloro-6-morpholin-4-yl-pyrimidin-2-ylmethyl)-hexahydro-pyrrolo[3,4-c]pyrrole-2-carboxylic acid tert-butyl ester

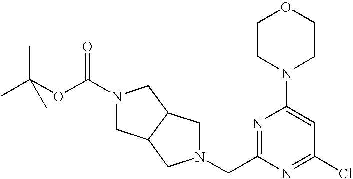

To a solution of 4-(2-bromomethyl-6-chloro-pyrimidin-4-yl)-morpholine (230 mg, 0.786 mmol) in DMF (10 mL) were added potassium carbonate (212 mg, 1.53 mmol) and hexahydro-pyrrolo[3,4-c]pyrrole-2-carboxylic acid tert-butyl ester (250 mg, 1.18 mmol). The mixture was stirred at RT for 30 min. The reaction mixture was diluted with water then extracted into EtOAc. The organic layer was washed with brine, dried (MgSO₄) and concentrated in vacuo. The resultant residue was purified by column chromatography to give the title compound as a colourless oil (332 mg, 100%).

[M+H]⁺ 424.3

### Reference Example 36

### (4-Chloro-6-morpholin-4-yl-pyrimidin-2-ylmethyl)-pyridin-3-ylmethyl-amine

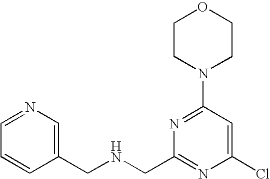

To a suspension of C-(4-chloro-6-morpholin-4-yl-pyrimidin-2-yl)-methylamine (0.15 g, 0.66 mmol) and pyridine-3-carbaldehyde (82 mg, 0.76 mmol) in 1,2-dichloroethane (5 mL) were added glacial acetic acid (0.2 g, 3.33 mmol) and sodium triacetoxyborohydride (0.21 g, 1.00 mmol). The reaction mixture was stirred at RT for 17 h. The solvent was removed by evaporation, and the resulting residue was purified by column chromatography to obtain the title compound as a gum (59 mg, 28%).

[M+H]⁺ 319.8

### Reference Example 37

### N-(4-Chloro-6-morpholin-4-yl-pyrimidin-2-ylmethyl)-nicotinamide

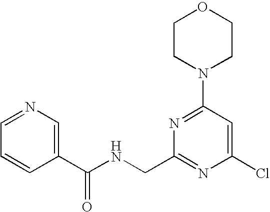

To a solution of C-(4-chloro-6-morpholin-4-yl-pyrimidin-2-yl)-methylamine (0.15 g, 0.66 mmol) and nicotinic acid (90 mg, 0.73 mmol) in DMF (5 mL) were added DIPEA (0.17 g, 1.32 mmol) and HATU (0.25 g, 0.66 mmol). The reaction mixture was stirred at RT for 17 h. The solvent was removed by evaporation and the resultant residue was treated with a sat. solution of NaHCO₃ before being extracted into DCM. The organic layer was isolated then washed with brine, dried (Na₂SO₄) and concentrated in vacuo. The resultant residue was purified by column chromatography to give the title compound as a gum (0.1 g, 45%).

[M+H]⁺ 334.1

### Reference Example 38

### 4-[(4-Chloro-6-morpholin-4-yl-pyrimidin-2-ylmethyl)-carbamoyl]-piperidine-1-carboxylic acid tert-butyl ester

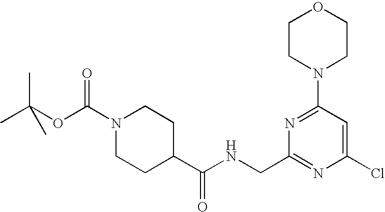

Prepared according to the method used in the preparation of N-(4-chloro-6-morpholin-4-yl-pyrimidin-2-ylmethyl)-nicotinamide using piperidine-1,4-dicarboxylic acid mono-tert-butyl ester in place of nicotinic acid. The title compound was obtained as a colourless gum (80 mg, 34%).

[M+H]⁺ 440.3

### Reference Example 39

### 4-(4-Chloro-6-morpholin-4-yl-pyrimidine-2-carbonyl)-piperazine-1-carboxylic acid tent-butyl ester

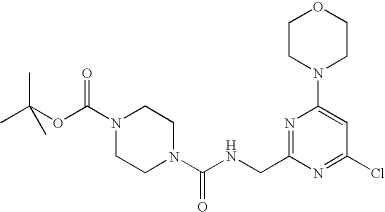

To a solution of piperazine-1-carboxylic acid tert-butyl ester (1.16 mmol) in anhydrous toluene (8 mL) was added trimethylaluminium (2 M in toluene, 0.58 mL, 1.16 mmol) at 0° C. The resulting mixture was stirred at 0° C. for 30 min. 4-Chloro-6-morpholin-4-yl-pyrimidine-2-carboxylic acid methyl ester (300 mg, 1.16 mmol) was added portionwise at 0° C. The reaction mixture was allowed to warm to RT and stirred for 16 h. A solution of NaOH (aq., 4 M) was added drop-wise and the aqueous layer was extracted with EtOAc. The combined organic layers were washed with brine, dried (MgSO₄), then concentrated in vacuo. The resultant residue was purified by column chromatography to give the title compound as a white solid (290 mg, 91%).

[M+H]⁺ 412.2

### Reference Example 40

### 4-[(4-Chloro-6-morpholin-4-yl-pyrimidine-2-carbonyl)-amino]-piperidine-1-carboxylic acid tert-butyl ester

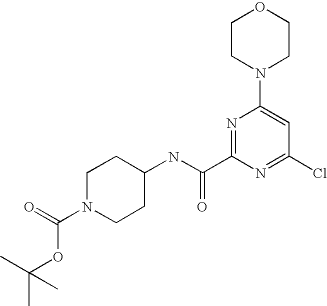

Prepared according to the method used in the preparation 4-(4-Chloro-6-morpholin-4-yl-pyrimidine-2-carbonyl)-piperazine-1-carboxylic acid tert-butyl ester using 4-amino-piperidine-1-carboxylic acid tert-butyl ester in place of piperazine-1-carboxylic acid tert-butyl ester. The title compound was obtained as a white solid (245 mg, 99%).

[M+H]⁺ 426.3

### Reference Example 41

### 4-[4-(4-Chloro-6-morpholin-4-yl-pyrimidin-2-yl)-thiophen-2-ylmethyl]-piperazine-1-carboxylic acid tert-butyl ester

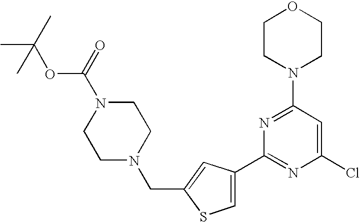

To a solution of 4-(4-chloro-6-morpholin-4-yl-pyrimidin-2-yl)-thiophene-2-carbaldehyde (66 mg, 0.214 mmol) and piperazine-1-carboxylic acid tert-butyl ester (60 mg, 0.321 mmol) in 1,2-dichloroethane (3 mL) was added sodium triacetoxyborohydride (136 mg, 0.643 mmol). The reaction mixture was stirred at RT for 4 h, then DCM and water were added. The aqueous layer was isolated and extracted twice with DCM. The combined organic layers were dried (MgSO₄) and concentrated in vacuo. The resultant residue was purified by column chromatography to obtain the title compound as a tan solid (53 mg, 52%).

[M+H]⁺ 480.1

### Reference Example 42

### 2-(4-Chloro-6-morpholin-4-yl-pyrimidin-2-ylmethyl)-1,2,3,4-tetrahydro-isoquinoline

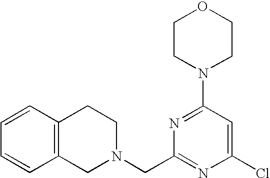

To a solution of 4-(2-bromomethyl-6-chloro-pyrimidin-4-yl)-morpholine (73 mg, 0.26 mmol) and 1,2,3,4-tetrahydroisoquinoline (50 mg, 0.38 mmol) in DMF (2 mL) was added potassium carbonate (69 mg, 0.5 mmol). The mixture was stirred at RT for 3 h. The solvent was concentrated in vacuo and the resultant residue partitioned between DCM and H₂O. The organic layer was washed with brine, dried (Na₂SO₄) and concentrated in vacuo. The resultant residue was crystallised from diethyl ether to obtain the title compound as a white solid (52 mg, 60%).

[M+H]⁺ 345.2

### Reference Example 43

### 1-(4-Chloro-6-morpholin-4-yl-pyrimidin-2-ylmethyl)-1,2,3,4,5,6-hexahydro-[4,4′]bipyridinyl

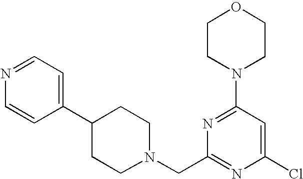

Prepared according to the method used in the preparation of 2-(4-chloro-6-morpholin-4-yl-pyrimidin-2-ylmethyl)-1,2,3,4-tetrahydro-isoquinoline using 1,2,3,4,5,6-hexahydro-[4,4]-bipyridinyl in place of 1,2,3,4-tetrahydroisoquinoline. The title compound was obtained as a colourless gum (200 mg, 100%).

[M+H]⁺ 374.2

### Reference Example 44

### 4-(4-Chloro-6-morpholin-4-yl-pyrimidin-2-yl)-thiophene-2-carbaldehyde

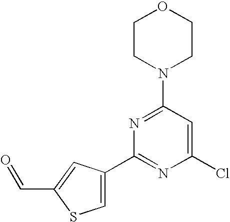

Prepared from 4-(6-chloro-2-iodo-pyrimidin-4-yl)-morpholine and 2-formylthiophene-4-boronic acid using method B (Scheme 1). The title compound was obtained as a pale yellow solid (66 mg, 24%).

[M+H]⁺ 309.8

### Reference Example 45

### 4-[3-(4-Chloro-6-morpholin-4-yl-pyrimidin-2-yl)-phenyl]-piperazine-1-carboxylic acid tert-butyl ester

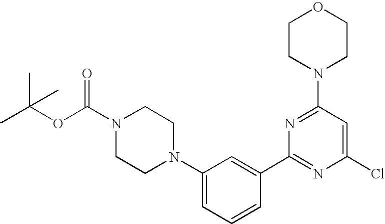

Prepared by method B (Scheme 1) using 4-(6-chloro-2-iodo-pyrimidin-4-yl)-morpholine and 3-(4-tert butoxycarbonyl)piperazine-1-yl)phenyl boronic acid pinacolester. The reaction was carried out for 10 min at 115° C. The title compound was obtained as a light yellow oil (260 mg, 91%).

[M+H]⁺ 460.2

### Reference Example 46

### 4-[6-Chloro-2-(2-pyridin-3-yl-ethyl)-pyrimidin-4-yl]-morpholine

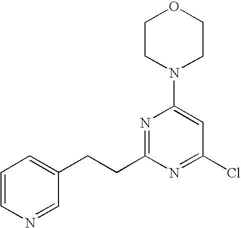

A solution of 4-(2-bromomethyl-6-chloro-pyrimidin-4-yl)-morpholine (129 mg, 0.44 mmol) and triphenylphosphine (173 mg, 0.66 mmol) in toluene (10 mL) was heated to reflux for 2 h. The reaction mixture was allowed to cool to RT and filtered. The precipitate was washed with diethyl ether and dried in vacuo to give (4-chloro-6-morpholin-4-yl-pyrimidin-2-ylmethyl)-triphenyl-phosphonium bromide as a white solid (193 mg, 79%).

[M]⁺ 474.3

A solution of (4-chloro-6-morpholin-4-yl-pyrimidin-2-ylmethyl)-triphenyl-phosphonium bromide (145 mg, 0.261 mmol), 3-formyl-pyridine (25 μL, 0.261 mmol) and triethylamine (36 μL, 0.261 mmol) in toluene (5 mL) was heated to reflux for 24 h. The precipitate was removed by filtration, and the filtrate was concentrated in vacuo. The resultant residue was purified by column chromatography to give 4-[6-chloro-2-(2-pyridin-3-yl-vinyl)-pyrimidin-4-yl]-morpholine in mixture with triphenylphosphine oxide (62 mg). The compound was used without further purification.

[M+H]⁺ 303.0

A suspension of 4-[6-chloro-2-(2-pyridin-3-yl-vinyl)-pyrimidin-4-yl]-morpholine (95 mg, in mixture with triphenylphosphine oxide), palladium on charcoal 5% (20 mg) and acetic acid (2 drops) in ethanol (5 mL) was purged with nitrogen, then stirred under an atmosphere of hydrogen for 2.5 h. The reaction mixture was then purged with nitrogen, filtered over hyflo and washed with ethanol. The filtrate was concentrated in vacuo and the resulting residue purified using a Isolute® SCX-2 cartridge, eluting with MeOH followed by 0.2% NH₃ in MeOH, to give the title compound (40 mg, 77% over 2 steps).

[M+H]⁺ 305.2

### Reference Example 47

### 4-(6-Chloro-2-iodo-pyrimidin-4-yl)-morpholine

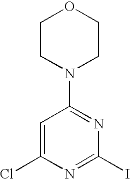

### 4-Chloro-6-morpholin-4-yl-pyrimidin-2-ylamine was prepared as described in Acta Crystallogr. Sect. C: Cryst. Struct. Commun.; EN; 59; 1; 2003; 4-8.

To a mixture of 4-chloro-6-morpholin-4-yl-pyrimidin-2-ylamine (200 mg, 0.93 mmol), diiodomethane (0.37 mL, 4.59 mmol) and copper(I)iodide (177 mg, 0.93 mmol) in tetrahydrofuran (5 mL) was added isoamyl nitrite (0.36 mL, 2.75 mmol). The mixture was flushed out with nitrogen and heated to reflux for 1 hour. The cooled reaction mixture was partitioned between ethyl acetate and 1M hydrochloric acid. The organic layers were washed with concentrated aqueous ammonia followed by saturated aqueous ammonium chloride and dried (MgSO₄). The crude product was purified by column chromatography to give the title compound as a yellow solid (141 mg).

δ_{H} (400 MHz, CDCl₃) 3.63 (br m, 4H), 3.78 (t, J=4.9, 4H), 6.44 (s, 1H).

[M+H]⁺ 325.95

### Reference Example 48

### (2-Chloro-6-morpholin-4-yl-pyrimidin-4-ylmethyl)-dimethyl-amine

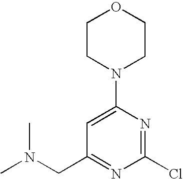

To a solution of 2,6-dichloro-pyrimidine-4-carboxylic acid methyl ester (5.0 g) in anhydrous methanol (40 mL) was added morpholine (4.20 mL). The reaction mixture was stirred at room temperature for 12 hours, then poured onto ice/water and the white precipitate collected by filtration. The solid was washed with water (30 mL) and dried to give 2-chloro-6-morpholin-4-yl-pyrimidine-4-carboxylic acid methyl ester (4.94 g).

To a solution of 2-chloro-6-morpholin-4-yl-pyrimidine-4-carboxylic acid methyl ester (1.0 g) in anhydrous dichloromethane (30 mL) at −78° C. was added diisobutylaluminium hydride (5.82 mL; 1.0 M in dichloromethane). The reaction mixture was stirred at −78° C. for 4 hours, quenched with methanol and allowed to warm to room temperature, then partitioned between water and dichloromethane. The combined organic extracts were dried and purified by flash silica chromatography to give 2-chloro-6-morpholin-4-yl-pyrimidine-4-carbaldehyde as a yellow solid (0.531 g). Reductive amination using the method E gave the title compound as a white solid (0.103 g).

### Reference Example 49

### 4-(2-Methanesulfonyl-6-morpholin-4-yl-pyrimidin-4-yl)-1H-indole

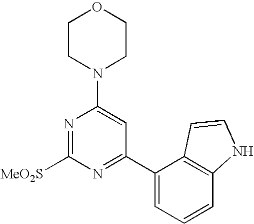

To a stirred solution of 4,6-dichloro-2-(methylthio)pyrimidine (15.44 g; 79 mmol) and DIPEA (15 ml; 86 mmol) in THF (200 mL) at r.t. was added morpholine (7.6 ml; 87 mmol) in one portion (a thick white precipitate forms within a few minutes). The reaction mixture was heated at 60° C. overnight (18 h) during which time all solids dissolve. The cooled reaction mixture was poured into well-stirred water (1.5 L) and the resulting white solid was collected by filtration, washed with water and dried to afford 4-(6-chloro-2-methylsulfanyl-pyrimidin-4-yl)-morpholine as a white solid (19.03 g; 98%).

To a stirred solution of Oxone® (30.74 g; 50 mmol) and Bu₄NHSO₄ (0.68 g; 2.0 mmol) was added a solution of 4-(6-chloro-2-methylsulfanyl-pyrimidin-4-yl)-morpholine (4.91 g; 20 mmol) in CH₂Cl₂ (150 mL). The biphasic mixture was stirred vigorously overnight (18 h) upon which time the remaining CH₂Cl₂ was removed in vacuo. The resulting precipitate was collected by filtration, washed with water and dried to afford 4-(6-chloro-2-methanesulfonyl-pyrimidin-4-yl)-morpholine as a white solid (4.37 g; 79%); [M+H]⁺ 278.

A stirred mixture of 4-(6-chloro-2-methanesulfonyl-pyrimidin-4-yl)-morpholine (1.04 g; 3.74 mmol), indole-4-boronic acid (0.71 g; 4.41 mmol), Pd₂ dba₃ (34 mg; 0.037 mmol), PCy₃ (25 mg; 0.089 mmol), K₃PO₄ (5 mL of a 1.27M aqeuous solution; 6.4 mmol) and dioxane (10 mL) was heated at 125° C. in a microwave for 50 min. The organic layer was separated and the aqueous extracted with a further portion of dioxane (30 ml). The combined organic layers were filtered through a pad of silica (EtOAc as eluent), the solvent was evaporated and the residue triturated with MeOH to afford the title compound as an off-white solid (0.83 g; 62%).

δ_{H} (400 MHz, CDCl₃) 3.41 (s, 3H), 3.82-3.85 (m, 8H), 7.08-7.10 (m, 2H), 7.32, (t, J=8.0, 1H), 7.37 (t, J=2.8, 1H), 7.57 (d, J=8.0), 7.65 (d, J=7.6, 1H), 8.43 (br s, 1H).

[M+H]⁺ 359.

### Reference Example 50

### (4-Chloro-6-morpholin-4-yl-pyrimidin-2-ylmethyl)-methyl-pyridin-3-ylmethyl-amine

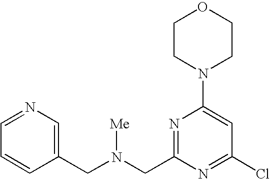

A mixture of 4-(2-bromomethyl-6-chloro-pyrimidin-4-yl)-morpholine (585 mg; 2.0 mmol), N-Methyl-N-(3-pyridylmethyl)amine (367 mg; 3.0 mmol) and Cs₂CO₃ (652 mg; 2.0 mmol) in DMF (10 mL) was stirred at r.t. overnight (18 h). The reaction mixture was diluted with brine (50 ml) and extracted with EtOAc (2×75 mL). The combined organics were dried (Na₂SO₄), concentrated and purified by flash chromatography (90:10:1

CH₂Cl₂/MeOH/NH₄OH as eluent) to afford the title compound as a pale orange oil (585 mg; 88%).

δ_{H} (400 MHz, CDCl₃) 2.39 (s, 3H), 3.64-3.67 (m, 4H), 3.70 (s, 2H), 3.73 (s, 2H), 3.77-3.81 (m, 4H), 6.39 (s, 1H), 7.25-7.28 (m, 1H), 7.77 (d, J=7.6, 1H), 8.50-8.52 (m, 1H), 8.60 (s, 1H).

### Reference Example 51

### (4-Chloro-6-morpholin-4-yl-pyrimidin-2-ylmethyl)-methyl-thiophen-2-ylmethyl-amine

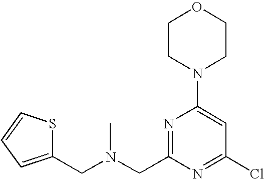

To a solution of thiophene-2-carboxaldehyde (500 mg, 4.46 mmol) in methanol (5 mL) was added methylamine, 2.0 M solution in methanol (5.18 mL, 10.32 mmol) at room temperature. The mixture was stirred overnight. The reaction mixture was evaporated down to give the imine intermediate. This was dissolved in ethanol (8 mL) and platinum(IV)oxide (50 mg) was added. The mixture was purged with nitrogen and stirred under a balloon of hydrogen at room temperature overnight. The mixture was filtered through celite, washed with ethyl acetate and the filtrate evaporated down to give methyl-thiophen-2-ylmethyl-amine (560 mg).

Reaction of this amine with 4-(2-bromomethyl-6-chloro-pyrimidin-4-yl)-morpholine using the method described for (4-chloro-6-morpholin-4-yl-pyrimidin-2-ylmethyl)-methyl-pyridin-3-ylmethyl-amine gave the title compounds as a pale oil (98 mg).

δ_{H} (400 MHz, CDCl₃) 2.43 (s, 3H), 3.67 (m, 4H), 3.73 (s, 2H), 3.80 (t, J=4.8, 4H), 4.14 (s, 2H), 6.39 (s, 1H), 6.97 (m, 2H), 7.25 (m, 2H).

### Reference Example 52

### 4-(4,4,5,5-Tetramethyl-[1,3,2]dioxaborolan-2-yl)-1H-indole-6-carbonitrile

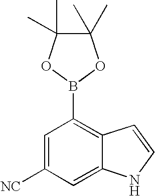

Prepared by using the general method (Scheme 1). The title compound was obtained as an off-white solid.

δH (400 MHz, CDCl₃) 1.40 (s, 12H), 7.12 (m, 1H), 7.46 (t, J=2.9, 1H), 7.8 (t, J=1.1, 1H), 7.87 (d, J=1.3, 1H), 8.42 (br s, 1H).

### Reference Example 53

### 4-(4,4,5,5-Tetramethyl-[1,3,2]dioxaborolan-2-yl)-1H-indole-6-sulfonic acid dimethylamide

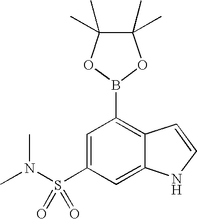

Prepared using the general method of Scheme 1. The title compound was obtained as a white solid (1.85 g, 46%).

[M+H]⁺ 350.2 (¹⁰B) 351.2 (¹¹B)

### Reference Example 54

### 4-(4,4,5,5-Tetramethyl-[1,3,2]dioxaborolan-2-yl)-6-trifluoromethyl-1H-indole

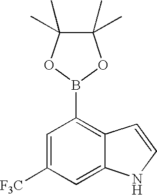

Prepared using the general method of Scheme 1. The title compound was obtained as a pale yellow solid (1.37 g, 92%).

[M+H]⁺ 311.2 (¹⁰B) 312.2 (¹¹B)

### Reference Example 55

### 6-Methanesulfonyl-4-(4,4,5,5-tetramethyl-[1,3,2]dioxaborolan-2-yl)-1H-indole

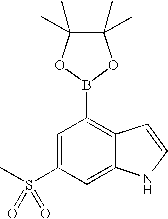

Prepared using the general method of Scheme 1. The title compound was obtained as a pale yellow solid (2.4 g, 51%).

NMR δ_{H} (300 MHz, DMSO-d₆) 1.36 (s, 12H), 3.18 (s, 3H), 6.87 (m, 1H), 7.73 (apparent t, J=2.5, 1H), 7.85 (d, J=1.5, 1H), 8.07 (dd, J=1.0, 1.5, 1H) and 11.73 (bs, 1H).

### Reference Example 56

### 6-Fluoro-4-(4,4,5,5-tetramethyl-[1,3,2]dioxaborolan-2-yl)-1H-indole

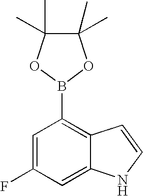

Prepared using the general method of Scheme 1. The title compound was obtained as a white solid (4.6 g, 61%).

NMR δ_{H} (300 MHz, CDCl₃) 1.39 (s, 12H), 7.02 (m, 1H), 7.14-7.19 (m, 1H), 7.20-7.26 (m, 1H), 7.38 (dd, J=2.4, 9.9, 1H) and 8.16 (s, 1H).

### Reference Example 57

### (4-Chloro-6-morpholin-4-yl-pyrimidin-2-ylmethyl)-methyl-quinolin-2-ylmethyl-amine

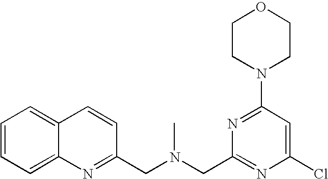

To a stirring solution of 2-quinolinecarboxaldehyde (0.50 g, 3.18 mmol) in methanol (10 mL) was added methylamine, 2.0M solution in methanol (8.0 mL, 15.77 mmol). The reaction mixture was stirred at room temperature overnight. The mixture was evaporated down to give a deep red oil as the imine intermediate (0.58 mg). This was dissolved in methanol (10 mL) and sodium borohydride (0.18 g, 4.76 mmol) was added portionwise. The mixture was stirred at room temperature for 2 hours. The mixture was partitioned between dichloromethane and saturated ammonium chloride. The combined organic layers were washed with brine, separated, dried (MgSO₄) and reduced in vacuo to yield methyl-naphthalen-2-ylmethyl-amine (0.51 g).

δ_{H} (400 MHz, CDCl₃) 2.48 (s, 3H), 4.00 (s, 2H), 7.38 (d, 1H), 7.44 (t, 1H), 7.61 (t, 1H), 7.72 (d, 1H), 7.99 (d, 1H), 8.04 (d, 1H).

The title compound was prepared using the standard alkylation conditions described for (4-chloro-6-morpholin-4-yl-pyrimidin-2-ylmethyl)phenethyl-amine (Reference Example 30) to give a pale yellow oil (160 mg).

δ_{H} (400 MHz, CDCl₃) 2.50 (s, 3H), 3.62 (m, 4H), 3.76 (t, 4H), 4.05 (s, 2H), 4.24 (s, 2H), 6.36 (s, 1H), 7.52 (t, 1H), 7.70 (t, 1H), 7.81 (m, 2H), 8.06 (d, 1H), 8.13 (d, 1H).

### Example 1

### N-[4-(Fluoro-1H-indol-4-yl)-6-morpholin-4-O-pyrimidin-2-ylmethyl]-nicotinamide

Prepared using Method A of Reference Example 2. The title compound was obtained as a white solid (30 mg, 23%)

[M+H]⁺ 433.3

¹H NMR (400 MHz, DMSO-d₆): δ 3.70 (m, 8H), 4.56 (m, 2H), 7.06 (m, 1H), 7.15 (s, 1H), 7.22 (apparent t, J=2.5 Hz, 1H), 7.27 (m, 1H), 7.49-7.56 (m, 2H), 8.27 (m, 1H), 8.73 (dd, J=5, 1.5 Hz, 1H), 9.10 (dd, J=2.5, 1 Hz, 1H), 9.20 (t, J=6 Hz, 1H) and 11.26 (bs, 1H).

### Example 2

### 4-(6-Fluoro-1H-indol-4-yl)-6-morpholin-4-yl-pyrimidin-2-ylmethyl]pyridine-3-ylmethyl-amine

Prepared using Method A of Reference Example 2. The title compound was obtained as a white solid

(12 mg, 16%).

[M+H]⁺ 419.3

¹H NMR (400 MHz, CH₃OH-d₄): δ 3.79 (m, 8H), 4.21 (s, 2H), 4.30 (s, 2H), 6.84 (dd, J=3, 1 Hz, 1H), 7.10 (s, 1H), 7.24 (m, 1H), 7.36 (d, J=3 Hz, 1H), 7.39 (dd, J=10.5, 2.5 Hz, 1H), 7.49 (m, 1H), 7.98 (dt, J=8, 2 Hz, 1H), 8.57 (dd, J=5, 1.5 Hz, 1H) and 8.67 (d, J=2 Hz, 1H).

### Example 3

### Piperidine-4-carboxylic acid [4-(6-fluoro-1H-indol-4-yl)-6-morpholin-4-yl-pyrimidin-2-ylmethyl]-amide

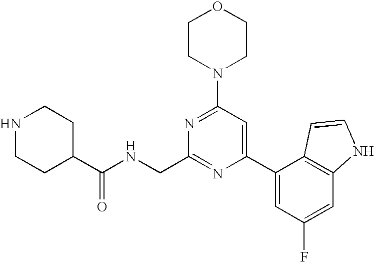

Prepared using Method B of Reference Example 2 followed by BOC-deprotection using TFA:DCM (7:3). The title compound was obtained as a white solid (12 mg, 17%).

[M+H]⁺ 439.3

¹H NMR (400 MHz, CH₃OH-d₄): δ 1.71-1.80 (m, 2H), 1.86-1.95 (m, 2H), 2.48-2.57 (m, 1H), 2.66-2.76 (m, 2H), 3.12-3.19 (m, 2H), 3.73-3.80 (m, 8H), 4.46 (s, 2H), 6.84 (dd, J=3, 1 Hz, 1H), 6.99 (s, 1H), 7.21 (m, 1H) and 7.28-7.34 (m, 2H).

### Example 4

### 4-[2-(Hexahydro-pyrrolo[3,4-c]pyrrol-2-ylmethyl)-6-morpholin-4-yl-pyrimidin-4-yl]-1H-indole

Prepared using Method B of Reference Example 2 followed by BOC-deprotection using TFA:DCM (1:3). The title compound was obtained as a tan solid (102 mg, 32%).

[M+H]⁺ 405.3

¹H NMR (400 MHz, CH₃OH-d₄): δ 2.63 (m, 2H), 2.78-2.95 (m, 8H), 3.72 (s, 2H), 3.76 (m, 4H), 3.80 (m, 4H), 6.80 (dd, J=3, 1 Hz, 1H), 6.98 (s, 1H), 7.21 (apparent t, J=7.5 Hz, 1H), 7.35 (d, J=3 Hz, 1H), 7.44 (dd, J=7.5, 1 Hz, 1H) and 7.50 (dt, J=8, 1 Hz, 1H).

### Example 5

### 4-(1H-Indol-4-yl)-6-morpholin-4-yl-pyrimidine-2-carboxylic acid dimethylamide

To a solution of 4-(1H-indol-4-yl)-6-morpholin-4-yl-pyrimidine-2-carboxylic acid methyl ester (145 mg, 0.429 mmol) in 1,4-dioxane (7 mL) was added an aqueous solution of lithium hydroxide (0.5 M, 3 mL, 1.50 mmol). The resulting suspension was heated at 50° C. for 90 min. The reaction mixture was concentrated by evaporation and the resultant residue dissolved in DMF (7 mL). To this solution were added HATU (203 mg, 0.532 mmol), DIPEA (189 μL, 1.083 mmol) and N,N-dimethylethylenediamine (57 μL, 0.519 mmol). The mixture was stirred at RT for 17 h. Further N,N-dimethylethylenediamine (57 μL, 0.519 mmol) was added, and the mixture was stirred at RT for 24 h. The solvent was removed by evaporation. The resultant residue was purified by preparative HPLC to afford the title compound as a white solid (14.8 mg, 10%).

[M+H]⁺ 352.2

¹H NMR (400 MHz, DMSO-d₆): δ 2.89 (s, 3H), 3.00 (s, 3H), 3.66-3.74 (m, 8H), 6.95 (m, 1H), 7.20 (m, 2H), 7.45 (apparent t, J=3 Hz, 1H), 7.54 (d, J=8 Hz, 1H), 7.62 (dd, J=8, 1 Hz, 1H) and 11.32 (bs, 1H).

### Example 6

### 4-[6-Morpholin-4-yl-2-(pyridin-3-ylmethoxymethyl)-pyrimidin-4-yl]-1H-indole

Prepared using Method C of Reference Example 2. The title compound was obtained as a white solid (54 mg, 43%).

[M+H]⁺ 402.1

¹H NMR (400 MHz, CHCl₃-d): δ 3.68-3.76 (m, 4H), 3.77-3.85 (m, 4H), 4.75 (s, 2H), 4.81 (s, 2H), 6.89 (s, 1H), 7.03 (m, 1H), 7.22-7.31 (m, 3H), 7.46 (d, J=8.5 Hz, 1H), 7.57 (dd, J=7.5, 1 Hz, 1H), 7.82 (m, 1H), 8.47 (bs, 1H), 8.53 (dd, J=5, 1.5 Hz, 1H) and 8.67 (d, J=1.5 Hz, 1H).

### Example 7

### {2-[4-(1H-Indol-4-yl)-6-morpholin-4-yl-pyrimidin-2-yl]-ethyl}-(5-trifluoromethyl-pyridin-2-0)-amine

To a solution of 2-[4-(1-benzenesulfonyl-1H-indol-4-yl)-6-morpholin-4-yl-pyrimidin-2-yl]-ethylamine (30 mg, 0.07 mmol) and NaHCO₃ (6 mg, 0.07 mmol) in acetonitrile (3 mL) was added 2-fluoro-5-(trifluoromethyl)pyridine (12 mg, 0.07 mmol). The resulting mixture was heated at reflux for 21 h, then cooled to RT and concentrated in vacuo. The resulting residue was partitioned between water and DCM and the layers were separated. The organic layer was washed with brine, dried (MgSO₄) and concentrated in vacuo. The resultant residue was purified by column chromatography to give {2-[4-(1-benzenesulfonyl-1H-indol-4-yl)-6-morpholin-4-yl-pyrimidin-2-yl]-ethyl}-(5-trifluoromethyl-pyridin-2-yl)-amine as a colourless oil (30 mg, 76%).

[M+H]⁺ 609.3

To a solution of {2-[4-(1-benzenesulfonyl-1H-indol-4-yl)-6-morpholin-4-yl-pyrimidin-2-yl]-ethyl}-(5-trifluoromethyl-pyridin-2-yl)-amine (30 mg, 0.05 mmol) in IMS (1 mL) and 1,4-dioxane (1 mL) was added an aqueous solution of sodium hydroxide (12 M, 0.1 mL). The resulting mixture was heated at 40° C. for 3 h, then allowed to cool to RT. The pH was adjusted to 8 by careful addition of concentrated HCl and the mixture was concentrated in vacuo. The resulting residue was partitioned between brine and DCM. The organic layer was isolated and loaded onto a Isolute® SCX-2 cartridge, washed with MeOH then eluted with 2 M NH₃ in MeOH to give the title compound as a colourless oil (6.7 mg, 29%).

[M+H]⁺ 469.2

¹H NMR (400 MHz, CH₃OH-d₄): δ 3.10 (t, J=7 Hz, 2H), 3.68-3.73 (m, 4H), 3.76 (m, 4H), 3.88 (t, J=7 Hz, 2H), 6.57 (d, J=9 Hz, 1H), 6.75 (dd, J=2.5, 1 Hz, 1H), 6.92 (s, 1H), 7.20 (apparent t, J=7.5 Hz, 1H), 7.32 (d, J=2.5 Hz, 1H), 7.41 (dd, J=7.5, 1 Hz, 1H), 7.49 (d, J=7.5 Hz, 1H), 7.56 (dd, J=9, 2.5 Hz, 1H) and 8.20 (m, 1H).

### Example 8

### N-[4-(1H-Indol-4-yl)-6-morpholin-4-O-pyrimidin-2-ylmethyl]-N-pyridin-3-ylmethyl-methanesulfonamide

Prepared using Method B of Reference Example 2. The title compound was obtained as a beige solid (33 mg, 22%).

[M+H]⁺ 479.1

1H NMR (400 MHz, DMSO-d₆): δ 3.13 (s, 3H), 3.67 (m, 4H), 3.71 (m, 4H), 4.39 (s, 2H), 4.60 (s, 2H), 6.98 (m, 1H), 7.10 (s, 1H), 7.20 (t, J=8 Hz, 1H), 7.37 (ddd, J=8, 5, 1 Hz, 1H), 7.46 (apparent t, J=3 Hz, 1H), 7.54 (d, J=8 Hz, 1H), 7.61 (dd, J=8, 1 Hz, 1H), 7.78 (dt, J=8, 2 Hz, 1H), 8.49 (dd, J=5, 2 Hz, 1H), 8.53 (d, J=2 Hz, 1H) and 11.31 (bs, 1H).

### Example 9

### Pyridine-3-sulfonic acid [4-(1H-indol-4-yl)-6-morpholin-4-yl-pyrimidin-2-ylmethyl]-methyl-amide

Prepared using Method B of Reference Example 2. The title compound was obtained as a beige solid (93 mg, 72%).

[M+H]⁺ 465.3

¹H NMR (400 MHz, CHCl₃-d): δ 3.11 (s, 3H), 3.56-3.62 (m, 4H), 3.76-3.81 (m, 4H), 4.64 (s, 2H), 6.80 (s, 1H), 6.95 (s, 1H), 7.13-7.18 (m, 1H), 7.23 (m, 1H), 7.31 (s, 1H), 7.42 (d, J=7 Hz, 1H), 7.50 (d, J=8 Hz, 1H), 8.08 (d, J=8 Hz, 1H), 8.34 (s, 1H), 8.57 (m, 1H) and 9.02 (bs, 1H).

### Example 10

### [4-(6-Fluoro-1H-indol-4-yl)-6-morpholin-4-yl-pyrimidin-2-ylmethyl]-phenethyl-amine

Prepared using Method C of Reference Example 2. The title compound was obtained as glass (10 mg, 18%).

[M+H]⁺ 432.3

¹H NMR (400 MHz, CHCl₃-d): δ 2.92 (t, J=7 Hz, 2H), 3.02 (t, J=7 Hz, 2H), 3.62 (t, J=5 Hz, 4H), 3.79 (t, J=5 Hz, 4H), 3.99 (s, 2H), 6.78 (s, 1H), 6.93 (m, 1H), 7.16 (m, 1H), 7.21 (m, 1H), 7.22-7.30 (m, 5H), 7.33 (dd, J=10.5, 2.5 Hz, 1H) and 8.30 (bs, 1H).

### Example 11

### N′-[4-(6-Fluoro-1H-indol-4-yl)-6-morpholin-4-yl-pyrimidin-2-ylmethyl]-N,N-dimethyl-ethane-1,2-diamine

Prepared using Method B of Reference Example 2. The title compound was obtained as an orange gum which solidified upon standing (13 mg, 21%).

[M+H]⁺ 399.1

¹H NMR (400 MHz, CHCl₃-d): δ 2.26 (s, 6H), 2.55 (t, J=6.5 Hz, 2H), 2.87 (t, J=6.5 Hz, 2H), 3.70-3.75 (m, 4H), 3.79-3.85 (m, 4H), 4.00 (s, 2H), 6.82 (s, 1H), 6.96 (m, 1H), 7.17 (dd, J=9, 2 Hz, 1H), 7.28 (dd, J=2.5, 2 Hz, 1H), 7.37 (dd, J=10.5, 2.5 Hz, 1H) and 8.47 (bs, 1H).

### Example 12

### [4-(6-Fluoro-1H-indol-4-yl)-6-morpholin-4-yl-pyrimidin-2-ylmethyl]-(2-methoxy-ethyl)-amine

Prepared using Method C of Reference Example 2. The title compound was obtained as an orange glass (59 mg, 52%).

[M+H]⁺ 386.2

¹H NMR (400 MHz, DMSO-d₆): δ 2.86 (t, J=5.5 Hz, 2H), 3.27 (s, 3H), 3.48 (t, J=5.5 Hz, 2H), 3.71 (m, 8H), 3.85 (s, 2H), 7.03 (m, 1H), 7.13 (s, 1H), 7.30 (m, 1H), 7.45 (dd, J=3, 2.5 Hz, 1H), 7.54 (dd, J=11, 2.5 Hz, 1H) and 11.35 (bs, 1H).

### Example 13

### [4-(6-Fluoro-1H-indol-4-yl)-6-morpholin-4-yl-pyrimidin-2-ylmethyl]-[2-(3H-imidazol-4-yl)-ethyl]-amine

Prepared using Method C of Reference Example 2. The title compound was obtained as a glass. (4.2 mg, 8%).

[M+H]⁺ 422.1

¹H NMR (400 MHz, CH₃OH-d₄): δ 2.98 (t, J=6.5 Hz, 2H), 3.21 (t, J=6.5 Hz, 2H), 3.72 (m, 4H), 3.83 (m, 4H), 4.11 (s, 2H), 6.77 (m, 1H), 6.90 (s, 1H), 6.91 (s, 1H), 7.23 (dd, J=9, 2.5 Hz, 1H), 7.27-7.32 (m, 2H) and 7.53 (s, 1H).

### Example 14

### Benzyl-[4-(6-fluoro-1H-indol-4-yl)-6-morpholin-4-yl-pyrimidin-2-ylmethyl]-amine

Prepared using Method C of Reference Example 2. The title compound was obtained as an orange glass (66 mg, 51%).

[M+H]⁺ 418.1

¹H NMR (400 MHz, CHCl₃-d): δ 3.71 (m, 4H), 3.82 (m, 4H), 3.94 (s, 2H), 4.00 (s, 2H), 6.82 (s, 1H), 6.96 (m, 1H), 7.16 (m, 1H), 7.21-7.30 (m, 2H), 7.30-7.39 (m, 3H), 7.39-7.43 (m, 2H) and 8.35 (bs, 1H).

### Example 15

### 4-(6-Fluoro-1H-indol-4-yl)-6-morpholin-4-yl-pyrimidin-2-yl]-piperazin-1-yl-methanone

Prepared using Suzuki Method A of Reference Example 2 followed by BOC-deprotection using TFA:DCM (1:3). The title compound was obtained as a tan solid (77 mg, 52%).

[M+H]⁺ 411.1

1H NMR (400 MHz, CH₃OH-d₄): δ 2.85 (m, 2H), 2.94 (m, 2H), 3.42 (m, 2H), 3.72-3.81 (m, 10H), 6.87 (dd, J=3.5, 1 Hz, 1H), 7.17 (s, 1H), 7.23 (m, 1H) and 7.32-7.37 (m, 2H).

### Example 16

### 4-(6-Fluoro-1H-indol-4-yl)-6-morpholin-4-yl-pyrimidine-2-carboxylic acid piperidin-4-ylamide

Prepared by using Suzuki Method A followed by BOC-deprotection using TFA:DCM (1:3). The title compound was obtained as a tan solid (93.7 mg, 63%).

[M+H]⁺ 425.3

¹H NMR (400 MHz, CH₃OH-d₄): δ 1.61 (m, 1H), 1.67 (m, 1H), 1.98-2.06 (m, 2H), 2.78 (m, 2H), 3.13 (m, 2H), 3.81 (m, 8H), 4.00-4.09 (m, 1H), 6.89 (dd, J=3.5, 1 Hz, 1H), 7.21-7.27 (m, 2H), 7.37 (d, J=3.5 Hz, 1H) and 7.47 (dd, J=10.5, 2.5 Hz, 1H).

### Example 17

### 6-Fluoro-4-[6-morpholin-4-yl-2-(5-piperazin-1-ylmethyl-thiophen-3-yl)-pyrimidin-4-yl]-1H-indole

Prepared using Method B of Reference Example 2 followed by BOC-deprotection using TFA:DCM (1:1). The title compound was obtained as a white solid (21 mg, 80%).

[M+H]⁺ 479.1

¹H NMR (400 MHz, CHCl₃-d): δ 2.60 (m, 4H), 3.01 (t, J=5 Hz, 4H), 3.75-3.80 (m, 6H), 3.85 (m, 4H), 6.82 (s, 1H), 7.10 (d, J=3 Hz, 1H), 7.16 (m, 1H), 7.28 (d, J=3 Hz, 1H), 7.44 (dd, J=10.5, 2.5 Hz, 1H), 7.75 (m, 1H), 8.22 (d, J=1.5 Hz, 1H) and 8.42 (bs, 1H).

### Example 183

### 6-Fluoro-4-[6-morpholin-4-yl-2-(3-piperazin-1-yl-phenyl)-pyrimidin-4-yl]-1H-indole

Prepared using Method B of Reference Example 2 followed by BOC-deprotection using TFA:DCM (1:3). The title compound was obtained as a beige solid (7.2 mg, 6.5%).

[M+H]⁺ 459.1

¹H NMR (400 MHz, DMSO-d₆): δ 2.87 (m, 4H), 3.13 (m, 4H), 3.76 (m, 4H), 3.80 (m, 4H), 7.08 (dd, J=8.0, 2.5 Hz, 1H), 7.18 (m, 1H), 7.21 (s, 1H), 7.31-7.37 (m, 2H), 7.49 (t, J=2.5 Hz, 1H), 7.62 (dd, J=11.0, 2.5 Hz, 1H), 7.89 (d, J=7.5 Hz, 1H), 8.05 (m, 1H) and 11.37 (bs, 1H).

### Example 19

### 2-[4-(1H-Indol-4-yl)-6-morpholin-4-yl-pyrimidin-2-ylmethyl]-1,2,3,4-tetrahydro-isoquinoline

Prepared using Method B of Reference Example 2. The title compound was obtained as a white solid (55 mg, 44%).

[M+H]⁺ 426.3

¹H NMR (400 MHz, CHCl₃-d): δ 2.99 (t, J=5.5 Hz, 2H), 3.08 (t, J=5.5 Hz, 2H), 3.71 (m, 4H), 3.81 (m, 4H), 3.98 (s, 2H), 4.00 (s, 2H), 6.87 (s, 1H), 7.03 (m, 2H), 7.10 (m, 3H), 7.27 (m, 2H), 7.45 (d, J=8 Hz, 1H), 7.57 (dd, J=7.5, 1 Hz, 1H) and 8.42 (bs, 1H).

### Example 20

### 1-[4-(1H-Indol-4-yl)-6-morpholin-4-yl-pyrimidin-2-ylmethyl]-1,2,3,4,5,6-hexahydro-[4,4′]bipyridinyl

Prepared using Method B of Reference Example 2. The title compound was obtained as a white solid (60 mg, 50%).

[M+H]⁺ 455.1

¹H NMR (400 MHz, CH₃OH-d₄): δ 1.86-1.93 (m, 4H), 2.42-2.52 (m, 2H), 2.66 (m, 1H), 3.33 (m, 2H), 3.77-3.82 (m, 10H), 6.84 (dd, J=3, 1 Hz, 1H), 7.01 (s, 1H), 7.22 (apparent t, J=7.5 Hz, 1H), 7.34 (m, 3H), 7.48 (dd, J=7.5, 1 Hz, 1H), 7.51 (dt, J=8, 1 Hz, 1H), 8.42 (d, J=1.5 Hz, 1H) and 8.43 (d, J=1.5 Hz, 1H).

### Example 21

### 4-[6-Morpholin-4-yl-2-(2-pyridin-3-yl-ethyl)-pyrimidin-4-yl]-1H-indole

Prepared using Method B of Reference Example 2. The title compound was obtained as a white foam (35 mg, 70%).

[M+H]⁺ 386.1

¹H NMR (400 MHz, CH₃OH-d₄): δ 3.15 (t, J=7 Hz, 2H), 3.23 (t, J=7 Hz, 2H), 3.70 (m, 4H), 3.77 (m, 4H), 6.67 (m, 1H), 6.90 (s, 1H), 7.20 (apparent t, J=7.5 Hz, 1H), 7.31-7.39 (m, 3H), 7.49 (d, J=8 Hz, 1H), 7.76 (m, 1H), 8.34 (dd, J=5, 1.5 Hz, 1H) and 8.42 (d, J=2 Hz, 1H).

### Example 22

### 4-[4-(4-Methyl-piperazin-1-yl)-6-morpholin-4-yl-pyrimidin-2-yl]-1H-indole

4-(4-Chloro-6-morpholin-4-yl-pyrimidin-2-yl)-1H-indole was prepared from 4-(6-chloro-2-iodo-pyrimidin-4-yl)-morpholine using method C of Reference Example 2 to give an off-white solid (1.17 g). 4-(4-Chloro-6-morpholin-4-yl-pyrimidin-2-yl)-1H-indole (100 mg, 0.31 mmol), 1-methylpiperazine (53 μl, 0.47 mmol) and 1-methyl-2-pyrrolidinone (2 ml) were sealed in a tube and heated to 150° C. overnight. The mixture was partitioned between ethyl acetate and brine, separated, and dried (MgSO₄). The crude product was purified by column chromatography to yield the title compound (93 mg).

δ_{H} (400 MHz, CDCl₃) 2.39 (s, 3H), 2.57 (t, J=5.0, 4H), 3.70 (t, J=4.8, 4H), 3.70 (t, J=5.0, 4H), 3.86 (t, J=4.8, 4H), 5.61 (s, 1H), 7.23-7.33 (m, 2H), 7.45-7.50 (m, 2H), 8.18 (d, J=5.4, 1H), 8.23 (br s, 1H).

[M+H]⁺ 379.19

### Example 23

### [2-(1H-Indol-4-yl)-6-morpholin-4-yl-pyrimidin-4-yl]-methyl-phenethyl-amine

4-(4-Chloro-6-morpholin-4-yl-pyrimidin-2-yl)-1H-indole was prepared from 4-(6-chloro-2-iodo-pyrimidin-4-yl)-morpholine using Method A of Reference Example 2.

Reaction with N-methyl-2-phenethylamine using the method described for 4-[4-(4-methyl-piperazin-1-yl)-6-morpholin-4-yl-pyrimidin-2-yl]-1H-indole gave an off-white foam (109 mg).

δ_{H} (400 MHz, CDCl₃) 3.01 (t, J=7.5, 2H), 3.08 (s, 3H), 3.70 (t, J=4.9, 4H), 3.87 (t, J=4.9, 4H), 3.94 (t, J=7.5, 2H), 5.44 (s, 1H), 7.22-7.34 (m, 7H), 7.49 (d, J=8.0, 1H), 7.57 (m, 1H), 8.22 (br s, 1H), 8.27 (d, J=8.4, 1H).

[M+H]⁺ 414.18

### Example 24

### 2-(1H-Indol-4-yl)-6-morpholin-4-yl-pyrimidin-4-ylmethyl]-phenethyl-amine

(2-Chloro-6-morpholin-4-yl-pyrimidin-4-ylmethyl)-phenethyl-amine was prepared from 2-chloro-6-morpholin-4-yl-pyrimidine-4-carbaldehyde and phenethylamine using method F of Reference Example 2. Method C of Reference Example 2 then gave the title compound as a white solid (55 mg)

δ_{H} (400 MHz, CDCl₃) 2.56 (t, J=6.7, 2H), 2.65 (t, J=7.0, 2H), 3.37 (t, J=4.8, 4H), 3.48 (t, J=5.3, 4H), 3.54 (s, 2H), 6.07 (s, 1H), 6.86-6.96 (m, 6H), 7.07 (m, 1H), 7.14 (d, J=8.0, 1H), 7.77 (d, J=7.5, 1H), 7.90 (s, br, 1H).

[M+H]⁺ 414.19

### Example 25

### [2-(1H-Indol-4-yl)-6-morpholin-4-yl-pyrimidin-4-ylmethyl]-dimethyl-amine

Suzuki reaction of (2-chloro-6-morpholin-4-yl-pyrimidin-4-ylmethyl)-dimethyl-amine (0.103 g) with 4-indole-boronic acid (0.116 g) using Method A of Reference Example 2 gave the title compound as an off-white solid (0.070 g).

δ_{H} (400 MHz, CDCl₃) 2.42 (s, 6H), 3.63 (s, 2H), 3.85 (m, 4H), 3.87 (m, 4H), 6.69 (s, 1H), 7.30 (m, 2H), 7.52 (m, 2H), 8.19 (d, 1H), 8.21 (br s, 1H).

[M+H]⁺ 338.2.

### Example 26

### Benzyl-[2-(1H-indol-4-yl)-6-morpholin-4-yl-pyrimidin-4-ylmethyl]-methyl-amine

Reaction of 2-chloro-6-morpholin-4-yl-pyrimidine-4-carbaldehyde (0.15 g) with N-benzyl-methylamine (0.165 g) using Method E of Reference Example 2 gave benzyl-(2-chloro-6-morpholin-4-yl-pyrimidin-4-ylmethyl)-methyl-amine, as a white solid (0.101 g). Suzuki reaction, of benzyl-(2-chloro-6-morpholin-4-yl-pyrimidin-4-ylmethyl)-methyl-amine (0.10 g), with 4-indole-boronic acid (0.087 g) using Method A of Reference Example 2, gave the title compound as an off-white solid (16 mg).

δ_{H} (400 MHz, CDCl₃) 2.38 (s, 3H), 3.69 (s, 2H), 3.73 (s, 2H), 3.80 (m, 4H), 3.87 (m, 4H), 6.78 (s, 1H), 7.55-7.30 (m, 8H), 7.70 (m, 1H), 8.18 (d, 1H), 8.19 (br s, 1H).

[M+H]⁺ 414.2.

### Example 27

### Benzyl-[2-(1H-indol-4-yl)-6-morpholin-4-yl-pyrimidin-4-ylmethyl]-amine

Reaction of 2-chloro-6-morpholin-4-yl-pyrimidine-4-carbaldehyde (0.155 g), with benzylamine (0.080 g) using Method A of Reference Example 2 gave benzyl-(2-chloro-6-morpholin-4-yl-pyrimidin-4-ylmethyl)-amine, as a white solid (0.195 g). Suzuki reaction of benzyl-(2-chloro-6-morpholin-4-yl-pyrimidin-4-ylmethyl)-amine (0.11 g), with 4-indole-boronic acid (0.10 g), using Method A, gave the title compound as an off-white solid (0.094 g).

δ_{H} (400 MHz, CDCl₃) 3.69 (m, 4H), 3.77 (m, 4H), 3.82 (s, 2H), 3.84 (s, 2H), 6.42 (s, 1H), 7.33-7.19 (m, 7H), 7.40 (m, 2H), 8.12 (d, J=7.4, 1H), 8.19 (br s, 1H).

[M+H]⁺ 400.2.

### Example 28

### [2-(1H-Indol-4-yl)-6-morpholin-4-yl-pyrimidin-4-ylmethyl]-methyl-pyridin-3-ylmethyl-amine

Reaction of 2-chloro-6-morpholin-4-yl-pyrimidine-4-carbaldehyde (0.15 g), with N-methyl-N-(3-pyridylmethyl)amine (0.161 g) using Method E of Reference Example 2 gave (2-chloro-6-morpholin-4-yl-pyrimidin-4-ylmethyl)-methyl-pyridin-3-ylmethyl-amine, as a white solid (0.207 g).

Suzuki reaction of (2-chloro-6-morpholin-4-yl-pyrimidin-4-ylmethyl)-methyl-pyridin-3-ylmethyl-amine (0.10 g), with 4-indole-boronic acid (0.087 g) using Method A of Reference Example 2 gave the title compound as a white solid (0.075 g).

δ_{H} (400 MHz, CDCl₃) 2.38 (s, 3H), 3.71 (s, 2H), 3.73 (s, 2H), 3.81 (m, 4H), 3.88 (m, 4H), 6.73 (s, 1H), 7.30 (m, 3H), 7.52 (m, 2H), 7.74 (d, J=7.8, 1H), 8.19 (d, J=6.9, 1H), 8.30 (br s, 1H), 8.68 (s, 1H).

[M+H]⁺ 415.2.

### Example 29

### [2-(1H-Indol-4-yl)-6-morpholin-4-yl-pyrimidin-4-ylmethyl]-pyridin-3-ylmethyl-amine

Reaction of 2-chloro-6-morpholin-4-yl-pyrimidine-4-carbaldehyde (0.15 g), with 3-(aminomethyl)pyridine (0.134 g) using Method E of Reference Example 2 gave (2-chloro-6-morpholin-4-yl-pyrimidin-4-ylmethyl)-pyridin-3-ylmethyl-amine, as a white solid (0.14 g). Suzuki reaction of (2-chloro-6-morpholin-4-yl-pyrimidin-4ylmethyl)-pyridin-3-ylmethyl-amine (0.14 g), with 4-indole-boronic acid (0.127 g), using Method A of Reference Example 2, gave the title compound as an off-white solid (0.083 g).

δ_{H} (400 MHz, CDCl₃) 3.80 (m, 4H), 3.87 (m, 4H), 3.89 (s, 2H), 3.94 (s, 2H), 6.48 (s, 1H), 7.31 (m, 2H), 7.48 (m, 2H), 7.76 (m, 1H), 8.22 (d, J=8.4, 1H), 8.30 (br s, 1H), 8.60 (d, J=6.3, 1H), 8.66 (s, 1H).

[M+H]⁺ 401.2.

### Example 30

### [2-(1H-Indol-4-yl)-6-morpholin-4-yl-pyrimidin-4-ylmethyl]-(2-methoxy-ethyl)-amine

Reaction of 2-chloro-6-morpholin-4-yl-pyrimidine-4-carbaldehyde (0.15 g), with 2-methoxyethylamine (0.070 g) using Method A of Reference Example 2 gave (2-chloro-6-morpholin-4-yl-pyrimidin-4-ylmethyl)-(2-methoxy-ethyl)-amine, as a colourless oil (0.142 g). Suzuki reaction of (2-chloro-6-morpholin-4-yl-pyrimidin-4-ylmethyl)-(2-methoxy-ethyl)-amine (0.14 g) with 4-indole-boronic acid (0.144 g) using Method A of Reference Example 2 gave the title compound as a yellow oil (0.095 g).

δ_{H} (400 MHz, CDCl₃) 2.94 (m, 2H), 3.41 (s, 3H), 3.60 (m, 2H), 3.79 (m, 4H), 3.87 (m, 4H), 3.94 (s, 2H), 6.56 (s, 1H), 7.34 (m, 2H), 7.51 (m, 2H), 8.20 (d, J=7.4, 1H), 8.29 (br s, 1H).

[M+H]⁺ 368.2

### Example 31

### [2-(1H-imidazol-4-yl)-ethyl]-[2-(1H-indol-4-yl)-6-morpholin-4-yl-pyrimidin-4-ylmethyl]-amine

Reaction of 2-chloro-6-morpholin-4-yl-pyrimidine-4-carbaldehyde (150 mg) and histamine (81 mg) using Method F of Reference Example 2 gave (2-chloro-6-morpholin-4-yl-pyrimidin-4-ylmethyl)₄₂-(1H-imidazol-4-yl)-ethyl]-amine as a white solid (0.106 g). Suzuki reaction of (2-chloro-6-morpholin-4-yl-pyrimidin-4-ylmethyl)-[2-(1H-imidazol-4-yl)-ethyl]-amine (0.104 g), with 4-indole-boronic acid (0.095 g), using Method A of Reference Example 2, gave the title compound as a white solid (0.014 g).

δ_{H} (400 MHz, DMSO) 3.20 (s, 2H), 3.77 (m, 12H), 5.55 (s, 1H), 7.04 (s, 1H), 7.20 (m, 2H), 7.46 (m, 1H), 7.56 (d, J=8.0, 1H), 7.70 (s, 1H), 8.16 (d, J=7.8, 1H), 11.3 (br s, 1H).

[M+H]⁺ 404.4

### Example 32

### [2-(1H-Indol-4-yl)-6-morpholin-4-yl-pyrimidin-4-ylmethyl]-(1-phenyl-ethyl)-amine

Reaction of 2-chloro-6-morpholin-4-yl-pyrimidine-4-carbaldehyde (0.15 g), with α-methylbenzylamine (0.090 g) using Method E of Reference Example 2 gave (2-chloro-6-morpholin-4-yl-pyrimidin-4-ylmethyl)-(1-phenyl-ethyl)-amine as a colourless oil (0.214 g). Suzuki reaction of (2-chloro-6-morpholin-4-yl-pyrimidin-4-ylmethyl)-(1-phenyl-ethyl)-amine (0.210 g) with 4-indole-boronic acid (0.195 g) using Method A of Reference Example 2 gave the title compound as an off-white solid (0.178 g).

δ_{H} (400 MHz, CDCl₃) 3.69 (s, 2H), 3.75 (m, 4H), 3.83 (m, 4H), 3.91 (m, 1H), 6.39 (s, 1H), 7.54-7.26 (m, 9H), 8.19 (d, J=8.3, 1H), 8.28 (br s, 1H).

[M+H]⁺ 414.2

### Example 33

### [2-(1H-Indol-4-yl)-6-morpholin-4-yl-pyrimidin-4-ylmethyl]-(2-morpholin-4-yl-ethyl)-amine

Reaction of 2-chloro-6-morpholin-4-yl-pyrimidine-4-carbaldehyde (0.15 g) and 4-(2-aminoethyl) morpholine (0.094 g) using Method F of Reference Example 2 gave (2-chloro-6-morpholin-4-yl-pyrimidin-4-ylmethyl)-(2-morpholin-4-yl-ethyl)-amine, as a colourless oil (0.222 g). Suzuki reaction of (2-chloro-6-morpholin-4-yl-pyrimidin-4-ylmethyl)-(2-morpholin-4-yl-ethyl)-amine (0.22 g) with 4-indole-boronic acid (0.188 g), using Method A of Reference Example 2, gave the title compound as an off-white solid (0.066 g).

δ_{H} (400 MHz, CDCl₃) 2.45 (m, 4H), 2.61 (t, J=11.9, 2H), 2.85 (t, J=11.9, 2H), 3.64 (m, 4H), 3.79 (m, 4H), 3.86 (m, 4H), 3.94 (s, 2H), 6.49 (s, 1H), 7.31 (m, 2H), 7.49 (m, 2H), 8.18 (d, J=8.4, 1H), 8.34 (br s, 1H).

[M+H]⁺ 424.3.

### Example 34

### [2-(6-Fluoro-1H-indol-4-yl)-6-morpholin-4-yl-pyrimidin-4-ylmethyl]-methyl-pyridin-3-ylmethyl-amine

(2-chloro-6-morpholin-4-yl-pyrimidin-4-ylmethyl)-methyl-pyridin-3-ylmethyl-amine (0.127 g), prepared using Method E of Reference Example 2, was reacted with 6-fluoro-4-(4,4,5,5-tetramethyl-[1,3,2]dioxaborolan-2-yl)-1H-indole (0.179 g), using Method A of Reference Example 2, to give the title compound as a white solid (0.042 g).

δ^{H} (400 MHz, CDCl₃) 2.38 (s, 3H), 3.70 (s, 2H), 3.72 (s, 2H), 3.80 (m, 4H), 3.88 (m, 4H), 6.74 (s, 1H), 7.18 (d, J=8.8, 1H), 7.31 (m, 2H), 7.51 (s, 1H), 7.73 (d, J=7.8, 1H), 7.97 (d, J=11.3, 1H), 8.29 (br s, 1H), 8.54 (d, J=4.8, 1H), 8.68 (s, 1H).

[M+H]⁺ 433.2

### Example 35

### [2-(6-Methanesulfonyl-1H-indol-4-yl)-6-morpholin-4-yl-pyrimidin-4-ylmethyl]-methyl-pyridin-3-ylmethyl-amine

Prepared using the method described for [2-(6-fluoro-1H-indol-4-yl)-6-morpholin-4-yl-pyrimidin-4-ylmethyl]-methyl-pyridin-3-ylmethyl-amine. White solid (43 mg).

δ_{H} (400 MHz, CDCl₃) 2.39 (s, 3H), 3.15 (s, 3H), 3.72 (s, 2H), 3.73 (s, 2H), 3.80-3.83 (m, 4H), 3.87-3.90 (m, 4H), 6.78 (s, 1H), 7.28-7.32 (m, 1H), 7.57 (t, J=2.6, 1H), 7.64 (s, 1H), 7.75 (d, J=7.6, 1H), 8.15 (s, 1H), 8.55 (d, J=4.8, 1H), 8.69 (s, 1H), 8.73 (br s, 2H).

[M+H]⁺ 493.

### Example 36

### [2-(5-Fluoro-1H-indol-4-yl)-6-morpholin-4-yl-pyrimidin-4-ylmethyl]-methyl-pyridin-3-ylmethyl-amine

Prepared using the method described for [2-(6-fluoro-1H-indol-4-yl)-6-morpholin-4-yl-pyrimidin-4-ylmethyl]-methyl-pyridin-3-ylmethyl-amine. White solid (22 mg).

δ_{H} (400 MHz, CDCl₃) 2.38 (s, 3H), 3.71 (s, 4H), 3.75-3.77 (m, 4H), 3.83-3.85 (m, 4H), 6.77 (s, 1H), 6.90-6.91 (m, 1H), 7.04 (dd, J=10.8 and 8.8, 1H), 7.28-7.30 (m, 2H), 7.37 (dd, J=8.8 and 4.8, 1H), 7.73 (d, J=8.0, 1H), 8.24 (br s, 1H), 8.54 (dd, J=4.8 and 1.2, 1H), 8.68 (s, 1H).

[M+H]⁺ 433.

### Example 37

### [4-(1H-Indol-4-yl)-6-morpholin-4-yl-pyrimidin-2-ylmethyl]-methyl-thiophen-2-ylmethyl-amine

Prepared from (4-chloro-6-morpholin-4-yl-pyrimidin-2-ylmethyl)-methyl-thiophen-2-ylmethyl-amine using Method A of Reference Example 2 to give an off-white solid (54 mg).

δ_{H} (400 MHz, CDCl₃) 2.52 (s, 3H), 3.76 (t, J=4.6, 4H), 3.85 (t, J=4.7, 4H), 3.92 (s, 2H), 4.08 (s, 2H), 6.89 (s, 1H), 6.95-7.00 (m, 2H), 7.08 (s, 1H), 7.26 (m, 3H), 7.49 (d, J=8.1, 1H), 7.61 (d, J=8.1, 1H), 8.32 (br s, 1H).

[M+H]⁺ 420.10

### Example 38

### (1-Benzyl-piperidin-4-yl)-[4-(1H-indol-4-yl)-6-morpholin-4-yl-pyrimidin-2-yl]-amine

To a stirred solution of 2,4,6-trichloropyrimidine (2.0 ml; 17.4 mmol) and DIPEA (3.2 ml; 18.4 mmol), in MeOH (50 ml) at 0° C. was added 4-amino-1-benzylpiperidine (3.7 ml; 18.1 mmol) and the resulting solution was stirred at 0° C. for 1 h and then r.t. overnight (18 h). The reaction mixture was evaporated onto silica and purified by flash chromatography (100:0 to 90:10 EtOAc/MeOH as eluent) to obtain the two regioisomeric products: (1-benzyl-piperidin-4-yl)-(4,6-dichloro-pyrimidin-2-yl)-amine as a white solid (1.29 g; 22%); (1-benzyl-piperidin-4-yl)-(2,6-dichloro-pyrimidin-4-yl)-amine (2.34 g; 40%).

To a stirred solution of 1-benzyl-piperidin-4-yl)-(4,6-dichloro-pyrimidin-2-yl)-amine (169 mg; 0.5 mmol) and DIPEA (0.1 ml; 0.6 mmol) in dioxane (5 ml) and THF (3 ml) at 0° C. was added morpholine (0.1 ml; 1.1 mmol) and the resulting solution was stirred at r.t. overnight (16 h) and then 90° C. for 8 h. The reaction mixture was diluted with brine (30 ml) and extracted with EtOAc (50 ml). The organic layer was dried (Na₂SO₄), concentrated and purified by flash chromatography (98:2:1 EtOAc/MeOH/NEt₃ as eluent) to afford (1-benzyl-piperidin-4-yl)-(2-chloro-6-morpholin-4-yl-pyrimidin-4-yl)-amine as an off-white foam (142 mg; 73%).

A stirred mixture of (1-benzyl-piperidin-4-yl)-(2-chloro-6-morpholin-4-yl-pyrimidin-4-yl)-amine (78 mg; 0.20 mmol), indole-4-boronic acid (40 mg; 0.25 mmol), Cs₂CO₃ (130 mg; 0.40 mmol), Pd(PPh₃)₄ (2.3 mg; 0.002 mmol) and dioxane/H₂O (1:1; 2 ml) was heated in a microwave at 125° C. for 30 min. A further portion of Pd(PPh₃)₄ (9.2 mg; 0.008 mmol) was added and the mixture was heated in the microwave at 125° C. for a further 30 min. The organic layer was separated and purified directly by flash chromatography (98:2:1 EtOAc/MeOH/NEt₃ as eluent) to afford the title compound as a buff-coloured solid (71 mg).

δ_{H} (400 MHz, CDCl₃) 1.54-1.66 (m, 2H), 2.07-2.28 (m, 4H), 2.85-2.89 (m, 2H), 3.56 (s, 2H), 3.65 (t, J=4.8, 4H), 3.76 (t, J=4.8, 4H), 3.97 (br s, 1H), 4.89 (br s, 1H), 6.39 (s, 1H), 7.04 (br s, 1H), 7.26-7.39 (m, 6H), 7.46 (d, J=8.4, 1H), 7.54 (d, J=7.2, 1H), 8.30 (br s, 1H).

[M+H]⁺ 469.

### Example 39

### 4-[4-(4-Methyl-piperazin-1-yl)-6-morpholin-4-yl-pyrimidin-2-yl]-1H-indole

A stirred solution of 4-(2-methanesulfonyl-6-morpholin-4-yl-pyrimidin-4-yl)-1H-indole (30 mg; 0.084 mmol) and N-methylpiperazine (0.05 ml; 0.45 mmol) in NMP (0.5 ml) was heated at 150° C. for 24 h. The reaction mixture was purified directly by preparative LCMS to afford the title compound as a buff-coloured solid (8 mg).

δ_{H} (400 MHz, CDCl₃) 2.40 (br s, 3H), 2.55 (br s, 4H), 3.67 (t, J=4.8, 4H), 3.83 (t, J=4.8, 4H), 3.96 (br s, 4H), 6.40 (s, 1H), 7.09 (s, 1H), 7.28-7.32 (m, 2H), 7.47 (d, J=8.0, 1H), 7.58 (d, J=7.2, 1H), 8.27 (br s, 1H).

[M+H]⁺ 379.

### Example 40

### 1-[2-(1H-Indol-4-yl)-6-morpholin-4-yl-pyrimidin-4-yl]-4-phenyl-piperidin-4-ol

Prepared from 4-hydroxy-4-phenylpiperidine using general Method G of Reference Example 2: off-white solid (87 mg).

δ_{H} (400MHz, CDCl₃) 1.87 (m, 2H), 2.19 (dt, J=13.0 and 4.8, 2H), 3.50 (dt, J=13.0 and 2.4, 2H), 3.69 (t, J=4.8, 4H), 3.73 (s, 1H), 3.84 (t, J=4.8, 4H), 4.87 (m, 2H), 6.40 (s, 1H), 7.12 (m, 1H), 7.25-7.31 (m, 3H), 7.37-7.41 (m, 2H), 7.47 (d, J=8.0, 1H), 7.54-7.58 (m, 2H), 7.61 (d, J=8.0, 1H), 8.26 (br s, 1H).

[M+H]⁺ 401.

### Example 41

### 1-[2-(1H-Indol-4-yl)-6-morpholin-4-yl-pyrimidin-4-0′-piperidine-4-carboxylic acid ethyl ester

Prepared from ethyl isonipecotate using Method G of Reference Example 2: white solid (63 mg).

δ_{H} (400 MHz, CDCl₃) 1.29 (t, J=7.2, 3H), 1.76-1.84 (m, 2H), 1.98-2.03 (m, 2H), 2.55-2.60 (m, 1H), 3.05-3.12 (m, 2H), 3.67 (t, J=4.8, 4H), 3.83 (t, J=4.8, 4H), 4.18 (q, J=7.2, 2H), 4.80-4.85 (m, 2H), 6.38 (s, 1H), 7.08-7.10 (m, 1H), 7.28-7.33 (m, 2H), 7.46-7.48 (m, 1H), 7.57-7.59 (m, 1H), 8.25 (br s, 1H).

[M+H]⁺ 436.

### Example 42

### 1-[2-(1H-Indol-4-yl)-6-morpholin-4-yl-pyrimidin-4-yl]-4-phenyl-piperidine-4-carbonitrile

Prepared from 4-cyano-4-phenylpiperidine HCl using Method G: cream-coloured solid (63 mg).

δ_{H} (400 MHz, CDCl₃) 2.07-2.23 (m, 4H), 3.37-3.45 (m, 2H), 3.69 (t, J=4.8, 4H), 3.84 (t, J=4.8, 4H), 5.12-5.16 (m, 2H), 6.43 (s, 1H), 7.09-7.10 (m, 1H), 7.26-7.61 (m, 9H), 8.28 (br s, 1H).

[M+H]⁺ 465.

### Example 43

### [2-(1H-Indol-4-yl)-6-morpholin-4-yl-pyrimidin-4-yl]-(2-phenoxy-ethyl)-amine

Prepared from 2-phenoxyethylamine using Method G of Reference Example 2: off-white solid (72 mg).

δ_{H} (400 MHz, CDCl₃) 3.64 (t, J=4.8, 4H), 3.79 (t, J=4.8, 4H), 3.91 (q, J=5.6, 2H), 4.17 (t, J=5.6, 2H), 5.36 (br s, 1H), 6.40 (s, 1H), 6.92-6.36 (m, 3H), 7.02 (s, 1H), 7.23-7.29 (m, 4H), 7.44 (d, J=8.0, 1H), 7.53 (d, J=7.2, 1H), 8.32 (br s, 1H).

[M+H]⁺ 416.

### Example 44

### Methyl-[4-morpholin-4-yl-6-(6-trifluoromethyl-1H-indol-4-yl)-pyrimidin-2-ylmethyl]-pyridin-3-ylmethyl-amine

Prepared from (4-chloro-6-morpholin-4-yl-pyrimidin-2-ylmethyl-methyl-pyridin-3-ylmethyl-amine using Method D of Reference Example 2. Title compound obtained as a white solid (14 mg).

δ_{H} (400 MHz, CDCl₃) 2.48 (s, 3H), 3.77 (t, J=4.8, 4H), 3.84-3.89 (m, 8H), 6.87 (s, 1H), 7.14 (br s, 1H), 7.25-7.28 (m, 1H), 7.45-7.47 (m, 1H), 7.78 (s, 1H), 7.82 (s, 1H), 7.86 (d, J=7.6, 1H), 8.52-8.53 (m, 1H), 8.59 (br s, 1H), 8.65 (s, 1H).

[M+H]⁺ 483.

### Example 45

### [4-(6-Fluoro-1H-indol-4-yl)-6-morpholin-4-O-pyrimidin-2-ylmethyl]-methyl-pyridin-3-ylmethyl-amine

Prepared from (4-chloro-6-morpholin-4-yl-pyrimidin-2-ylmethyl)-methyl-pyridin-3-ylmethyl-amine using Method D of Reference Example 2. Title compound obtained as an off-white solid (57 mg).

δ_{H} (400 MHz, CDCl₃) 2.48 (s, 3H), 3.75 (t, J=4.8, 4H), 3.84-3.88 (m, 8H), 6.86 (s, 1H), 7.02-7.03 (m, 1H), 7.17-7.20 (m, 1H), 7.25-7.30 (m, 2H), 7.38-7.42 (m, 1H), 7.84 (d, J=7.6, 1H), 8.31 (br s, 1H), 8.52-8.54 (m, 1H), 8.65 (s, 1H).

[M+H]⁺ 433.

### Example 46

### [4-(6-Methanesulfonyl-1H-indol-4-yl)-6-morpholin-4-yl-pyrimidin-2-ylmethyl]-methyl-pyridin-3-ylmethyl-amine

Prepared from (4-chloro-6-morpholin-4-yl-pyrimidin-2-ylmethyl)-methyl-pyridin-3-ylmethyl-amine using Method D of Reference Example 2. Title compound obtained as an off-white solid (21 mg).

δ_{H} (400 MHz, CDCl₃) 2.48 (s, 3H), 3.14 (s, 3H), 3.76-3.78 (m, 4H), 3.85-3.87 (m, 8H), 6.87 (s, 1H), 7.21 (s, 1H), 7.26-7.28 (m, 1H), 7.54-7.56 (m, 1H), 7.84 (d, J=7.6, 1H), 8.07 (s, 1H), 8.13 (s, 1H), 8.53 (d, J=4.8, 1H), 8.66 (s, 1H), 8.78 (br s, 1H).

[M+H]⁺ 493.

### Example 47

### 4-{2-[(Methyl-pyridin-3-ylmethyl-amino)-methyl]-6-morpholin-4-yl-pyrimidin-4-yl}-1H-indole-6-sulfonic acid dimethylamide

Prepared from (4-chloro-6-morpholin-4-yl-pyrimidin-2-ylmethyl)-methyl-pyridin-3-ylmethyl-amine using Method D. Title compound obtained as an off-white solid (70 mg).

δ_{H} (400 MHz, CDCl₃) 2.48 (s, 3H), 2.73 (s, 6H), 3.76-3.78 (m, 4H), 3.85-3.87 (m, 8H), 6.84 (s, 1H), 7.20 (s, 1H), 7.51-7.52 (m, 1H), 7.83 (d, J=7.6, 1H), 7.88 (s, 1H), 7.98 (s, 1H), 8.53 (d, J=4.8, 1H), 8.66 (s, 1H), 8.79 (br s, 1H).

[M+H]⁺ 522.

### Example 48

### 4-{2-[(Methyl-pyridin-3-ylmethyl-amino)-methyl]-6-morpholin-4-yl-pyrimidin-4-yl}-1H-indole-6-carboxylic acid amide

Prepared from (4-chloro-6-morpholin-4-yl-pyrimidin-2-ylmethyl)-methyl-pyridin-3-ylmethyl-amine using Method D of Reference Example 2. Title compound obtained as a pale brown solid (22 mg).

δ_{H} (400 MHz, d₆-DMSO, 92° C.) 2.42 (s, 3H), 3.73-3.76 (m, 10H), 3.83 (s, 2H), 7.10-7.12 (m, 2H), 7.29-7.33 (m, 1H), 7.47 (s, 1H), 7.80 (d, J=8.0, 1H), 8.09 (d, J=4.8, 1H), 8.44 (d, J=4.8, 1H), 8.58 (s, 1H), 10.29 (br s, 1H).

[M+H]⁺ 458.

### Example 49

### [4-(5-Fluoro-1H-indol-4-yl)-6-morpholin-4-yl-pyrimidin-2-ylmethyl]-methyl-pyridin-3-ylmethyl-amine

Prepared from (4-chloro-6-morpholin-4-yl-pyrimidin-2-ylmethyl)-methyl-pyridin-3-ylmethyl-amine using Method D of Reference Example 2. Title compound obtained as an off-white solid (52 mg).

δ_{H} (400 MHz, CDCl₃) 2.47 (s, 3H), 3.73 (t, J=4.8, 4H), 3.83-3.88 (m, 8H), 6.85 (d, J=2.0, 1H), 6.94 (br s, 1H), 7.04 (dd, J=11.2 and 8.8, 1H), 7.24-7.31 (m, 2H), 7.38 (dd, J=8.8 and 3.6, 1H), 7.83 (br d, J=7.2, 1H), 8.29 (br s, 1H), 8.52-8.53 (m, 1H), 8.65 (s, 1H).

[M+H]⁺ 433.

### Example 50

### [4-(1H-Indol-4-yl)-6-morpholin-4-yl-pyrimidin-2-ylmethyl]-methyl-quinolin-2-ylmethyl-amine

The title compound was prepared using the Suzuki conditions described in Method C of Reference Example 2 to give a yellow solid (38 mg).

δ_{H} (400 MHz, CDCl₃) 2.59 (s, 3H), 3.73 (t, 4H), 3.83 (t, 4H), 4.01 (s, 2H), 4.17 (s, 2H), 6.88 (s, 1H), 7.08 (s, 1H), 7.28-7.33 (m, 2H), 7.52 (m, 2H), 7.61 (d, 1H), 7.70 (t, 1H), 7.81 (d, 1H), 7.91 (d, 1H), 8.11 (m, 2H), 8.31 (br s, 1H).

[M+H]⁺ 465.18

### Example 51

### 1-[2-(1H-indole-4-yl)-6-morpholin-4-yl-pyrimidine-4-yl]-3-pyridin-3-yl-pyrrolidine

Prepared as a white solid from 3-pyrrolidine-3-yl-pyridine using Method G of Reference Example 2 (34 mg).

δ_{H} (400 MHz, CDCl₃) 2.15 (m, 1H); 2.45 (m, 1H); 3.53 (m, 1H); 3.69 (m, 4H); 3.81 (m, 2H); 3.83 (m, 4H); 4.03 (m, 1H); 4.26 (m, 1H); 6.42 (s, 1H); 7.19 (s, 1H); 7.28 (m, 2H); 7.46 (d, 1H); 7.64 (m, 2H); 8.28 (bs, 1H); 8.52 (d, 1H); 8.63 (s, 1H).

[M+H]⁺ 427.3

### Example 52

### Biological Testing

Compounds of the invention, prepared as described in the preceding Examples, were submitted to the following series of biological assays:

### (i) PI3K Biochemical Screening

Compound inhibition of PI3K was determined in a radiometric assay using purified, recombinant enzyme and ATP at a concentration of 1 uM. All compounds were serially diluted in 100% DMSO. The kinase reaction was incubated for 1 hour at room temperature, and the reaction was terminated by the addition of PBS. IC₅₀ values were subsequently determined using sigmoidal dose-response curve fit (variable slope). All of the compounds tested had an IC₅₀ against PI3K of 50 μM or less. Typically the IC₅₀ against PI3K was 5-500 nM.

### (ii) Cellular Proliferation Inhibition

Cells were seeded at optimal density in a 96 well plate and incubated for 4 days in the presence of test compound. Alamar Blue™ was subsequently added to the assay medium, and cells were incubated for 6 hours before reading at 544 nm excitation, 590 nm emission. EC₅₀ values were calculated using a sigmoidal dose response curve fit. All the compounds tested had an EC₅₀s of 50 uM or less in the range of cell lines utilized.

### Example 53

### Tablet Composition

Tablets, each weighing 0.15 g and containing 25 mg of a compound of the invention were manufactured as follows:

Composition for 10,000 tablets

Compound of the invention (250 g)

Lactose (800 g)

Corn starch (415 g)

Talc powder (30 g)

Magnesium stearate (5 g)

The compound of the invention, lactose and half of the corn starch were mixed. The mixture was then forced through a sieve 0.5 mm mesh size. Corn starch (10 g) is suspended in warm water (90 ml). The resulting paste was used to granulate the powder. The granulate was dried and broken up into small fragments on a sieve of 1.4 mm mesh size. The remaining quantity of starch, talc and magnesium was added, carefully mixed and processed into tablets.

### Example 54

### Injectable Formulation

| | |
|---|---|
| Compound of the invention | 200 mg |
| Hydrochloric Acid Solution 0.1M or | 4.0 to 7.0 |
| Sodium Hydroxide Solution 0.1M q.s. to pH | |
| Sterile water q.s. to | 10 ml |

The compound of the invention was dissolved in most of the water (35°-40° C.) and the pH adjusted to between 4.0 and 7.0 with the hydrochloric acid or the sodium hydroxide as appropriate. The batch was then made up to volume with water and filtered through a sterile micropore filter into a sterile 10 ml amber glass vial (type 1) and sealed with sterile closures and overseals.

### Example 55

### Intramuscular Injection

| | | |
|---|---|---|
| Compound of the invention | 200 | mg |
| Benzyl Alcohol | 0.10 | g |
| Glycofurol 75 | 1.45 | g |
| Water for injection q.s to | 3.00 | ml |

The compound of the invention was dissolved in the glycofurol. The benzyl alcohol was then added and dissolved, and water added to 3 ml. The mixture was then filtered through a sterile micropore filter and sealed in sterile 3 ml glass vials (type 1).

### Example 56

### Syrup Formulation

| | | |
|---|---|---|
| Compound of invention | 250 | mg |
| Sorbitol Solution | 1.50 | g |
| Glycerol | 2.00 | g |
| Sodium benzoate | 0.005 | g |
| Flavour | 0.0125 | ml |
| Purified Water q.s. to | 5.00 | ml |

The compound of the invention was dissolved in a mixture of the glycerol and most of the purified water. An aqueous solution of the sodium benzoate was then added to the solution, followed by addition of the sorbital solution and finally the flavour. The volume was made up with purified water and mixed well.

1. A compound which is a pyrimidine of formula (I): 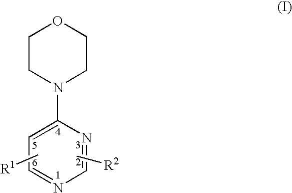
wherein
R² is bonded at ring position 2 and R¹ is bonded at ring position 5 or 6, or R¹ is bonded at ring position 2 and R² is bonded at ring position 6;
R¹ is selected from —(CR₂)ₘ—Y—R³, -[arylene-(CR₂)ₙ]ₚNR⁴R⁵, -[heteroarylene-(CR₂)ₙ]ₚ—NR⁴R⁵, —C(O)NR¹⁰R¹¹ and —O—(CR′R″)ₙ—R³;
R² is an indole group which is unsubstituted or substituted;
Y is selected from a direct bond, —O—(CR₂)ₙ—, —O—(CR₂)ₙ—NR—, —NR—(CR₂)ₙ—, NR—(CR₂)ₙO—(CR₂)ₙ—, —NR—(CR₂)ₙ—C(O)—, —(CR₂)—(CR₂)ₙ—, —S(O)_{q}(CR₂)ₙ—, —N(SO₂R)—(CR₂)ₙ—, NRC(O)—(CR₂)ₙ, —C(O)NR—(CR₂)ₙ—, —NRSO₂—(CR₂)ₙ, and —SO₂NR—(CR₂)ₙ;
m is 1, 2 or 3;
n is 0, 1, 2 or 3;
p is 0 or 1;
q is 0, 1 or 2;
each R, which are the same or different when more than one is present in a given group, is independently H or C₁-C₆ alkyl which is unsubstituted or substituted;
one of R′ and R″ is H and the other is C₁-C₆ alkyl which is unsubstituted or substituted, or each of R′ and R″, which are the same or different, is C₁-C₆ alkyl which is unsubstituted or substituted;
R³ is selected from an unsaturated 5- to 12-membered carbocyclic or heterocyclic ring, a saturated 5-, 6- or 7-membered N-containing heterocyclic group which is unsubstituted or substituted, a group —OR and a group —NR⁶R⁷;
one of R⁴ and R⁵ is H and the other is a saturated 5-, 6- or 7-membered N-containing heterocyclic group which is unsubstituted or substituted, or one of R⁴ and R⁵ is unsubstituted C₁-C₆ alkyl and the other is C₁-C₆ alkyl substituted by an unsaturated 5- to 12-membered carbocyclic or heterocyclic ring which is unsubstituted or substituted, or R⁴ and R⁵, which are the same or different, are both C₁-C₆ alkyl substituted by an unsaturated 5- to 12-membered carbocyclic or heterocyclic ring which is unsubstituted or substituted, or R⁴ and R⁵ together form, with the nitrogen atom to which they are attached, a saturated 5-, 6- or 7-membered N-containing heterocyclic group which is unsubstituted or substituted or which is fused to a benzene ring;
R⁶ and R⁷, which are the same or different, are each independently selected from H and
C₁-C₆ alkyl which is unsubstituted or substituted, or R⁶ and R⁷ together form, with the nitrogen atom to which they are attached, a saturated 5-, 6- or 7-membered N-containing heterocyclic ring which is unsubstituted or substituted or which is fused to a second saturated 5-, 6- or 7-membered N-containing heterocyclic ring; and
R¹⁰ and R¹¹, which are the same or different, are each C₁-C₆ alkyl which is unsubstituted or substituted, or one of R¹⁰ and R¹¹ is H and the other is a saturated 5-, 6- or 7-membered N-containing heterocyclic group which is unsubstituted or substituted, or one of R¹⁰ and R¹¹ is unsubstituted C₁-C₆ alkyl and the other is C₁-C₆ alkyl substituted by an unsaturated 5- to 12-membered carbocyclic or heterocyclic ring which is unsubstituted or substituted, or R¹⁰ and R¹¹, which are the same or different, are both C₁-C₆ alkyl substituted by an unsaturated 5- to 12-membered carbocyclic or heterocyclic ring which is unsubstituted or substituted, or R¹⁰ and R¹¹ together form, with the nitrogen atom to which they are attached, a saturated 5-, 6- or 7-membered N-containing heterocyclic group which is unsubstituted or substituted or which is fused to a benzene ring;
or a pharmaceutically acceptable salt thereof;
with the proviso that when one of R⁴ and R⁵ is unsubstituted C₁-C₆ alkyl and the other is C₁-C₆ alkyl substituted by an unsaturated 5- to 12-membered carbocyclic or heterocyclic ring which is unsubstituted or substituted, or R⁴ and R⁵, which are the same or different, are both C₁-C₆ alkyl substituted by an unsaturated 5- to 12-membered carbocyclic or heterocyclic ring which is unsubstituted or substituted, then R² is other than an indol-4-yl group which is substituted at the 5- or 6-position.

2. A compound according to claim 1 wherein the pyrimidine is of formula (Ia): 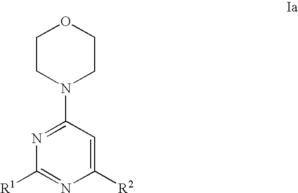
wherein R¹ and R² are as defined in claim 1.

3. A compound according to claim 1 wherein the pyrimidine is of formula (Ib): 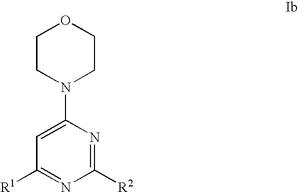
wherein R¹ and R² are as defined in claim 1.

4. A compound according to claim 1 wherein the pyrimidine is of formula (Ic): 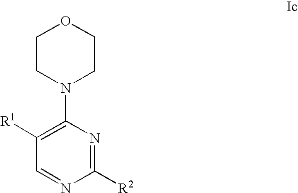
wherein R¹ and R² are as defined in claim 1.

5. A compound according to claim 1 wherein R¹ is selected from —(CR₂)ₘ—Y—R³ and —C(O)NR¹⁰R¹¹ wherein R, m, R³, R¹⁰ and R¹¹ are as defined in claim 1.

6. A compound according to claim 1 wherein R² is an indole group which is unsubstituted or substituted by a group selected from CN, halo, —C(O)NR₂, halo(C₁-C₆)alkyl, —SO₂R, —SO₂NR₂, and a 5-membered heteroaryl group containing 1, 2, 3 or 4 heteroatoms selected from O, N and S, wherein R is H or C₁-C₆ alkyl.

7. A compound which is selected from:
N-[4-(6-Fluoro-1H-indol-4-yl)-6-morpholin-4-yl-pyrimidin-2-ylmethyl]-nicotinamide;
4-(6-Fluoro-1H-indol-4-yl)-6-morpholin-4-yl-pyrimidin-2-ylmethyl]-pyridin-3-ylmethyl-amine;
Piperidine-4-carboxylic acid [4-(6-fluoro-1H-indol-4-yl)-6-morpholin-4-yl-pyrimidin-2-ylmethyl]-amide;
4-[2-(Hexahydro-pyrrolo[3,4-c]pyrrol-2-ylmethyl)-6-morpholin-4-yl-pyrimidin-4-yl]-1H-indole;
4-(1H-Indol-4-yl)-6-morpholin-4-yl-pyrimidine-2-carboxylic acid dimethylamide;
4-[6-Morpholin-4-yl-2-(pyridin-3-ylmethoxymethyl)-pyrimidin-4-yl]-1H-indole;
{2-[4-(1H-Indol-4-yl)-6-morpholin-4-yl-pyrimidin-2-yl]-ethyl}-(5-trifluoromethyl-pyridin-2-yl)-amine;
N-[4-(1H-Indol-4-yl)-6-morpholin-4-yl-pyrimidin-2-ylmethyl]-ylmethyl-methanesulfonamide;
Pyridine-3-sulfonic acid [4-(1H-indol-4-yl)-6-morpholin-4-yl-pyrimidin-2-ylmethyl]-methyl-amide;
[4-(6-Fluoro-1H-indol-4-yl)-6-morpholin-4-yl-pyrimidin-2-ylmethyl]-phenethyl-amine;
N′-[4-(6-Fluoro-1H-indol-4-yl)-6-morpholin-4-yl-pyrimidin-2-ylmethyl]-N,N-dimethyl-ethane-1,2-diamine;
[4-(6-Fluoro-1H-indol-4-yl)-6-morpholin-4-yl-pyrimidin-2-ylmethyl]-(2-methoxy-ethyl)-amine;
[4-(6-Fluoro-1H-indol-4-yl)-6-morpholin-4-yl-pyrimidin-2-ylmethyl]-[2-(3H-imidazol-4-yl)-ethyl]-amine;
Benzyl-[4-(6-fluoro-1H-indol-4-yl)-6-morpholin-4-yl-pyrimidin-2-ylmethyl]-amine;
[4-(6-Fluoro-1H-indol-4-yl)-6-morpholin-4-yl-pyrimidin-2-yl]-piperazin-1-yl-methanone;
4-(6-Fluoro-1H-indol-4-yl)-6-morpholin-4-yl-pyrimidine-2-carboxylic acid piperidin-4-ylamide;
6-Fluoro-4-[6-morpholin-4-yl-2-(5-piperazin-1-ylmethyl-thiophen-3-yl)-pyrimidin-4-yl]-1H-indole;
6-Fluoro-4-[6-morpholin-4-yl-2-(3-piperazin-1-yl-phenyl)-pyrimidin-4-yl]-1H-indole;
2-[4-(1H-Indol-4-yl)-6-morpholin-4-yl-pyrimidin-2-ylmethyl]-1,2,3,4-tetrahydro-isoquinoline;
1-[4-(1H-Indol-4-yl)-6-morpholin-4-yl-pyrimidin-2-ylmethyl]-1,2,3,4,5,6-hexahydro-[4,4]bipyridinyl;
4-[6-Morpholin-4-yl-2-(2-pyridin-3-yl-ethyl)-pyrimidin-4-yl]-1H-indole;
4-[4-(4-Methyl-piperazin-1-yl)-6-morpholin-4-yl-pyrimidin-2-yl]-1H-indole;
[2-(1H-Indol-4-yl)-6-morpholin-4-yl-pyrimidin-4-yl]-methyl-phenethyl-amine;
2-(1H-Indol-4-yl)-6-morpholin-4-yl-pyrimidin-4-ylmethyl]-phenethyl-amine;
[2-(1H-Indol-4-yl)-6-morpholin-4-yl-pyrimidin-4-ylmethyl]-dimethyl-amine;
Benzyl-[2-(1H-indol-4-yl)-6-morpholin-4-yl-pyrimidin-4-ylmethyl]-methyl-amine;
Benzyl-[2-(1H-indol-4-yl)-6-morpholin-4-yl-pyrimidin-4-ylmethyl]-amine;
[2-(1H-Indol-4-yl)-6-morpholin-4-yl-pyrimidin-4-ylmethyl]-methyl-pyridin-3-ylmethyl-amine;
[2-(1H-Indol-4-yl)-6-morpholin-4-yl-pyrimidin-4-ylmethyl]-pyridin-3-ylmethyl-amine;
[2-(1H-Indol-4-yl)-6-morpholin-4-yl-pyrimidin-4-ylmethyl]-(2-methoxy-ethyl)-amine;
[2-(1H-imidazol-4-yl)-ethyl]-[2-(1H-indol-4-yl)-6-morpholin-4-yl-pyrimidin-4-ylmethyl]amine;
[2-(1H-Indol-4-yl)-6-morpholin-4-yl-pyrimidin-4-ylmethyl]-(1-phenyl-ethyl)-amine;
[2-(1H-Indol-4-yl)-6-morpholin-4-yl-pyrimidin-4-ylmethyl]-(2-morpholin-4-yl-ethyl)-amine;
[2-(6-Fluoro-1H-indol-4-yl)-6-morpholin-4-yl-pyrimidin-4-ylmethyl]methyl-pyridin-3-ylmethyl-amine;
[2-(6-Methanesulfonyl-1H-indol-4-yl)-6-morpholin-4-yl-pyrimidin-4-ylmethyl]methyl-pyridin-3-ylmethyl-amine;
[2-(5-Fluoro-1H-indol-4-yl)-6-morpholin-4-yl-pyrimidin-4-ylmethyl]methyl-pyridin-3-ylmethyl-amine;
[4-(1H-Indol-4-yl)-6-morpholin-4-yl-pyrimidin-2-ylmethyl]-methyl-thiophen-2-ylmethyl-amine;
(1-Benzyl-piperidin-4-yl)-[4-(1H-indol-4-yl)-6-morpholin-4-yl-pyrimidin-2-yl]-amine;
4-[4-(4-Methyl-piperazin-1-yl)-6-morpholin-4-yl-pyrimidin-2-yl]-1H-indole;
1-[2-(1H-Indol-4-yl)-6-morpholin-4-yl-pyrimidin-4-yl]-4-phenyl-piperidin-4-ol;
1-[2-(1H-Indol-4-yl)-6-morpholin-4-yl-pyrimidin-4-yl]-piperidine-4-carboxylic acid ethyl ester;
1-[2-(1H-Indol-4-yl)-6-morpholin-4-yl-pyrimidin-4-yl]-4-phenyl-piperidine-4-carbonitrile;
Methyl-[4-morpholin-4-yl-6-(6-trifluoromethyl-1H-indol-4-yl)-pyrimidin-2-ylmethyl]-pyridin-3-ylmethyl-amine;
[4-(6-Fluoro-1H-indol-4-yl)-6-morpholin-4-yl-pyrimidin-2-ylmethyl]-methyl-pyridin-3-ylmethyl-amine;
[4-(6-Methanesulfonyl-1H-indol-4-yl)-6-morpholin-4-yl-pyrimidin-2-ylmethyl]-methyl-pyridin-3-ylmethyl-amine;
4-{2-[(Methyl-pyridin-3-ylmethyl-amino)-methyl]-6-morpholin-4-yl-pyrimidin-4-yl-1H-indole-6-sulfonic acid dimethylamide;
4-{2-[(Methyl-pyridin-3-ylmethyl-amino)-methyl]-6-morpholin-4-yl-pyrimidin-4-yl]-1H-indole-6-carboxylic acid amide;
[4-(5-Fluoro-1H-indol-4-yl)-6-morpholin-4-yl-pyrimidin-2-ylmethyl]-methyl-pyridin-3-ylmethyl-amine;
[4-(1H-Indol-4-yl)-6-morpholin-4-yl-pyrimidin-2-ylmethyl]-methyl-quinolin-2-ylmethyl-amine;
1-[2-(1H-indole-4-yl)-6-morpholin-4-yl-pyrimidine-4-yl]-3-pyridin-3-yl-pyrrolidine;
and the pharmaceutically acceptable salts thereof.

8. A pharmaceutical composition which comprises a pharmaceutically acceptable carrier or diluent and, as an active ingredient, a compound as defined in claim 1.

9-12. (canceled)

13. A method of treating a disease or disorder arising from abnormal cell growth, function or behaviour associated with P13 kinase, which method comprises administering to a patient in need thereof a compound as defined in claim 1.

14. A method according to claim 13 wherein the disease or disorder is selected from cancer, immune disorders, cardiovascular disease, viral infection, inflammation, metabolism/endocrine function disorders and neurological disorders.
